(19) United States
(12) Patent Application Publication
(10) Pub. No.: US 2011/0245164 A1
(43) Pub. Date: Oct. 6, 2011

(54) YL-BASED INSULIN-LIKE GROWTH FACTORS EXHIBITING HIGH ACTIVITY AT THE INSULIN RECEPTOR

(75) Inventors: Richard D. DiMarchi, Carmel, IN (US); Shujiang Cheng, Bloomington, IN (US); Binbin Kou, Bloomington, IN (US); Jie Han, Bloomington, IN (US)

(73) Assignee: INDIANA UNIVERSITY RESEARCH AND TECHNOLOGY CORPORATION, INDIANAPOLIS, IN (US)

(21) Appl. No.: 13/130,960

(22) PCT Filed: Dec. 18, 2009
(86) PCT No.: PCT/US09/68713

§ 371 Date: May 24, 2011

Related U.S. Application Data
(60) Provisional application No. 61/139,223, filed on Dec. 19, 2008.

(51) Int. Cl. A61K 38/28; C07K 14/62; A61K 38/30; A61P 3/10; C07K 14/65

(52) U.S. Cl. 514/6.1; 530/303; 514/8.5; 514/6.9; 530/324

(57) ABSTRACT

Insulin-like growth factor analogs are disclosed wherein substitution of the IGF native amino acids, at positions corresponding to positions B16 and B17 of native insulin, with tyrosine and leucine, respectively, increases potency of the resulting analog at the insulin receptor by tenfold. Also disclosed are prodrug and depot formulations of the IGF analogs, wherein the IGF analog has been modified by the linkage of a dipeptide to the analog through an amide bond linkage. The prodrug and depot formulations disclosed herein have extended half lives of at least 2 hours, 10 hours, and more typically greater than 2 are converted to the active form at physiological conditions through a non-enzymatic reaction driven by chemical instability.

### CROSS REFERENCE TO RELATED APPLICATIONS

This application claims priority to U.S. Provisional Patent Application No. 61/139,223 filed on Dec. 19, 2008, the disclosure of which is hereby expressly incorporated by reference in its entirety.

### BACKGROUND

Insulin is a proven therapy for the treatment of juvenile-onset diabetes and later stage adult-onset diabetes. Unfortunately, its pharmacology is not glucose sensitive and as such it is capable of excessive action that can lead to life-threatening hypoglycemia. Inconsistent pharmacology is a hallmark of insulin therapy such that it is extremely difficult to normalize blood glucose without occurrence of hypoglycemia. Furthermore, native insulin is of short duration of action and requires modification to render it suitable for use in control of basal glucose. One central goal in insulin therapy is designing an insulin formulation capable of providing a once a day time action. Extending the action time of an insulin dosage can be achieved by decreasing the solubility of insulin at the site of injection.

There are three proven and distinct molecular approaches to reducing solubility and they include; (1) formulation of insulin as an insoluble suspension with zinc, (2) increase in its isoelectric point to physiological pH through addition of cationic amino acids, (3) covalent modification to provide a hydrophobic ligand that reduces solubility and binds albumin. All of these approaches are limited by the inherent variability that occurs with precipitation at the site of injection, and with subsequent re-solubilization & transport to blood as an active hormone.

Prodrug chemistry offers an alternative mechanism to precisely control the onset and duration of insulin action after clearance from the site of administration and equilibration in the plasma at a highly defined concentration. The central virtue of such an approach relative to current long-acting insulin analogs and formulations is that the insulin reservoir is not the subcutaneous fatty tissue where injection occurs, but rather the blood compartment. This removes the variability in precipitation and solubilization. The use of a prodrug form of insulin also enables administration of the peptide hormone by routes other than a subcutaneous injection. To build a successful prodrug-hormone, an active site structural address is needed that can form the basis for the reversible attachment of a prodrug structural element. The structural address needs to offer two key features; (1) the potential for selective chemical modification and (2) the ability to provide full activity in the native form upon removal of the prodrug structural element.

Insulin is a two chain heterodimer that is biosynthetically derived from a low potency single chain proinsulin precursor through enzymatic processing. Human insulin is comprised of two peptide chains (an “A chain” (SEQ ID NO: 1) and “B chain” (SEQ ID NO: 2)) bound together by disulfide bonds and having a total of 51 amino acids. The native insulin structure has limited unique chemical elements at the active site residues that might be used for selective assemble of an amide linked prodrug element. Accordingly there is a need for insulin mimetics that function as insulin receptor agonists but have advantageous properties such as providing sites for attachment of prodrug elements, enhanced ease of synthesis, and co-agonist activity at receptors other than the insulin receptors.

Insulin-like growth factors (IGF's) have been isolated from various animal species and are believed to be active growth promoting molecules that mediate the anabolic effects of such hormones as growth hormone and placental lactogen. To date, several classes of IGF's have been identified. These include insulin-like growth factor-I (IGF-1; somatomedin C), insulin-like growth factor-II (IGF-2; Somatomedin A) and a mixture of peptides called “multiplication-stimulating activity.” This heterologous group of peptides exhibit important growth-promoting effects in vitro (Daughaday, W. H. (1977) Clin. Endocrin. Metab. 6: 117-135.; Clemmons, D. R. and Van Wyk, J. J. (1981) J. Cell Physiol. 106: 362-367.) and in vivo (Schoenle, E. Zapf, J., Humbel, R. E. and Froesch, E. R. (1982) Nature 296: 252-253).

Human IGF-1 is a 70 aa basic peptide having the protein sequence shown in SEQ ID NO: 3, and has a 43% homology with proinsulin (Rinderknecht et al. (1978) J. Biol. Chem. 253:2769-2776). Human IGF-2 is a 67 amino acid basic peptide having the protein sequence shown in SEQ ID NO: 4. Specific binding proteins of high molecular weight having very high binding capacity for IGF-1 and IGF-2 act as carrier proteins or as modulators of IGF-1 functions (Holly et al. (1989) J. Endocrinol. 122:611-618).

Applicants have identified YL based IGF analogs (referred to herein as IGF^{B16B17} derivative peptides) that display high activity at the insulin receptor. Such derivatives are more readily synthesized than insulin and enable the development of co-agonist analogs for insulin and IGF-1 receptors, and potentially selective insulin receptor isoform specific analogs.

### SUMMARY

As disclosed herein the B16 tyrosine of insulin has been identified as an amino acid of great importance to high affinity insulin agonism. Selective substitution of the native IGF residues corresponding to positions B16 and B17 of native insulin with the tyrosine and leucine, respectively, increases potency of the resulting IGF analog at the insulin receptor by tenfold. Accordingly, the remaining differences in amino acid sequence between insulin and IGFs appear to be of minor importance to high affinity interaction of insulin-like ligands with the insulin receptor. This discovery enables the use of IGF-insulin based hybridized peptides to be used as full and super-potent insulin agonists. The newly discovered importance of B16 tyrosine in these peptides identify it as a site for selective assemble of insulin-agonist prodrugs. Additional virtues of the IGF^{B16B17} derivative peptide include, but are not limited to relative ease of synthesis, development of co-agonists for insulin and IGF-1 receptors, and potentially selective insulin receptor isoform specific analogs.

In accordance with one embodiment an analog of IGF proteins exhibiting full potency at the insulin receptor is provided wherein the IGF analog has the dipeptide Tyr-Leu substituting for the native amino acids of IGF-1 and IGF-2 at positions corresponding to B16 and B17 of native insulin. In accordance with one embodiment an IGF analog is provided comprising the sequence

| | |
|---|---|
| X₂₅LCGX₂₉X₃₀LVX₃₃X₃₄LYLVCGDX₄₂GFY | (SEQ ID NO: 9) |

wherein X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine; and

X₄₂ is selected from the group consisting of alanine, ornithine and arginine. In one embodiment the IGF analog further comprises a second peptide linked to the peptide of SEQ ID NO: 9 either by intramolecular disulfide bonds or the two peptides are covalently linked to one another through a peptide bond to form a contiguous single chain amino acid sequence. In one embodiment the second peptide comprises the sequence GIVX₄ECCX₈X₉SCDLX₁₄X₁₅LEX₁₈X₁₉CX₂₁—R₁₃ (SEQ ID NO: 19) wherein

X₄ is glutamic acid or aspartic acid;

X₈ is histidine or phenylalanine;

X₉ and X₁₄ are independently selected from ornathine, arginine or alanine;

X₁₅ is arginine, alanine, ornathine or leucine;

X₁₈ is methionine, asparagine or threonine;

X₁₉ is tyrosine, or 4-amino phenylalanine;

X₂₁ is alanine, glycine or asparagine; and

R₁₃ is COOH or CONH₂.

In accordance with one embodiment an IGF^{B16B17} derivative peptide is provided comprising an A chain having the sequence GIVX₄X₅CCX₈X₉X₁₀CX₁₂LX₁₄X₁₅LEX₁₈X₁₉CX₂₁—R₁₃ (SEQ ID NO: 82) and a B chain having the sequence X₂₅LCGX₂₉X₃₀LVX₃₃X₃₄LYLVCGX₄₁X₄₂GFX₄₅R₄₇—R₄₈—R₄₉—R₁₄ (SEQ ID NO: 67), wherein

X₄ is glutamic acid or aspartic acid;

X₅ is glutamic acid or glutamine;

X₈ is histidine, threonine or phenylalanine;

X₉ is serine, ornathine, arginine or alanine;

X₁₀ is serine or isoleucine;

X₁₂ is serine or aspartic acid;

X₁₄ are independently selected from tyrosine, ornathine, arginine or alanine;

X₁₅ is glutamine, ornathine, arginine, alanine or leucine;

X₁₈ is methionine, asparagine or threonine;

X₁₉ is tyrosine, 4-methoxy-phenylalanine or 4-amino phenylalanine;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid and aspartic acid;

X₄₂ is selected from the group consisting of alanine, ornithine and arginine;

X₄₅ is phenylalanine or tyrosine;

R₁₃ is COOH or CONH₂;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a proline-arginine dipeptide, a lysine-proline dipeptide, or a proline-lysine dipeptide;

R₄₉ is threonine or alanine; and R₁₃ and R₁₄ are independently selected from COOH and CONH₂, with the proviso that the B chain is not a native insulin B chain sequence (e.g., not SEQ ID NO: 2).

In accordance with one embodiment an IGF^{B16B17} derivative peptide is provided comprising an A chain having the sequence GIVDECCX₈X₉SCDLRRLEMX₁₉CX₂₁—R₁₃ (SEQ ID NO: 21) and a B chain having the sequence R₂₂—X₂₅LCGX₂₉X₃₀LVX₃₃X₃₄LX₃₆LVCGDX₄₂GFX₄₅—R₄₇—R₄₈—R₄₉—R₁₄ (SEQ ID NO: 20), wherein

X₈ is phenylalanine or histidine;

X₉ is arginine or alanine;

X₁₉ is tyrosine;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₃₆ is tyrosine;

X₄₂ is selected from the group consisting of alanine, ornithine and arginine;

X₄₅ is tyrosine;

R₂₂ is selected from the group consisting of the tripeptide glycine-proline-glutamic acid, the dipeptide proline-glutamic acid, glutamic acid and an N-terminal amine;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a lysine-proline dipeptide, or a proline-lysine dipeptide;

R₄₉ is threonine or alanine; and R₁₃ and R₁₄ are independently selected from COOH and CONH₂.

In accordance with one embodiment a prodrug derivative of an IGF^{B16B17} derivative peptide is provided. In one embodiment such peptide comprises a modified IGF A chain and B chain, wherein the A chain comprises a sequence of Z-GIVX₄ECCX₈X₉SCDLX₁₄X₁₅LEX₁₈X₁₉CX₂₁—R₁₃ (SEQ ID NO: 19) or a sequence that differs from SEQ ID NO: 19 by 1 to 3 amino acid modifications selected from positions 5, 8, 9, 10, 12, 14, 15, 17, 18 and 21 of SEQ ID NO: 19, and said B chain sequence comprises a sequence of J-R₂₂—X₂₅LCGX₂₉X₃₀LVX₃₃X₃₄LX₃₆LVCGDX₄₂GFX₄₅—R₁₄ (SEQ ID NO: 20) or a sequence that differs from SEQ ID NO: 20 by 1 to 3 amino acid modifications selected from positions 5, 6, 9, 10, 16, 17, 18, 19 and 21 of SEQ ID NO: 20; wherein Z and J are independently Hydrogen (forming an N-terminal amine)or a dipeptide comprising the general structure of Formula I:

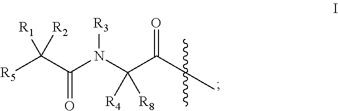

wherein

R_{1,} R_{2,} R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl, and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (Cₒ-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl) (C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH;

X₄ is aspartic acid or glutamic acid;

X₈ is histidine or phenylalanine;

X₉ and X₁₄ are independently selected from arginine or alanine;

X₁₅ is arginine or leucine;

X₁₈ is methionine, asparagine or threonine;

X₁₉ is an amino acid of the general structure

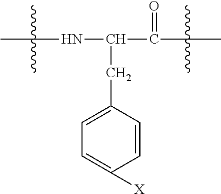

wherein X is selected from the group consisting of OH or NHR₁₀, wherein R₁₀ is a dipeptide comprising the general structure of Formula I:

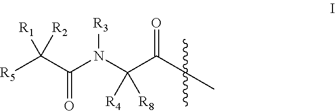

X₂₁ is alanine, glycine or asparagine;

R₂₂ is a covalent bond or 1 to six amino acids;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₃₆ is an amino acid of the general structure

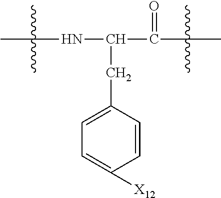

wherein X₁₂ is selected from the group consisting of OH and NHR₁₁, wherein R₁₁ is a dipeptide comprising the general structure of Formula I:

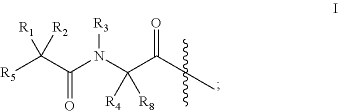

X₄₂ is selected from the group consisting of alanine and arginine.;

X₄₅ is an amino acid of the general structure

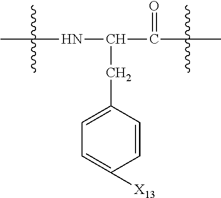

wherein X₁₃ is selected from the group consisting of OH and NHR₁₂, wherein R₁₂ is a dipeptide comprising the general structure of Formula I:

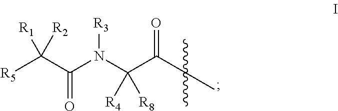

and

R₁₃ and R₁₄ are independently COOH or CONH₂, with the proviso that one and only one of X, X₁₂, X₁₃, J and Z comprises a dipeptide of the general structure of Formula I:

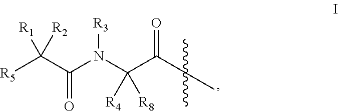

and that said IGF^{B16B17} derivative peptide does not comprise the sequence of SEQ ID NO: 1 or SEQ ID NO: 2. In one embodiment, when J or Z comprise the dipeptide of Formula I, and R₄ and R₃ together with the atoms to which they are attached form a 4, 5 or 6 member heterocyclic ring, then both R₁ and R₂ are not hydrogen. In accordance with one embodiment R₂₂ is selected from the group consisting of the peptide AYRPSE (SEQ ID NO: 14), FGPE (SEQ ID NO: 68), the tripeptide glycine-proline-glutamic acid, the dipeptide proline-glutamic acid, glutamic acid and an N-terminal amine. In accordance with one embodiment R₂₂ is selected from the group consisting of a tripeptide glycine-proline-glutamic acid, a dipeptide proline-glutamic acid, glutamic acid and an N-terminal amine.

In accordance with one embodiment the dipeptide present at Z, J, R₁₀, R₁₁ or R₁₂ comprises a compound having the general structure of Formula I:

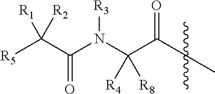

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo, with the proviso that when J or Z comprise the dipeptide of Formula I, and R₄ and R₃ together with the atoms to which they are attached form a 4, 5 or 6 member heterocyclic ring, then both R₁ and R₂ are not hydrogen.

In accordance with one embodiment, X₁₂ and X₁₃ are each OH and J and Z are each H and X comprises a dipeptide of the general structure of Formula I:

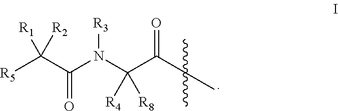

In one embodiment the IGF^{B16B17} derivative peptide comprises an A chain having the sequence of Z-GIVDECCFRSCDLRRLEMX₁₉CA-R₁₃ and a B chain having the sequence J-R₂₂-TLCGAELVDALX₃₆LVCGDRGFX₄₅FNKPX₄₉-R₁₄, wherein the designations are defined as above.

In accordance with one embodiment the dipeptide structure of Formula I further comprises a large molecule covalently bound to the dipeptide that prevents the IGF^{B16B17} derivative peptide from interacting with the insulin or IGF receptor upon administration to a patient. Subsequent cleavage of the dipeptide from the IGF^{B16B17} derivative peptide releases the peptide in a fully active form. In accordance with one embodiment the dipeptide structure of Formula I further comprises a polymer (e.g. a hydrophilic polymer), an alkyl or acylating group.

In accordance with one embodiment single-chain IGF^{B16B17} derivative peptides, and prodrug derivatives thereof, are provided. In this embodiment the carboxy terminus of an IGF analog B chain of the present disclosure, or a functional analog thereof, is covalently linked to the N-terminus of an IGF A chain, or a functional analog thereof. In one embodiment the B chain is linked to the A chain via peptide linker of 4-12 or 4-8 amino acids.

In another embodiment the solubility of the IGF^{B16B17} derivative peptides is enhanced by the covalent linkage of a hydrophilic moiety to the peptide. In one embodiment the hydrophilic moiety is linked to either the N-terminal amino acid of the B chain or to the amino acid at position 27 of SEQ ID NO: 6. In one embodiment the hydrophilic moiety is a polyethylene glycol (PEG) chain, having a molecular weight selected from the range of about 500 to about 40,000 Daltons. In one embodiment the polyethylene glycol chain has a molecular weight selected from the range of about 500 to about 5,000 Daltons. In another embodiment the polyethylene glycol chain has a molecular weight of about 10,000 to about 20,000 Daltons.

Acylation or alkylation can increase the half-life of the IGF^{B16B17} derivative peptides, and prodrug derivatives thereof, in circulation. Acylation or alkylation can advantageously delay the onset of action and/or extend the duration of action at the insulin receptors. The insulin analogs may be acylated or alkylated at the same amino acid position where a hydrophilic moiety is linked, or at a different amino acid position.

In accordance with one embodiment a pharmaceutical composition is provided comprising any of the novel IGF^{B16B17} derivative peptides disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain an IGF^{B16B17} derivative peptide as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

In accordance with one embodiment an improved method of regulating blood glucose levels in insulin dependent patients is provided. The method comprises the steps of administering an IGF^{B16B17} derivative peptide of the present disclosure, or prodrug derivative thereof, in an amount therapeutically effective for the control of diabetes. In one embodiment the IGF^{B16B17} derivative peptide is pegylated with a PEG chain having a molecular weight selected from the range of about 5,000 to about 40,000 Daltons

### BRIEF DESCRIPTION OF THE DRAWINGS

FIG. 1 is a schematic overview of the two step synthetic strategy for preparing human insulin. Details of the procedure are provided in Example 1.

FIG. 2 is a graph comparing insulin receptor specific binding of synthetic human insulin relative to purified native insulin. As indicated by the data presented in the graph, the two molecules have similar binding activities.

FIG. 3 is a graph comparing relative insulin receptor binding of native insulin and the A19 insulin analog (Insulin(p-NH₂—F)¹⁹). As indicated by the data presented in the graph, the two molecules have similar binding activities.

FIG. 4 is a graph comparing relative insulin receptor binding of native insulin and the IGF1(Y^{B16}L^{B17}) analog. As indicated by the data presented in the graph, the two molecules have similar binding activities.

FIG. 5 is an alignment of the human proinsulin (SEQ ID NO: 66) and insulin-like growth factors I and II (IGF I; SEQ ID NO: 3 and IGF II; SEQ ID NO: 4) amino acid sequences. The alignment demonstrates that these three peptides share a high level of sequence identity (* indicates a space with no corresponding amino acid and a dash (−) indicates the identical amino acid as present in insulin).

FIG. 6 is a schematic drawing of the synthetic scheme used to prepare the IGF1(Y^{B16B17})(p-NH₂—F)^{A19} prodrug analogs.

FIG. 7 is a graph comparing relative insulin receptor binding of IGF1(Y^{B16}L¹⁷)(p-NH₂—F)^{A19} and the dipeptide extended form of IGF1(Y^{B16}L^{B17})(p-NH₂—F)^{A19}-AiBAla, wherein the dipeptide AiBAla is bound at position A19 (i.e. IGF1(Y^{B16}L^{B17})(AiBAla).

FIG. 8A-8C provides the activity of a dimer prepared in accordance with the present disclosure. FIG. 8A shows the structure of an IGF-1 single chain dimer that comprises two single chain IGF^{B16B17} derivative peptides (IGF-1B chain[C⁰H⁵Y¹⁶L¹⁷O²²]-A chain[O^{9,14,15}N^{18,21}]; SEQ ID NO: 83) linked together by a disulfide bond between the side chains of the amino terminus of the B chains. FIG. 8B is a graph demonstrating the relative insulin receptor binding of insulin, IGF-1, a single chain IGF^{B16B17} derivative peptide dimer and a two chain IGF^{B16B17} derivative peptide dimer. FIG. 8C is a graph demonstrating the relative activity of insulin, IGF-1, and a two chain IGF^{B16B17} derivative peptide dimer to induce insulin receptor phosphorylation.

FIG. 9A-9C shows the degradation of a prodrug form of an IGF^{B16B17} derivative peptide: (Aib-Pro on (pNH₂—F)¹⁹ of IGF1A(Ala)^{6,7,11,20}amide. The dipeptide was incubated in PBS, pH 7.4 at 37° C. for predetermined lengths of time. Aliquots were taken at 20 minutes (FIG. 9A), 81 minutes (FIG. 9B) and 120 minutes (FIG. 9C) after beginning the incubation, were quenched with 0.1% TFA and tested by analytical HPLC. Peak a (IGF1A(Ala)^{6,7,11,20}(pNH₂—F)¹amide) and b (IGF1A(Ala)^{6,7,11,20}(Aib-Pro-pNH—F)¹⁹amide) were identified with LC-MS and quantified by integration of peak area. The data indicate the spontaneous, non-enzymatic conversion of IGF1A(Ala)^{6,7,11,20}(Aib-Pro-pNH—F)¹⁹amide to IGF1A(Ala)6,7,11,20(pNH₂—F)¹amide over time.

FIGS. 10A & 10B are graphs depicting the in vitro activity of the prodrug Aib,dPro-IGF1YL (dipeptide linked throught the A19 4-aminoPhe). FIG. 10A is a graph comparing relative insulin receptor binding of native insulin (measured at 1 hour at 4° C.) and the A19 IGF prodrug analog (Aib,dPro-IGF1YL) over time (0 hours, 2.5 hours and 10.6 hours) incubated in PBS. FIG. 10B is a graph comparing relative insulin receptor binding of native insulin (measured at 1.5 hour at 4° C.) and the A19 IGF prodrug analog (Aib,dPro-IGF1YL) over time (0 hours, 1.5 hours and 24.8 hours) incubated in 20% plasma/PBS. As indicated by the data presented in the graph, increased activity is recovered form the A19 IGF prodrug analog sample as the prodrug form is converted to the active IGF1YL peptide.

FIGS. 11A & 11B are graphs depicting the in vitro activity of the prodrug dK,(N-isobutylG)-IGF1YL (dipeptide linked throught the A19 4-aminoPhe). FIG. 11A is a graph comparing relative insulin receptor binding of native insulin (measured at 1 hour at 4° C.) and the A19 IGF prodrug analog (IGF1YL: dK,(N-isobutylG) over time (0 hours, 5 hours and 52 hours) incubated in PBS. FIG. 11B is a graph comparing relative insulin receptor binding of native insulin (measured at 1.5 hour at 4° C.) and the A19 IGF prodrug analog (IGF1YL: dK,(N-isobutylG) over time (0 hours, 3.6 hours and 24.8 hours) incubated in 20% plasma/PBS. As indicated by the data presented in the graph, increased activity is recovered form the A19 IGF prodrug analog sample as the prodrug form is converted to the active IGF1YL peptide.

FIGS. 12A & 12B are graphs depicting the in vitro activity of the prodrug dK(e-acetyl),Sar)-IGF1YL (dipeptide linked throught the A19 4-aminoPhe). FIG. 12A is a graph comparing relative insulin receptor binding of native insulin (measured at 1 hour at 4° C.) and the A19 IGF prodrug analog (IGF1YL: dK(e-acetyl),Sar) over time (0 hours, 7.2 hours and 91.6 hours) incubated in PBS. FIG. 12B is a graph comparing relative insulin receptor binding of native insulin (measured at 1.5 hour at 4° C.) and the A19 IGF prodrug analog (IGF1YL: dK(e-acetyl),Sar) over time (0 hours, 9 hours and 95 hours) incubated in 20% plasma/PBS. As indicated by the data presented in the graph, increased activity is recovered from the A19 IGF prodrug analog sample as the prodrug form is converted to the active IGF1YL peptide.

### DETAILED DESCRIPTION

### Definitions

In describing and claiming the invention, the following terminology will be used in accordance with the definitions set forth below.

As used herein, the term “prodrug” is defined as any compound that undergoes chemical modification before exhibiting its pharmacological effects.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. For the purposes of the present disclosure designation of an amino acid without specifying its stereochemistry is intended to encompass either the L or D form of the amino acid, or a racemic mixture. However, in the instance where an amino acid is designated by its three letter code and includes a superscript number, the D form of the amino acid is specified by inclusion of a lower case d before the three letter code and superscript number (e.g., dLys⁻¹), wherein the designation lacking the lower case d (e.g., Lys⁻¹) is intended to specify the native L form of the amino acid. In this nomenclature, the inclusion of the superscript number designates the position of the amino acid in the IGF peptide sequence, wherein amino acids that are located within the IGF sequence are designated by positive superscript numbers numbered consecutively from the N-terminus. Additional amino acids linked to the IGF peptide either at the N-terminus or through a side chain are numbered starting with 0 and increasing in negative integer value as they are further removed from the IGF sequence. For example, the position of an amino acid within a dipeptide prodrug linked to the N-terminus of IGF is designated aa⁻¹-aa⁰-IGF wherein aa⁰ represents the carboxy terminal amino acid of the dipeptide and aa⁻¹ designates the amino terminal amino acid of the dipeptide.

As used herein the term “hydroxyl acid” refers to amino acids that have been modified to replace the alpha carbon amino group with a hydroxyl group.

As used herein the term “non-coded amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “dipeptide” is a compound formed by linkage of an alpha amino acid or an alpha hydroxyl acid to another amino acid, through a peptide bond.

As used herein the term “chemical cleavage” absent any further designation encompasses a non-enzymatic reaction that results in the breakage of a covalent chemical bond.

A “bioactive polypeptide” refers to polypeptides which are capable of exerting a biological effect in vitro and/or in vivo.

As used herein a general reference to a peptide is intended to encompass peptides that have modified amino and carboxy termini. For example, an amino acid sequence designating the standard amino acids is intended to encompass standard amino acids at the N- and C- terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, such as DPP-IV, and (v) increased potency at the IGF and/or insulin peptide receptors.

As used herein, an “alkylated” amino acid is an amino acid comprising an alkyl group which is non-native to a naturally-occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases, such as DPP-IV, and increased potency at the IGF and/or insulin receptors.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to maintaining glucose blood levels near normal levels and may include increasing or decreasing blood glucose levels depending on a given situation.

As used herein an “effective” amount or a “therapeutically effective amount” of an insulin analog refers to a nontoxic but sufficient amount of an insulin analog to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein the term “native insulin peptide” is intended to designate the 51 amino acid heterodimer comprising the A chain of SEQ ID NO: 1 and the B chain of SEQ ID NO: 2, as well as single-chain insulin analogs that comprise SEQ ID NOS: 1 and 2. The term “insulin peptide” as used herein, absent further descriptive language is intended to encompass the 51 amino acid heterodimer comprising the A chain of SEQ ID NO: 1 and the B chain of SEQ ID NO: 2, as well as single-chain insulin analogs thereof (including for example those disclosed in published international application WO96/34882 and U.S. Pat. No. 6,630,348, the disclosures of which are incorporated herein by reference), including heterodimers and single-chain analogs that comprise modified derivatives of the native A chain and/or B chain, including modification of the amino acid at position A19, B16 or B25 to a 4-amino phenylalanine or one or more amino acid substitutions at positions selected from A5, A8, A9, A10, A12, A14, A15, A17, A18, A21, B1, B2, B3, B4, B5, B9, B10, B13, B14, B17, B20, B21, B22, B23, B26, B27, B28, B29 and B30 or deletions of any or all of positions B1-4 and B26-30.

An “A19 insulin analog” is an insulin peptide that has a substitution of 4-amino phenylalanine or 4-methoxy phenylalanine for the native tyrosine residue at position 19 of the A chain of native insulin.

As used herein an “IGF^{B16B17} derivative peptide” is a generic term that comprising an A chain and B chain heterodimer, as well as single-chain insulin analogs thereof, wherein the A chain comprises the peptide sequence of SEQ ID NO: 19 and the B chain comprises the sequence of SEQ ID NO: 20 as well as derivatives of those sequences wherein the derivative of the A chain and/or B chain comprise 1-3 further amino acid substitutions, with the proviso that the A chain does not comprise the sequence of SEQ ID NO: 1 and/or the B chain does not comprise the sequence of SEQ ID NO: 2.

A “YL IGF analog” is a peptide comprising an IGF A chain of SEQ ID NO: 19 and an IGF B chain of SEQ ID NO: 9.

As used herein, the term “single-chain IGF^{B16B17} derivative peptide” encompasses a group of structurally-related proteins wherein IGF^{B16B17} derivative peptide A and B chains are covalently linked.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue. Throughout the application, all references to a particular amino acid position by letter and number (e.g. position A5) refer to the amino acid at that position of either the A chain (e.g. position A5) or the B chain (e.g. position B5) in the respective native human insulin A chain (SEQ ID NO: 1) or B chain (SEQ ID NO: 2), or the corresponding amino acid position in any analogs thereof. For example, a reference herein to “position B28” absent any further elaboration would mean the corresponding position B27 of the B chain of an insulin analog in which the first amino acid of SEQ ID NO: 2 has been deleted.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine

V. Large, aromatic residues:
Phe, Tyr, Trp, acetyl phenylalanine

As used herein the general term “polyethylene glycol chain” or “PEG chain”, refers to mixtures of condensation polymers of ethylene oxide and water, in a branched or straight chain, represented by the general formula H(OCH₂CH₂)ₙOH, wherein n is at least 9. Absent any further characterization, the term is intended to include polymers of ethylene glycol with an average total molecular weight selected from the range of 500 to 80,000 Daltons. “Polyethylene glycol chain” or “PEG chain” is used in combination with a numeric suffix to indicate the approximate average molecular weight thereof. For example, PEG-5,000 refers to polyethylene glycol chain having a total molecular weight average of about 5,000 Daltons.

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups.

As used herein an “IGF dimer” is a complex comprising two IGF^{B16B17} derivative peptides (each itself comprising an A chain and a B chain) covalently bound to one another via a linker. The term IGF dimer, when used absent any qualifying language, encompasses both IGF homodimers and IGF heterodimers. An IGF homodimer comprises two identical subunits, whereas an IGF heterodimer comprises two subunits that differ, although the two subunits are substantially similar to one another.

The term “C₁-Cₙ alkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-butyl, sec-butyl, tert-butyl, pentyl, hexyl and the like.

The terms “C₂-Cₙ alkenyl” wherein n can be from 2 through 6, as used herein, represents an olefinically unsaturated branched or linear group having from 2 to the specified number of carbon atoms and at least one double bond. Examples of such groups include, but are not limited to, 1-propenyl, 2-propenyl (—CH₂—CH═CH₂), 1,3-butadienyl, (—CH═CHCH═CH₂), 1-butenyl(—CH═CHCH₂CH₃), hexenyl, pentenyl, and the like.

The term “C₂-Cₙ alkynyl” wherein n can be from 2 to 6, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. The size of the aryl ring and the presence of substituents or linking groups are indicated by designating the number of carbons present. For example, the term “(C₁-C₃ alkyl)(C₆-C₁₀ aryl)” refers to a 5 to 10 membered aryl that is attached to a parent moiety via a one to three membered alkyl chain.

The term “heteroaryl” as used herein refers to a mono- or bi-cyclic ring system containing one or two aromatic rings and containing at least one nitrogen, oxygen, or sulfur atom in an aromatic ring. The size of the heteroaryl ring and the presence of substituents or linking groups are indicated by designating the number of carbons present. For example, the term “(C₁-Cₙ alkyl)(C₅-C₆ heteroaryl)” refers to a 5 or 6 membered heteroaryl that is attached to a parent moiety via a one to “n” membered alkyl chain.

As used herein, the term “halo” refers to one or more members of the group consisting of fluorine, chlorine, bromine, and iodine.

As used herein the term “patient” without further designation is intended to encompass any warm blooded vertebrate domesticated animal (including for example, but not limited to livestock, horses, cats, dogs and other pets) and humans.

### Embodiments

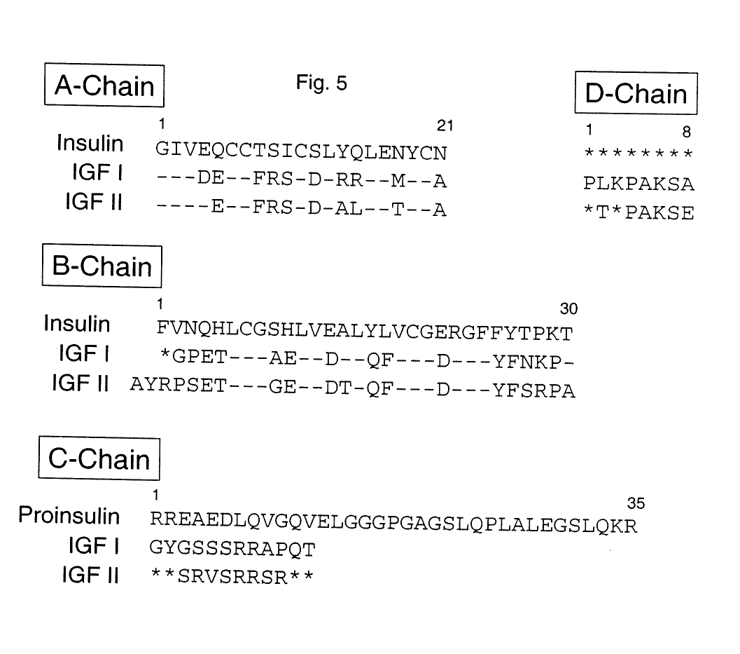

As shown by the alignment of the human insulin and insulin-like growth factors I and II (IGF I and IGF II), these three peptides share a high level of sequence identity (see FIG. 5). As disclosed herein the B16 tyrosine of native insulin has been found to be an amino acid of great importance for high affinity insulin agonism. More particularly, applicants have discovered that derivatives of IGF I and IGF II that comprise a substitution of a tyrosine leucine dipeptide for the native IGF amino acids at positions corresponding to B16 and B17 of native insulin have a tenfold increase in potency at the insulin receptor. Thus, the remaining differences in the relative amino acid sequence of insulin and IGFs appears to be of lesser importance to high affinity interaction of insulin-like ligands with the insulin receptor.

In accordance with one embodiment an IGF^{B16B17} derivative peptide is provided comprising an A chain of IGF I (SEQ ID NO: 5) or IGF II (SEQ ID NO: 7) and a B chain of IGF I (SEQ ID NO: 6) or IGF II (SEQ ID NO: 8), wherein the native IGF amino acids at positions corresponding to positions 16 and 17 of the native insulin B chain sequence have been replaced with tyrosine and leucine, respectively. In addition, the IGF^{B16B17} derivative peptides disclosed herein may also comprise further modifications to the A chain and B chain, wherein such modifications either further enhance the activity at the insulin receptor and/or decrease activity at the IGF-1 receptor. Additional modifications include, for example, modification of the amino acids at one or more of positions A19, B16 or B25 (relative to the native insulin A and B chains) to a 4-amino phenylalanine or one or more amino acid substitutions at positions selected from A5, A8, A9, A10, A14, A15, A17, A18, A21, B1, B2, B3, B4, B5, B9, B10, B13, B14, B20, B21, B22, B23, B26, B27, B28, B29, and B30 (relative to the native A and B chains of insulin) or deletions of any or all of positions B1-4 and B26-30, provided that the IGF^{B16B17} derivative peptide does not comprise the sequences of SEQ ID NO: 1 and SEQ ID NO: 2. In one embodiment the substitutions at positions selected from A5, A8, A9, A10, A14, A15, A17, A18, A21, B1, B2, B3, B4, B5, B9, B10, B13, B14, B20, B21, B22, B23, B26, B27, B28, B29, and B30 are conservative amino acid substitutions. In one embodiment the IGF^{B16B17} derivative peptide comprises an A chain peptide sequence of SEQ ID NO: 19 and a B chain peptide sequence of SEQ ID NO: 17 as well as derivatives of those sequences wherein the derivative of the A chain and B chain each comprise 1-3 further amino acid substitutions, with the proviso that the A chain does not comprise the sequence of SEQ ID NO: 1 and/or the B chain does not comprise the sequence of SEQ ID NO: 2.

In one embodiment the IGF^{B16B17} derivative peptides exhibit 70%, 80%, 90%, 95%, 100% or greater activity at the insulin receptor relative to native insulin. In one embodiment the IGF^{B16B17} derivative peptides retain activity at the IGF receptor, but in an alternative embodiment the IGF^{B16B17} derivative peptide has high activity for the insulin receptor relative to native insulin (e.g., 90%, 95%, 100% or greater activity), but substantially reduced activity (e.g., less than 20%, less than 10% or less than 5%) at the IGF I receptor relative to native IGF I.

In accordance with one embodiment, the IGF^{B16B17} derivative peptides disclosed herein are used as full and super-potent insulin agonists and thus have utility in any previously disclosed use for insulin. Additional virtues of the presently disclosed IGF^{B16B17} derivative peptides include, but are not limited to relative ease of synthesis, development of co-agonists for insulin and IGF 1 receptors, and potentially selective insulin receptor isoform specific analogs.

In accordance with one embodiment a polypeptide comprising the sequence X₂₅LCGX₂₉X₃₀LVX₃₃X₃₄LYLVCGDX₄₂GFY—R₁₄ (SEQ ID NO: 9) is provided, wherein

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine; and

X₄₂ is selected from the group consisting of alanine and arginine.

In accordance with one embodiment this peptide is linked to a second peptide having the sequence GIVDECCX₈X₉SCDLX₁₄X₁₅LEX₁₈YCX₂₁—R₁₃ (SEQ ID NO: 10) wherein

X₈ is histidine or phenylalanine;

X₉ and X₁₄ are independently selected from arginine or alanine;

X₁₅ is arginine or leucine;

X₁₈ is methionine, asparagine or threonine;

X₂₁ is alanine, glycine or asparagine; and

R₁₃ and R₁₄ are independently COOH or CONH₂. In one embodiment the two peptides of SEQ ID NO: 9 and SEQ ID NO: 10 are linked to one another by intermolecular disulfide bonds to form an IGF analog heterodimer. In an alternative embodiment the N-terminus of one peptide is linked to the C-terminus of the other peptides to form a single chain IGF^{B16B17} derivative peptide. More particularly, in one embodiment the carboxy terminus of SEQ ID NO: 9 is linked to the N-terminus of the peptide of SEQ ID NO: 10 through a peptide bond.

The IGF^{B16B17} derivative peptides disclosed herein may comprise additional modifications relative to the native IGF sequence besides the substitution of the amino acids at position B16 and B17. For example, IGF^{B16B17} derivative peptides may comprise an IGF A chain and an IGF B chain, wherein the A chain comprises the sequence GIVDECCFRSCDLRRLEMYCA (SEQ ID NO: 5) or GIVEECCFRSCDLALLETYCA (SEQ ID NO: 7) and the B chain comprises the sequence GPETLCGAELVDALYLVCGDRGFYFNKPT (SEQ ID NO: 11) or AYRPSETLCGGELVDTLYLVCGDRGFYFSRPA (SEQ ID NO: 12), wherein those sequences are further modified to comprise one or more additional amino acid substitutions at positions corresponding to native insulin positions (see peptide alignment shown in FIG. 5) selected from A5, A8, A9, A10, A14, A15, A17, A18, A21, B1, B2, B3, B4, B5, B9, B10, B13, B14, B20, B22, B23, B26, B27, B28, B29, and B30, with the proviso that the A chain does not comprise the sequence of SEQ ID NO: 1 and the B chain does not comprise the sequence of SEQ ID NO: 2. In one embodiment the amino acid substitutions are conservative amino acid substitutions. Suitable amino acid substitutions at these positions that do not adversely impact insulin's desired activities are known to those skilled in the art, as demonstrated, for example, in Mayer, et al., Insulin Structure and Function, Biopolymers. 2007; 88(5):687-713, the disclosure of which is incorporated herein by reference. Such modifications are also believed to be suitable for the IGF^{B16B17} derivative peptides disclosed herein. In accordance with one embodiment IGF^{B16B17} derivative peptides may comprise an IGF A chain and an IGF B chain, wherein the A chain comprises an amino acid sequence that shares at least 70% sequence identity (e.g., 70%, 75%, 80%, 85%, 90%, 95%) with at least one of GIVDECCFRSCDLRRLEMYCA (SEQ ID NO: 5) or GIVEECCFRSCDLALLETYCA (SEQ ID NO: 7) and the B chain comprises an amino acid sequence that shares at least 60% sequence identity (e.g., 60%, 65%, 70%, 75%, 80%, 85%, 90%, 95%) with at least one of the sequence GPETLCGAELVDALYLVCGDRGFYFNKPT (SEQ ID NO: 11) or AYRPSETLCGGELVDTLYLVCGDRGFYFSRPA (SEQ ID NO: 12). In one embodiment the IGF^{B16B17} derivative peptides disclosed herein comprise a C-terminal amide or ester in place of a C-terminal carboxylate on the A chain and/or B chain.

In accordance with one embodiment an IGF^{B16B17} derivative peptide is provided comprising an A chain having the sequence GIVX₄X₅CCX₈X₉X₁₀CX₁₂LX₁₄X₁₅LEX₁₈X₁₉CX₂₁—R₁₃ (SEQ ID NO: 82) and a B chain having the sequence R₂₂—X₂₅LCGX₂₉X₃₀LVX₃₃X₃₄LYLVCGX₄₁X₄₂GFX₄₅R₄₇—R₄₈—R₄₉—R₁₄ (SEQ ID NO: 67), wherein

X₄ is glutamic acid or aspartic acid;

X₅ is glutamic acid or glutamine;

X₈ is histidine, threonine or phenylalanine;

X₉ is serine, ornathine, arginine or alanine;

X₁₀ is serine or isoleucine;

X₁₂ is serine or aspartic acid;

X₁₄ are independently selected from tyrosine, ornathine, arginine or alanine;

X₁₅ is glutamine, ornathine, arginine, alanine or leucine;

X₁₈ is methionine, asparagine or threonine;

X₁₉ is tyrosine, or 4-amino phenylalanine;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid and aspartic acid;

X₄₂ is selected from the group consisting of alanine, ornithine and arginine;

X₄₅ is phenylalanine or tyrosine;

R₁₃ and R₁₄ are independently COOH or CONH₂;

R₂₂ is selected from the group consisting of AYRPSE (SEQ ID NO: 14), FGPE (SEQ ID NO: 68), the tripeptide glycine-proline-glutamic acid, the dipeptide proline-glutamic acid, glutamic acid and an N-terminal amine;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a proline-arginine dipeptide, a lysine-proline dipeptide, or a proline-lysine dipeptide;

R₄₉ is threonine or alanine; and R₁₃ and R₁₄ are independently selected from COOH and CONH₂, with the proviso that the B chain is not a native insulin B chain sequence (e.g., not SEQ ID NO: 2).

In accordance with one embodiment an IGF^{B16B17} derivative peptide is provided comprising an A chain comprising the sequence GIVX₄X₅CCX₈X₉X₁₀CX₁₂LX₁₄X₁₅LEX₁₈X₁₉CX₂₁—R₁₃ (SEQ ID NO: 82) and a B chain comprising the sequence R₂₂—X₂₅LCGX₂₉X₃₀LVX₃₃X₃₄LYLVCGDX₄₂GFX₄₅R₄₇—R₄₈—R₄₉—R₁₄ (SEQ ID NO: 67), wherein

X₄ is glutamic acid or aspartic acid;

X₅ is glutamic acid or glutamine;

X₈ is histidine, threonine or phenylalanine;

X₉ is serine, arginine or alanine;

X₁₀ is serine or isoleucine;

X₁₂ is serine or aspartic acid;

X₁₄ are independently selected from tyrosine, arginine or alanine;

X₁₅ is glutamine, arginine, alanine or leucine;

X₁₈ is methionine, asparagine or threonine;

X₁₉ is tyrosine, or 4-amino phenylalanine;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of ornathine and arginine;

X₄₅ is phenylalanine or tyrosine;

R₁₃ and R₁₄ are independently COOH or CONH₂;

R₂₂ is selected from the group consisting of AYRPSE (SEQ ID NO: 14), the tripeptide glycine-proline-glutamic acid, the dipeptide proline-glutamic acid, glutamic acid and an N-terminal amine;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a proline-arginine dipeptide, a lysine-proline dipeptide, or a proline-lysine dipeptide;

R₄₉ is threonine or alanine; and R₁₃ and R₁₄ are independently selected from COOH and CONH₂, with the proviso that the B chain is not a native insulin B chain sequence (e.g., not SEQ ID NO: 2).

In accordance with one embodiment an IGF^{B16B17} derivative peptide is provided comprising an A chain having the sequence GIVX₄ECCX₈X₉SCDLX₁₄X₁₅LEX₁₈X₁₉CX₂₁ (SEQ ID NO: 19) and a B chain comprising the sequence X₂₅LCGX₂₉ELVDX₃₄LYLVCGDX₄₂GFY (SEQ ID NO: 65) or a derivative of SEQ ID NO: 65 modified to have 1 to 3 amino acid substitutions at positions B4, B5, B8, B9, B15, B16, B18, B21, B22 and B23 relative to SEQ ID NO: 65. In one embodiment the 1 to 3 amino acid substitutions are conservative amino acid substitutions. In one embodiment the B chain of SEQ ID NO: 65 is modified by one to two amino acid substitutions, at positions corresponding to native insulin positions, selected from the group consisting of serine at B9, histidine at B10, glutamic acid at B13, alanine at B14 and asparagine at B21.

In accordance with one embodiment an IGF^{B16B17} derivative peptide is provided comprising an A chain comprising the sequence GIVX₄ECCX₈X₉SCDLX₁₄X₁₅LEX₁₈X₁₉CX₂₁—R₁₃ (SEQ ID NO: 19) and a B chain comprising the sequence X₂₅LCGX₂₉X₃₀LVX₃₃X₃₄LYLVCGDX₄₂GFY—R₁₄ (SEQ ID NO: 9), wherein

X₄ is aspartic acid or glutamic acid;

X₈ is histidine or phenylalanine;

X₉ and X₁₄ are independently selected from arginine, ornathine or alanine;

X₁₅ is arginine, ornathine or leucine;

X₁₈ is methionine, asparagine or threonine;

X₁₉ is tyrosine, 4-methoxy-phenylalanine or 4-amino-phenylalanine;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of alanine ornathine and arginine; and R₁₃ and R₁₄ are independently COOH or CONH₂. In one embodiment R₁₃ is COOH and R₁₄ is CONH₂. In one embodiment X₁₉ is tyrosine. In a further embodiment X₁₉ is tyrosine, X₄ is aspartic acid and X₂₉ is alanine. In one embodiment the B chain comprises the sequence R₂₂—X₂₅LCGX₂₉X₃₀LVX₃₃X₃₄LYLVCGDX₄₂GFY—R₄₇—R₄₈—R₄₉—R₁₄ (SEQ ID NO: 9), wherein R₂₂ is selected from the group consisting of the peptide of AYRPSE (SEQ ID NO: 14), a glycine-proline-glutamic acid tripeptide, a proline-glutamic acid dipeptide, glutamic acid and an N-terminal amine (i.e., no additional amino acid residue), R₄₇ is a phenylalanine-asparagine dipeptide, a phenylalanine-serine dipeptide or a tyrosine-threonine dipeptide, R₄₈ is an aspartate-lysine dipeptide, an arginine-proline dipeptide, a lysine-proline dipeptide, or a proline-lysine dipeptide, and R₄₉ is threonine or alanine; and R₁₃ and R₁₄ are independently COOH or CONH₂.

In accordance with one embodiment an IGF^{B16B17} derivative peptide is provided comprising an A chain comprising the sequence GIVX₄ECCX₈X₉SCDLX₁₄X₁₅LEX₁₈X₁₉CX₂₁—R₁₃ (SEQ ID NO: 19) and a B chain comprising the sequence X₂₅LCGX₂₉ELVDX₃₄LYLVCGDX₄₂GFY (SEQ ID NO: 65), wherein

X₄ is aspartic acid or glutamic acid;

X₈ is phenylalanine or histidine;

X₉ is arginine, ornathine or alanine;

X₁₄ is arginine or alanine;

X₁₅ is arginine or leucine;

X₁₈ is methionine or threonine;

X₁₉ is tyrosine, 4-methoxy-phenylalanine or 4-amino-phenylalanine;

X₂₁ is alanine, glycine or asparagine;

X₂₅ is histidine or threonine;

X₂₉ is selected from the group consisting of alanine and glycine;

X₃₄ is selected from the group consisting of alanine and threonine; and

X₄₂ is selected from the group consisting of alanine ornathine and arginine; and R₁₃ is COOH or CONH₂.

In one embodiment an IGF^{B16B17} derivative peptide is provided comprising an A chain comprising the sequence GIVDECCX₈X₉SCDLRRLEMX₁₉CX₂₁—R₁₃ (SEQ ID NO: 19) and a B chain comprising the sequence X₂₅LCGAX₃₀LVDALYLVCGDX₄₂GFY (SEQ ID NO: 18), wherein

X₈ is phenylalanine or histidine;

X₉ is arginine, ornathine or alanine;

X₁₉ is tyrosine, 4-methoxy-phenylalanine or 4-amino-phenylalanine;

X₂₁ is alanine or asparagine;

X₂₅ is histidine or threonine;

X₃₀ is selected from the group consisting of histidine, aspartic acid, glutamic acid, homocysteic acid and cysteic acid;

X₄₂ is selected from the group consisting of alanine ornathine and arginine; and R₁₃ is COOH or CONH₂. In one embodiment R₁₃ is COOH and the carboxy terminal amino acid of the B peptide has an amide (CONH₂) in place of the natural alpha carbon carboxy group. In one embodiment X₁₉ is tyrosine. In one embodiment the B chain comprises the sequence R₂₂—X₂₅LCGAX₃₀LVDALYLVCGDX₄₂GFY—R₄₇—R₄₈—R₄₉—R₁₄ (SEQ ID NO: 18), wherein R₂₂ is selected from the group consisting of the peptide of AYRPSE (SEQ ID NO: 14), a glycine-proline-glutamic acid tripeptide, a proline-glutamic acid dipeptide, glutamic acid and an N-terminal amine, X₃₀ is selected from the group consisting of aspartic acid, glutamic acid, homocysteic acid and cysteic acid; X₄₂ is selected from the group consisting of alanine, ornathine and arginine; R₄₇ is a phenylalanine-asparagine dipeptide, a phenylalanine-serine dipeptide or a tyrosine-threonine dipeptide, R₄₈ is an aspartate-lysine dipeptide, an arginine-proline dipeptide, a proline-arginine dipeptide, a lysine-proline dipeptide, or a proline-lysine dipeptide, and R₄₉ is threonine or alanine; and R₁₄ is COOH or CONH₂. In one embodiment X₃₀ is glutamic acid and in a further embodiment X₃₀ is glutamic acid and X₄₂ is arginine.

In a further embodiment the IGF^{B16B17} derivative peptide comprises an A chain having the sequence GIVDECCX₈X₉SCDLX₁₄X₁₅LEX₁₈X₁₉CX₂₁—R₁₃ (SEQ ID NO: 13) and a B chain having the sequence of R₂₂—X₂₅LCGX₂₉X₃₀LVX₃₃X₃₄LYLVCGDX₄₂GFY—R₄₇—R₄₈—R₄₉—R₁₄ (SEQ ID NO: 9) wherein

X₈ is histidine or phenylalanine;

X₉ and X₁₄ are independently selected from arginine, ornathine or alanine;

X₁₅ is arginine, ornathine or leucine;

X₁₈ is methionine, asparagine or threonine;

X₁₉ is tyrosine or 4-amino-phenylalanine;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of alanine, ornathine and arginine;

R₁₃ and R₁₄ are independently COOH or CONH₂;

R₂₂ is selected from the group consisting of AYRPSE (SEQ ID NO: 14), a glycine-proline-glutamic acid tripeptide, a proline-glutamic acid dipeptide, glutamic acid and an N-terminal amine;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a lysine-proline dipeptide, or a proline-lysine dipeptide; and

R₄₉ is threonine or alanine; and R₁₃ and R₁₄ are independently COOH or CONH₂.

In one embodiment an IGF^{B16B17} derivative peptide is provided comprising an A chain having the sequence GIVDECCX₈X₉SCDLX₁₄X₁₅LEX₁₈YCX₂₁—R₁₃ (SEQ ID NO: 10) and a B chain comprising the sequence X₂₅LCGAX₃₀LVDALYLVCGDX₄₂GFYFN (SEQ ID NO: 15), wherein

X₈ is phenylalanine or histidine;

X₉ and X₁₄ are independently selected from arginine or alanine;

X₁₅ is arginine or leucine;

X₁₈ is methionine, asparagine or threonine;

X₂₁ is alanine, glycine or asparagine;

X₂₅ is histidine or threonine;

X₃₀ is glutamic acid or aspartic acid;

X₄₂ is arginine, alanine or ornathine;

R₁₃ and R₁₄ are independently COOH or CONH₂.

In accordance with one embodiment an IGF^{B16B17} derivative peptide is provided comprising an A chain having the sequence GIVDECCX₈X₉SCDLRRLEMYCX₂₁—R₁₃ (SEQ ID NO: 16) and a B chain having the sequence R₂₂—X₂₅LCGAX₃₀LVDALYLVCGDX₄₂GFYFN—R₄₈—R₄₉—R₁₄ (SEQ ID NO: 15), wherein

X₈ is histidine or phenylalanine;

X₉ is arginine or alanine;

X₂₁ is alanine, glycine or asparagine;

X₂₅ is histidine or threonine;

X₃₀ is selected from the group consisting of histidine, aspartic acid, glutamic acid, homocysteic acid and cysteic acid;

X₄₂ is selected from the group consisting of alanine and arginine;

R₂₂ is selected from the group consisting of a glycine-proline-glutamic acid tripeptide, a proline-glutamic acid dipeptide, glutamic acid and an N-terminal amine;

R₄₈ is an aspartate-lysine dipeptide, an arginine-proline dipeptide, a lysine-proline dipeptide, or a proline-lysine dipeptide;

R₄₉ is threonine;

R₁₃ is COOH and R₁₄ is CONH₂.

In a further embodiment, an IGF/insulin co-agonist is provided comprising an A chain having the sequence GIVDECCX₈X₉SCDLRRLEMYCX₂₁—R₁₃ (SEQ ID NO: 16) and a B chain having the sequence R₂₂—X₂₅LCGAX₃₀LVDALYLVCGDRGFYFNKPT-R₁₄ (SEQ ID NO: 17), wherein

X₈ is histidine or phenylalanine;

X₉ is arginine or alanine;

X₂₁ is alanine, glycine or asparagine;

R₂₂ is selected from the group consisting of a glycine-proline-glutamic acid tripeptide, a proline-glutamic acid dipeptide, glutamic acid and an N-terminal amine;

X₂₅ is histidine or threonine;

X₃₀ is selected from the group consisting of aspartic acid and glutamic acid;

R₁₃ is COOH and R₁₄ is CONH₂.

In one embodiment an IGF^{B16B17} derivative peptide having high specificity for the insulin receptor is provided wherein the peptide comprises an A chain having the sequence GIVDECCX₈X₉SCDLRRLEMYCX₂₁—R₁₃ (SEQ ID NO: 16) and a B chain comprising the sequence R₂₂—X₂₅LCGAX₃₀LVDALYLVCGDX₄₂GFY (SEQ ID NO: 18), wherein

X₈ is histidine or phenylalanine;

X₉ is arginine or alanine;

X₂₁ is alanine, glycine or asparagine;

R₂₂ is selected from the group consisting of a glycine-proline-glutamic acid tripeptide, a proline-glutamic acid dipeptide, glutamic acid and an N-terminal amine;

X₂₅ is histidine or threonine;

X₃₀ is selected from the group consisting of aspartic acid and glutamic acid;

X₄₂ is arginine, alanine or ornathine;

R₁₃ is COOH and the carboxy terminal amino acid of the B chain has an amide (CONH₂) in place of the native alpha carbon carboxylic acid. In one embodiment an IGF^{B16B17} derivative peptide having high specificity for the insulin receptor is provided wherein the peptide comprises an A chain having the sequence GIVDECCFRSCDLRRLEMX₁₉CA-R₁₃ (SEQ ID NO: 22) and a B chain having the sequence R₂₂-TLCGAELVDALYLVCGDRGFYFNKPT-R₁₄ (SEQ ID NO: 64), wherein

X₁₉ is tyrosine or 4-amino-phenylalanine;

R₂₂ is selected from the group consisting of a glycine-proline-glutamic acid tripeptide, a proline-glutamic acid dipeptide, glutamic acid and an N-terminal amine; and

R₁₃ and R₁₄ are independently COOH or CONH₂. In one embodiment an IGF^{B16B17} derivative peptide having high specificity for the insulin receptor is provided wherein the peptide comprises an A chain comprising the sequence GIVDECCFRSCDLRRLEMYCA-R₁₃ (SEQ ID NO: 22) and a B chain comprising the sequence GPETLCGAELVDALYLVCGDRGFYFNKPT-R₁₄ (SEQ ID NO: 11), wherein R₁₃ and R₁₄ are independently COOH or CONH₂. In another embodiment an IGF^{B16B17} derivative peptide having high specificity for the insulin receptor is provided wherein the peptide comprises an A chain comprising the sequence GIVDECCX₈X₉SCDLRRLEMX₁₉CA-R₁₃ (SEQ ID NO: 22) and a B chain comprising the sequence GPEX₂₅LCGAELVDALYLVCGDX₄₂GFY—R₁₄ (SEQ ID NO: 11), wherein

X₈ is histidine or phenylalanine;

X₉ is arginine or alanine;

X₁₉ is tyrosine or 4-amino-phenylalanine;

X₂₅ is histidine or threonine;

X₄₂ is arginine, alanine or ornathine;

R₁₃ and R₁₄ are independently COOH or CONH₂.

The IGF^{B16B17} derivative peptides disclosed herein (including both active forms as well as prodrug and depot formulations) may be part of a dimer, trimer or higher order multimer comprising at least two, three, or more peptides bound via a linker, wherein at least one or both peptides is a the IGF^{B16B17} derivative peptide. The dimer may be a homodimer or heterodimer, comprising peptides selected from the group consisting of native insulin, native IGF-1, native IGF-II, an insulin analog peptide and IGF^{B16B17} derivative peptides. In some embodiments, the linker is selected from the group consisting of a bifunctional thiol crosslinker and a bi-functional amine crosslinker. In certain embodiments, the linker is PEG, e.g., a 5 kDa PEG, 20 kDa PEG. In some embodiments, the linker is a disulfide bond.

For example, each monomer of the dimer may comprise a Cys residue (e.g., a terminal or internally positioned Cys) and the sulfur atom of each Cys residue participates in the formation of the disulfide bond. Each monomer of the dimer represents a heterodimer of an A and B chain linked to one naother by disulfide bonds or prepared as single chain peptides. In some aspects of the invention, the monomers are connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer are attached together in a “tail-to-tail” orientation in which the C-terminal amino acids of each monomer are attached together. A conjugate moiety may be covalently linked to any of the IGF^{B16B17} derivative peptides described herein, including a dimer, trimer or higher order multimer.

Prodrug Derivatives of IFG Insulin Analogs

The present disclosure also encompasses prodrug derivatives of the IGF^{B16B17} derivative peptides disclosed herein. Advantageously the prodrug formulations improve the therapeutic index of the underlying peptide and delay onset of action and enhance the half life of the IGF^{B16B17} derivative peptide. The disclosed prodrug chemistry can be chemically conjugated to active site amines to form amides that revert to the parent amine upon diketopiperazine formation and release of the prodrug element. This novel biologically friendly prodrug chemistry spontaneously degrades under physiological conditions (e.g. pH of about 7, at 37° C. in an aqueous environment) and is not reliant on enzymatic degradation. The duration of the prodrug derivative is determined by the selection of the dipeptide prodrug sequence, and thus allows for flexibility in prodrug formulation.

In one embodiment a prodrug is provided having a non-enzymatic activation half time (t½) of between 1-100 hrs under physiological conditions. Physiological conditions as disclosed herein are intended to include a temperature of about 35 to 40° C. and a pH of about 7.0 to about 7.4 and more typically include a pH of 7.2 to 7.4 and a temperature of 36 to 38° C. in an aqueous environment. In one embodiment a dipeptide, capable of undergoing diketopiperazine formation under physiological conditions, is covalently linked through an amide linkage to the IGF^{B16B17} derivative peptide.

Advantageously, the rate of cleavage, and thus activation of the prodrug, depends on the structure and stereochemistry of the dipeptide pro-moiety and also on the strength of the nucleophile. The prodrugs disclosed herein will ultimately be chemically converted to structures that can be recognized by the insulin/IGF receptor, wherein the speed of this chemical conversion will determine the time of onset and duration of in vivo biological action. The prodrug chemistry disclosed in this application relies upon an intramolecular chemical reaction that is not dependent upon additional chemical additives, or enzymes. The speed of conversion is controlled by the chemical nature of the dipeptide substituent and its cleavage under physiological conditions. Since physiological pH and temperature are tightly regulated within a highly defined range, the speed of conversion from prodrug to drug will exhibit high intra and interpatient reproducibility.

As disclosed herein prodrugs are provided wherein the IGF^{B16B17} derivative peptides have extended half lives of at least 1 hour, and more typically greater than 20 hours but less than 100 hours, and are converted to the active form at physiological conditions through a non-enzymatic reaction driven by inherent chemical instability. In one embodiment the a non-enzymatic activation t½ time of the prodrug is between 1-100 hrs, and more typically between 12 and 72 hours, and in one embodiment the t½ is between 24-48 hrs as measured by incubating the prodrug in a phosphate buffer solution (e.g., PBS) at 37° C. and pH of 7.2. In one embodiment the half life of the prodrugs is about 1, 8, 12, 20, 24, 48 or 72 hours. In one embodiment the half life of the prodrugs is about 100 hours or greater including half lives of up to about 168, 336, 504, 672 or 720 hours, and are converted to the active form at physiological conditions through a non-enzymatic reaction driven by inherent chemical instability. The half lives of the various prodrugs are calculated by using the formula t_{1/2}=0.693/k, where ‘k’ is the first order rate constant for the degradation of the prodrug. In one embodiment, activation of the prodrug occurs after cleavage of an amide bond linked dipeptide, and formation of a diketopiperazine or diketomorpholine, and the active IGF^{B16B17} derivative peptide.

In another embodiment, the dipeptide prodrug element is covalently bound to the IGF^{B16B17} derivative peptide via an amide linkage, and the dipeptide further comprises a depot polymer linked to dipeptide. In one embodiment two or more depot polymers are linked to a single dipeptide element. In one embodiment the depot polymer is linked to the side chain of one of the amino acids comprising the dipeptide prodrug element. The depot polymer is selected to be biocompatible and of sufficient size that the IGF^{B16B17} derivative peptide modified by covalent attachment of the dipeptide remains sequestered at an injection site and/or incapable of interacting with its corresponding receptor upon administration to a patient. Subsequent cleavage of the dipeptide releases the IGF^{B16B17} derivative peptide to interact with its intended target. The depot bearing dipeptide element can be linked to the IGF^{B16B17} derivative peptide via an amide bond through any convenient amine group of the IGF^{B16B17} derivative peptide, including an N-terminal amine or an amine bearing side chain of an internal natural or synthetic amino acid of the IGF^{B16B17} derivative peptide.

In accordance with one embodiment the depot polymer is selected from biocompatible polymers known to those skilled in the art. The depot polymers typically have a size selected from a range of about 20,000 to 120,000 Daltons. In one embodiment the depot polymer has a size selected from a range of about 40,000 to 100,000 or about 40,000 to 80,000 Daltons. In one embodiment the depot polymer has a size of about 40,000, 50,000, 60,000, 70,000 or 80,000 Daltons. Suitable depot polymers include but are not limited to dextrans, polylactides, polyglycolides, caprolactone-based polymers, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof, and biodegradable polymers and their copolymers including caprolactone-based polymers, polycaprolactones and copolymers which include polybutylene terephthalate. In one embodiment the depot polymer is selected from the group consisting of polyethylene glycol, dextran, polylactic acid, polyglycolic acid and a copolymer of lactic acid and glycolic acid, and in one specific embodiment the depot polymer is polyethylene glycol. In one embodiment the depot polymer is polyethylene glycol and the combined molecular weight of depot polymer(s) linked to the dipeptide element is about 40,000 to 80,000 Daltons.

Specific dipeptides composed of natural or synthetic amino acids have been identified that facilitate intramolecular decomposition under physiological conditions to release the active IGF^{B16B17} derivative peptide. The dipeptide can be linked (via an amide bond) to an amino group present on the IGF^{B16B17} derivative peptide, or an amino group introduced into the IGF^{B16B17} derivative peptide by modification of the peptide sequence. In one embodiment the dipeptide structure is selected to resist cleavage by peptidases present in mammalian sera, including for example dipeptidyl peptidase IV (DPP-IV). Accordingly, in one embodiment the rate of cleavage of the dipeptide prodrug element from the bioactive peptide is not substantially enhanced (e.g., greater than 2×) when the reaction is conducted using physiological conditions in the presence of serum proteases relative to conducting the reaction in the absence of the proteases. Thus the cleavage half-life of the dipeptide prodrug element from the IGF^{B16B17} derivative peptide (in PBS under physiological conditions) is not more than two, three, four or five fold the cleavage half-life of the dipeptide prodrug element from the IGF^{B16B17} derivative peptide in a solution comprising a DPP-IV protease. In one embodiment the solution comprising a DPP-IV protease is serum, more particularly mammalian serum, including human serum.

In accordance with one embodiment the dipeptide prodrug element comprises the structure U—O, wherein U is an amino acid or a hydroxyl acid and O is an N-alkylated amino acid. The structure of U—O is selected, in one embodiment, wherein chemical cleavage of U—O from the IGF^{B16B17} derivative peptide is at least about 90% complete within about 1 to about 720 hours in PBS under physiological conditions. In one embodiment the chemical cleavage half-life (t_{1/2}) of U—O from the IGF^{B16B17} derivative peptide is at least about 1 hour to about 1 week in PBS under physiological conditions. In one embodiment U, O, or the amino acid of the IGF^{B16B17} derivative peptide to which U—O is linked is a non-coded amino acid. In some embodiments U and/or O is an amino acid in the D stereoisomer configuration. In some exemplary embodiments, U is an amino acid in the D stereoisomer configuration and O is an amino acid in the L stereoisomer configuration. In some exemplary embodiments, U is an amino acid in the L stereoisomer configuration and O is an amino acid in the D stereoisomer configuration. In some exemplary embodiments, U is an amino acid in the D stereoisomer configuration and O is an amino acid in the D stereoisomer configuration. In one embodiment O is an N-alkylated amino acid but is not proline. In one embodiment the N-alkylated group of amino acid O is a C₁-C₁₈ alkyl, and in one embodiment the N-alkylated group is C₁-C₆ alkyl.

In one embodiment one or more dipeptide elements are linked to the IGF^{B16B17} derivative peptide through an amide bond formed through one or more amino groups selected from the N-terminal amino group of the A or B chain, or the side chain amino group of an amino acid present in the IGF^{B16B17} derivative peptide. In one embodiment the IGF^{B16B17} derivative peptide comprises two dipeptide elements, wherein the dipeptide elements are optionally pegylated, alkylated, acylated or linked to a depot polymer. In accordance with one embodiment the dipeptide extension is covalently linked to an IGF^{B16B17} derivative peptide through the side chain amine of a lysine residue that resides at or near the active site. In one embodiment the dipeptide extension is attached through a synthetic amino acid or a modified amino acid, wherein the synthetic amino acid or modified amino acid exhibits a functional group suitable for covalent attachment of the dipeptide extension (e.g., the aromatic amine of amino-phenylalanine). In accordance with one embodiment one or more dipeptide elements are linked to the IGF^{B16B17} derivative peptide at an amino group selected from the N-terminal amino group of the A or B chain, or the side chain amino group of an aromatic amine of a 4-amino-phenylalanine residue present at a position corresponding to position A19, B16 or B25 of native insulin.

The dipeptide prodrug element is designed to spontaneously cleave its amide linkage to the insulin analog under physiological conditions and in the absence of enzymatic activity. In one embodiment the N-terminal amino acid of the dipeptide extension comprises a C-alkylated amino acid (e.g. amino isobutyric acid). In one embodiment the C-terminal amino acid of the dipeptide comprises an N-alkylated amino acid (e.g., proline or N-methyl glycine). In one embodiment the dipeptide comprises the sequence of an N-terminal C-alkylated amino acid followed by an N-alkylated amino acid.

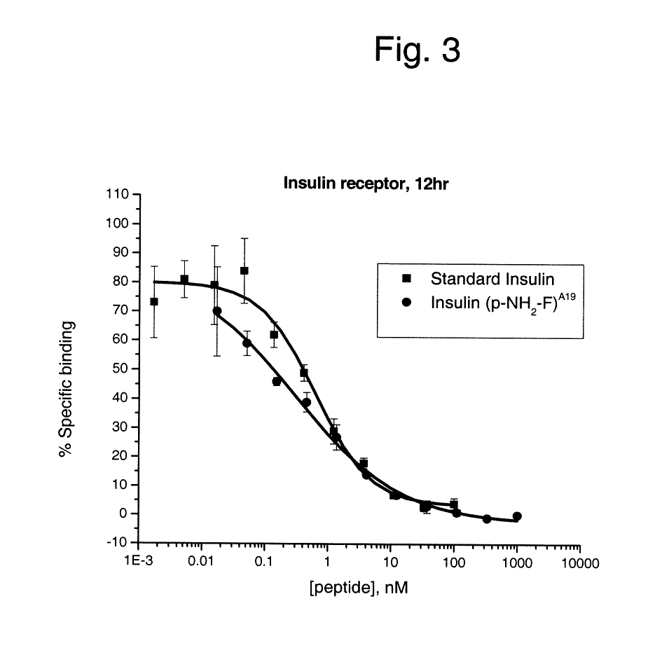

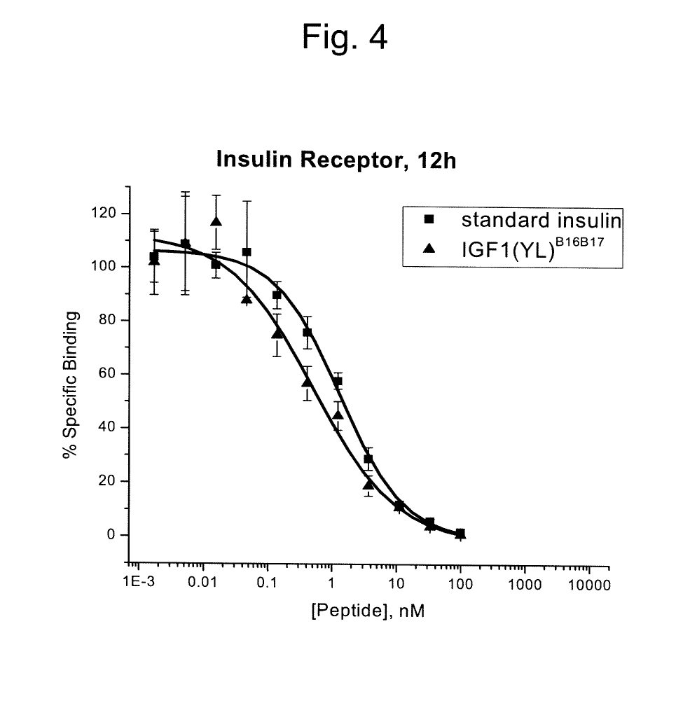

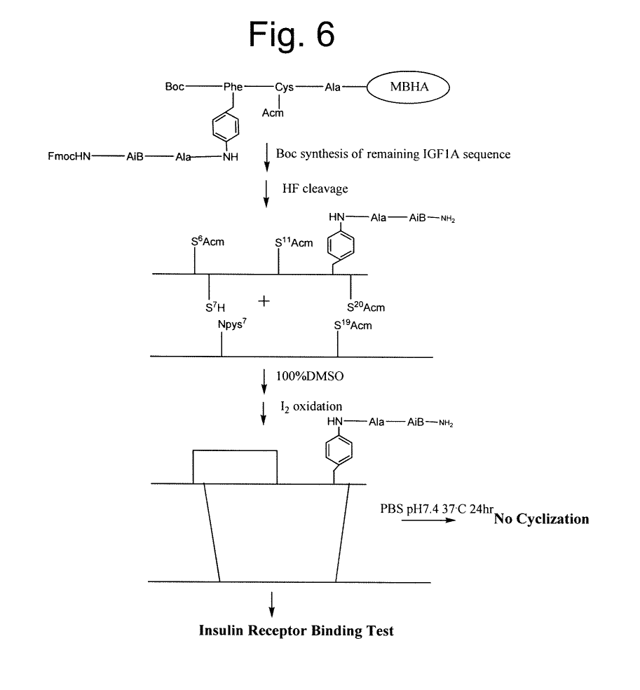

Applicants have discovered that the selective insertion of a 4-amino phenylalanine amino acid moiety for the native tyrosine at position 19 of the A chain can be accommodated without loss in potency of the insulin peptide (see FIG. 3). Subsequent chemical amidation of this active site amino group with the dipeptide prodrug moiety disclosed herein dramatically lessens insulin receptor binding activity and thus provides a suitable prodrug of insulin (see FIG. 6, data provided for the IGF1Y¹⁶L¹⁷ (p-NH₂—F)^{A19} analog which has been demonstrated to have comparable activity as insulin (p-NH₂—F)^{A19}, see FIG. 4). Applicants have discovered that a similar modification can be made to the IGF^{B16B17} derivative peptides to provide a suitable attachment site for prodrug chemistry. Accordingly, in one embodiment the dipeptide prodrug element is linked to the aromatic ring of an A19 4-aminophenylalanine of an IGF^{B16B17} derivative peptide via an amide bond, wherein the C-terminal amino acid of the dipeptide comprises an N-alkylated amino acid and the N-terminal amino acid of the dipeptide is any amino acid.

The dipeptide prodrug moiety can also be attached to additional sites of an IGF^{B16B17} derivative peptide to prepare IGF^{B16B17} derivative peptide prodrug analogs. In accordance with one embodiment an IGF^{B16B17} derivative peptide prodrug analog is provided comprising an IGF^{B16B17} derivative peptide A and B with a dipeptide prodrug element linked via an amide bond to the N-terminal amino group of the A chain or B chain, or the side chain amino group of an aromatic amine of a 4-amino-phenylalanine residue present at a position corresponding to A19, B16 or B25 of native insulin. In one embodiment the dipeptide comprises an N-terminal C-alkylated amino acid followed by an N-alkylated amino acid. The A chain and B chain comprising the IGF^{B16B17} derivative peptide prodrug analog may comprise the sequence of SEQ ID NO: 5 and SEQ ID NO: 11, respectively, or may comprise a derivative of SEQ ID NO: 5 and/or SEQ ID NO: 11 wherein the derivatives include substitution of the amino acid at position A19, B16 or B25 with a 4-amino phenylalanine and/or one or more amino acid substitutions at positions corresponding to positions A5, A8, A9, A10, A14, A15, A17, A18, A19 and A21, B1, B2, B3, B4, B5, B9, B10, B13, B14, B20, B22, B23, B26, B27, B28, B29 and B30 of native insulin, or deletions of any or all of corresponding positions B1-4 and B26-30, relative to native insulin. In one embodiment the dipeptide is linked to an N-terminal amino group of the A or B chain, wherein the C-terminal amino acid of the dipeptide comprises an N-alkylated amino acid and the N-terminal amino acid of the dipeptide is any amino acid, with the proviso that when the C-terminal amino acid of the dipeptide is proline, the N-terminal amino acid of the dipeptide comprises a C-alkylated amino acid.

In one embodiment the dipeptide prodrug element comprises the general structure of Formula I:

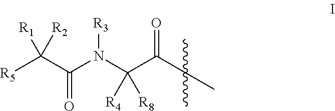

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH. In one embodiment when the prodrug element is linked to the N-terminal amine of the IGF^{B16B17} derivative peptide and R₄ and R₃ together with the atoms to which they are attached form a 4, 5 or 6 member heterocyclic ring, then at least one of R₁ and R₂ are other than H.

In one embodiment the prodrug element of Formula I is provided wherein R₁ is selected from the group consisting of H and C₁-C₈ alkyl; and R₂, R₈ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, and CH₂(C₅-C₉ heteroaryl), or R₁ and R₂ together with the atoms to which they are attached form a C₃-C₈ cycloalkyl ring;

R₃ is selected from the group consisting of C₁-C₈ alkyl, (C₁-C₄ alkyl)OH, (C₁-C₄ alkyl)SH, (C₁-C₄ alkyl)NH₂, (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring; and

R₇ is selected from the group consisting of H and OH and R₈ is H. In one embodiment R₃ is C₁-C₈ alkyl and R₄ is selected from the group consisting of H, C₁-C₆ alkyl, CH₂OH, (C₀-C₄ alkyl)(C₆-C₁₀ aryl)R₇, and CH₂(C₅-C₉ heteroaryl) or R₄ and R₃ together with the atoms to which they are attached form a 5 or 6 member heterocyclic ring. In a further embodiment R₅ is NHR₆ and R₈ is H.

In accordance with one embodiment the dipeptide element comprises a compound having the general structure of Formula I:

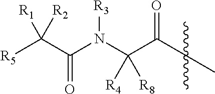

wherein

R_{1,} R_{2,} R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In another embodiment the dipeptide prodrug element comprises the general structure:

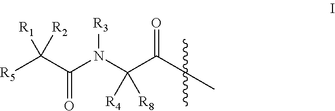

wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+)NH₂, (C₀-C₄ alkyl)(C₃-C₆ cycloalkyl), (C₀-C₄ alkyl)(C₂-0₅ heterocyclic), (C₀C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is selected from the group consisting of C₁-C₈ alkyl, (C₁-C₄ alkyl)OH, (C₁-C₄ alkyl)NH₂, (C₁-C₄ alkyl)SH, (C₃-C₆)cycloalkyl or R₄ and R₃ together with the atoms to which they are attached form a 5 or 6 member heterocyclic ring;

R₅ is NHR₆ or OH;

R₆ is H, C₁-C₈ alkyl, or R₆ and R₂ together with the atoms to which they are attached form a 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH and halo, provided that when R₄ and R₃ together with the atoms to which they are attached form a 5 or 6 member heterocyclic ring, both R₁ and R₂ are not H. In one embodiment either the first amino acid and/or the second amino acid of the dipeptide prodrug element is an amino acid in the D stereoisomer configuration.

In a further embodiment the prodrug element of Formula I is provided wherein

R₁ is selected from the group consisting of H and C₁-C₈ alkyl; and

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, and CH₂(C₅-C₉ heteroaryl), or R₁ and R₂ together with the atoms to which they are attached form a C₃-C₈ cycloalkyl ring;

R₃ is selected from the group consisting of C₁-C₈ alkyl, (C ₁-C₄ alkyl)OH, (C₁-C₄ alkyl)SH, (C₁-C₄ alkyl)NH₂, (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring;

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH and halo, and R₈ is H, provided that when the dipeptide element is linked to an N terminal amine and R₄ and R₃ together with the atoms to which they are attached form a 5 or 6 member heterocyclic ring, both R₁ and R₂ are not H. In one embodiment either the first amino acid and/or the second amino acid of the dipeptide prodrug element is an amino acid in the D stereoisomer configuration.

In other embodiments the dipeptide prodrug element has the structure of Formula I, wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is C₁-C₁₈ alkyl;

R₅ is NHR₆;

R₆ is H or C₁-C₈ alkyl; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In a further embodiment the dipeptide prodrug element has the structure of Formula I, wherein

R₁ and R₂ are independently C₁-C₁₈ alkyl or (C₀-C₄ alkyl)(C₆-C₁₀ aryl)R₇; or R₁ and R₂ are linked through —(CH₂)ₚ, wherein p is 2-9;

R₃ is C₁-C₁₈ alkyl;

R₄ and R₈ are each hydrogen;

R₅ is NH₂; and R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In a further embodiment the dipeptide prodrug element has the structure of Formula I, wherein

R₁ and R₂ are independently selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇, or R₁ and R₂ are linked through (CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and (C₀-C₄ alkyl)(C₆-C₁₀ aryl)R₇;

R₅ is NH₂; and

R₇ is selected from the group consisting of H, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl halo, with the proviso that both R₁ and R₂ are not hydrogen and provided that at least one of R₄ or R₈ is hydrogen.

In another embodiment the dipeptide prodrug element has the structure of Formula I, wherein

R₁ and R₂ are independently selected from the group consisting of hydrogen, C₁-C₈ alkyl and (C₁-C₄ alkyl)NH₂, or R₁ and R₂ are linked through (CH₂)ₚ, wherein p is 2-9;

R₃ is C₁-C₈ alkyl or R₃ and R₄ together with the atoms to which they are attached form a 4-6 heterocyclic ring;

R₄ is selected from the group consisting of hydrogen and C₁-C₈ alkyl;

R₈ is hydrogen; and

R₅ is NH₂, with the proviso that both R₁ and R₂ are not hydrogen.

In a further embodiment the dipeptide prodrug element has the structure of Formula I, wherein

R₁ and R₂ are independently selected from the group consisting of hydrogen, C₁-C₈ alkyl and (C₁-C₄ alkyl)NH₂;

R₃ is C₁-C₆ alkyl;

R₄ and R₈ are each hydrogen; and

R₅ is NH₂, with the proviso that both R₁ and R₂ are not hydrogen.

In another embodiment the dipeptide prodrug element has the structure of Formula I, wherein

R₁ and R₂ are independently selected from the group consisting of hydrogen and C₁-C₈ alkyl, (C₁-C₄ alkyl)NH₂, or R₁ and R₂ are linked through (CH₂)ₚ, wherein p is 2-9;

R₃ is C₁-C₈ alkyl;

R₄ is (C₀-C₄ alkyl)(C₆-C₁₀ aryl)R₇;

R₅ is NH₂;

R₇ is selected from the group consisting of hydrogen, C₁-C₈ alkyl and (C₀-C₄ alkyl)OH; and

R₈ is hydrogen, with the proviso that both R₁ and R₂ are not hydrogen.

In another embodiment the dipeptide prodrug element has the structure of Formula I, wherein

R₁ is selected from the group consisting of hydrogen, C₁-C₈ alkyl and (C₀-C₄ alkyl)(C₆-C₁₀ aryl)R₇;

R₂ is hydrogen;

R₃ is C₁-C₁₈ alkyl;

R₄ and R₈ are each hydrogen;

R₅ is NHR₆ or OH;

R₆ is H, C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo, with the proviso that, if R₁ is alkyl or (C₀-C₄ alkyl)(C₆-C₁₀ aryl)R₇, then R₁ and R₆ together with the atoms to which they are attached form a 4-11 heterocyclic ring. In one embodiment an insulin-like growth factor analog is provided comprising an A chain and a B chain wherein said A chain comprises a sequence of Z-GIVX₄ECCX₈X₉SCDLX₁₄X₁₅LEX₁₈X₁₉CX₂₁—R₁₃ (SEQ ID NO: 19) or a sequence that differs from SEQ ID NO: 19 by 1 to 3 amino acid modifications selected from positions 5, 8, 9, 10, 14, 15, 17, 18 and 21 of SEQ ID NO: 19, and said B chain sequence comprises a sequence of J-R₂₂—X₂₅LCGX₂₉X₃₀LVX₃₃X₃₄LX₃₆LVCGDX₄₂GFX₄₅ (SEQ ID NO: 20) or a sequence that differs from SEQ ID NO: 20 by 1 to 3 amino acid modifications selected from positions 5, 6, 9, 10, 16, 18, 19 and 21 of SEQ ID NO: 20;

wherein Z and J are independently H or a dipeptide element comprising the general structure of U—O, wherein U is an amino acid or a hydroxyl acid and O is an N-alkylated amino acid linked through an amide bond;

X₄ is aspartic acid or glutamic acid;

X₈ is histidine or phenylalanine;

X₉ and X₁₄ are independently selected from arginine, ornithine or alanine;

X₁₅ is arginine, ornithine or leucine;

X₁₈ is methionine, asparagine or threonine;

X₁₉ is an amino acid of the general structure:

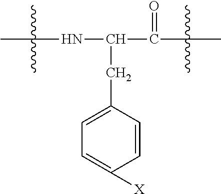

wherein X is selected from the group consisting of OH or NHR₁₀, wherein R₁₀ is H or a dipeptide element comprising the general structure U—O, wherein U is an amino acid or a hydroxyl acid and O is an N-alkylated amino acid;

X₂₁ is alanine, glycine or asparagine;

R₂₂ is selected from the group consisting of a covalent bond, AYRPSE (SEQ ID NO: 14), a glycine-proline-glutamic acid tripeptide, a proline-glutamic acid dipeptide and glutamic acid;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₃₆ is an amino acid of the general structure

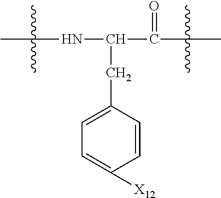

wherein X₁₂ is selected from the group consisting of OH and NHR₁₁, wherein R₁₁ is a dipeptide element comprising the general structure U—O;

X₄₂ is selected from the group consisting of alanine and arginine;

X₄₅ is an amino acid of the general structure

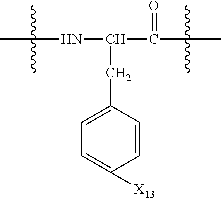

wherein X₁₃ is selected from the group consisting of OH and NHR₁₂, wherein R₁₂ is a dipeptide element comprising the general structure U—O; and

R₁₃ is COOH or CONH₂, with the proviso that one and only one of X, X₁₂, X₁₃, J and Z comprises U—O. In one embodiment J and Z are each H, X₁₂ and X₁₃ are each OH, and X is NH—U—O. In one embodiment U and O are selected to inhibit enzymatic cleavage of the U—O dipeptide from an insulin peptide by enzymes found in mammalian serum. In one embodiment U and/or O are selected such that the cleavage half-life of U—O from the insulin peptide, in PBS under physiological conditions, is not more than two fold the cleavage half-life of U—O from the insulin peptide in a solution comprising a DPP-IV protease (i.e., cleavage of U—O from the insulin prodrug does not occur at a rate more than 2× faster in the presence of DPP-IV protease and physiological conditions relative to identical conditions in the absence of the enzyme). In one embodiment U, O, or the amino acid of the insulin peptide to which U—O is linked is a non-coded amino acid. In one embodiment U and/or O is an amino acid in the D stereoisomer configuration. In some exemplary embodiments, U is an amino acid in the D stereoisomer configuration and O is an amino acid in the L stereoisomer configuration. In some exemplary embodiments, U is an amino acid in the L stereoisomer configuration and O is an amino acid in the D stereoisomer configuration. In some exemplary embodiments, U is an amino acid in the D stereoisomer configuration and O is an amino acid in the D stereoisomer configuration. In one embodiment U—O is a dipeptide comprising the structure of Formula I as defined herein. In one embodiment O is an N-alkylated amino acid but is not proline.

In accordance with one embodiment a prodrug form of IGF^{B16B17} derivative peptide is provided comprising an A chain comprising the sequence GIVX₄ECCX₈X₉SCDLRRLEMX₁₉CX₂₁—R₁₃ (SEQ ID NO: 19) and a B chain comprising the sequence X₂₅LCGAX₃₀LVDALYLVCGDX₄₂GFY (SEQ ID NO: 18), wherein

X₄ is aspartic acid or glutamic acid;

X₈ is phenylalanine or histidine;

X₉ is arginine, ornathine or alanine;

X₁₉ is an amino acid of the general structure

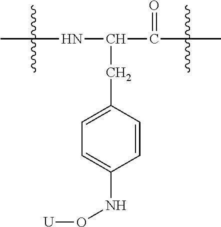

wherein U is an amino acid or a hydroxyl acid and O is an N-alkylated amino acid linked through an amide bond;

X₂₁ is alanine or asparagine;

X₂₅ is histidine or threonine;X₃₀ is selected from the group consisting of aspartic acid, glutamic acid, homocysteic acid and cysteic acid;

X₄₂ is selected from the group consisting of alanine ornathine and arginine; and R₁₃ is COOH or CONH₂. In one embodiment R₁₃ is COOH and the carboxy terminal amino acid of the B chain has an amide (CONH₂) in place of the natural alpha carbon carboxy group. In one embodiment X₄ is aspartic acid. In one embodiment the B chain comprises the sequence R₂₂—X₂₅LCGAX₃₀LVDALYLVCGDX₄₂GFY—R₄₇—R₄₈—R₄₉—R₁₄ (SEQ ID NO: 9), wherein

X₂₅ is histidine or threonine;

X₃₀ is glutamic acid;

X₄₂ is selected from the group consisting of alanine ornathine and arginine; R₂₂ is selected from the group consisting of the peptide of AYRPSE (SEQ ID NO: 14), PGPE (SEQ ID NO: 68), a glycine-proline-glutamic acid tripeptide, a proline-glutamic acid dipeptide, glutamic acid and an N-terminal amine, R₄₇ is a phenylalanine-asparagine dipeptide, a phenylalanine-serine dipeptide or a tyrosine-threonine dipeptide, R₄₈ is an aspartate-lysine dipeptide, an arginine-proline dipeptide, a proline-arginine dipeptide, a lysine-proline dipeptide, or a proline-lysine dipeptide, and R₄₉ is threonine or alanine; and R₁₃ and R₁₄ are independently COOH or CONH₂.

In accordance with one embodiment a prodrug form of an IGF^{B16B17} derivative peptide is provided comprising an A chain and a B chain wherein the A chain comprises a sequence of Z-GIVX₄ECCX₈X₉SCDLX₁₄X₁₅LEX₁₈X₁₉CX₂₁—R₁₃ (SEQ ID NO: 19) or a sequence that differs from SEQ ID NO: 19 by 1 to 3 amino acid modifications selected from positions 5, 8, 9, 10, 12, 14, 15, 17, 18 and 21 of SEQ ID NO: 19, and the B chain sequence comprises a sequence of J-R₂₂—X₂₅LCGX₂₉X₃₀LVX₃₃X₃₄LX₃₆LVCGDX₄₂GFX₄₅ (SEQ ID NO: 20) or a sequence that differs from SEQ ID NO: 20 by 1 to 3 amino acid modifications selected from positions 1, 2, 5, 6, 12, 13, 14, 15, 17, 18, 19, 20, and 21 of SEQ ID NO: 20 (corresponding to B5, B6, B9, B10, B16, B17, B18, B19, B21, B22, B23, B24 and B25 of native insulin);

wherein Z and J are independently H or a dipeptide comprising the general structure of Formula I:

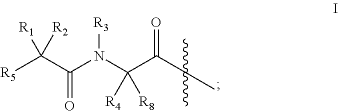

wherein

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH;

X₄ is aspartic acid or glutamic acid;

X₈ is histidine or phenylalanine;

X₉ and X₁₄ are independently selected from arginine, ornathine or alanine;

X₁₅ is arginine, ornathine, alanine or leucine;

X₁₈ is methionine, asparagine or threonine;

X₁₉ is an amino acid of the general structure

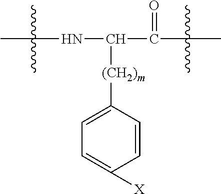

wherein X is selected from the group consisting of OH or NHR₁₀, wherein R₁₀ is a dipeptide comprising the general structure of Formula I:

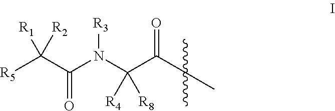

X₂₁ is alanine, glycine or asparagine;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ and X₄₁ are independently selected from the group consisting of aspartic acid and glutamic acid;

X₃₄ is selected from the group consisting of alanine and threonine;

X₃₆ is an amino acid of the general structure

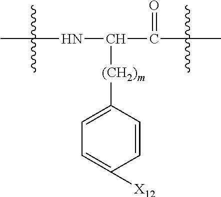

wherein X₁₂ is selected from the group consisting of OH and NHR₁₁, wherein R₁₁ is a dipeptide comprising the general structure of Formula I:

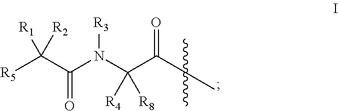

X₄₂ is arginine, ornathine or alanine;

X₄₅ is an amino acid of the general structure

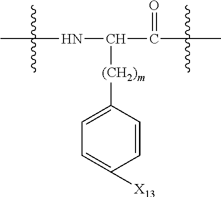

wherein X₁₃ is selected from the group consisting of OH and NHR₁₂, wherein R₁₂ is a dipeptide comprising the general structure of Formula I:

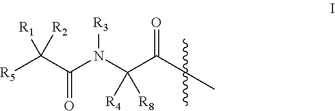

R₂₂ is a covalent bond or one to four amino acids;

R₁₃ is COOH or CONH₂; and

m is an integer selected from 0-3, with the proviso that one and only one of X, X₁₂, X₁₃, J and Z comprises a dipeptide of the general structure of Formula I:

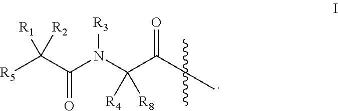

In one embodiment when J or Z comprise the dipeptide of Formula I, and R₄ and R₃ together with the atoms to which they are attached form a 4, 5 or 6 member heterocyclic ring, then both R₁ and R₂ are not hydrogen. In one embodiment R₁₃ is COOH and the carboxy terminal amino acid of the B peptide has an amide (CONH₂) in place of the natural alpha carbon carboxy group. In one embodiment R₂₂ is selected from the group consisting of a bond, the tripeptide glycine-proline-glutamic acid, the dipeptide proline-glutamic acid, and glutamic acid. In one embodiment m is 1. In one embodiment, m is 1 and the B chain comprises the sequence J-R₂₂—X₂₅LCGX₂₉X₃₀LVX₃₃X₃₄LX₃₆LVCGDX₄₂GFX₄₅—R₄₇—R₄₈—R₄₉—R₁₄ (SEQ ID NO: 20), wherein

X₂₅ is histidine or threonine;

X₂₉ is alanine or glycine;

X₃₀ is selected from the group consisting of aspartic acid, glutamic acid, homocysteic acid and cysteic acid;

X₃₃ is selected from the group consisting of aspartic acid and glutamic acid;

X₃₄ is selected from the group consisting of alanine and threonine;

X₃₆ is selected from the group consisting of phenylalanine and 4-amino-phenylalanine;

X₄₂ is selected from the group consisting of alanine, ornithine and arginine;

X₄₅ is selected from the group consisting of phenylalanine and 4-amino-phenylalanine;

R₁₃ is COOH and R₁₄ is CONH₂;

R₂₂ is selected from the group consisting of a covalent bond, AYRPSE (SEQ ID NO: 14), a glycine-proline-glutamic acid tripeptide, a proline-glutamic acid dipeptide, and glutamic acid; R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a lysine-proline dipeptide, or a proline-lysine dipeptide;

R₄₉ is threonine or alanine and R₁₄ is COOH or CONH₂. In a further embodiment, X, X₁₂ and X₁₃ are each OH, R₁₃ is COOH and R₁₄ is CONH₂ further provided that when R₄ and R₃ together with the atoms to which they are attached form a 5 or 6 member heterocyclic ring, then at least one of R₁ and R₂ are other than H.

In one embodiment an insulin-like growth factor analog is provided comprising an A chain and a B chain wherein said A chain comprises a sequence of GIVX₄ECCX₈X₉SCDLX₁₄X₁₅LEX₁₈X₁₉CX₂₁-R₁₃ (SEQ ID NO: 19) or a sequence that differs from SEQ ID NO: 19 by 1 to 3 amino acid modifications selected from positions 5, 8, 9, 10, 14, 15, 17, 18 and 21 of SEQ ID NO: 19, and said B chain sequence comprises a sequence of

R₂₂-X₂₅LCGX₂₉X₃₀LVX₃₃X₃₄LX₃₆LVCGDX₄₂GFX₄₅ (SEQ ID NO: 20) or a sequence that differs from SEQ ID NO: 20 by 1 to 3 amino acid modifications selected from positions 5, 6, 9, 10, 16, 18, 19 and 21 of SEQ ID NO: 20;

wherein

X₄ is aspartic acid or glutamic acid;

X₈ is histidine or phenylalanine;

X₉ and X₁₄ are independently selected from arginine, ornithine or alanine;

X₁₅ is arginine, ornithine or leucine;

X₁₈ is methionine, asparagine or threonine;

X₁₉ is an amino acid of the general structure:

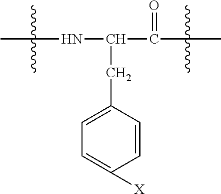

wherein X is selected from the group consisting of OH or NHR₁₀, wherein R₁₀ is a dipeptide element comprising the general structure U—O, wherein U is an amino acid or a hydroxyl acid and O is an N-alkylated amino acid;

X₂₁ is alanine, glycine or asparagine;

R₂₂ is selected from the group consisting of a covalent bond, AYRPSE (SEQ ID NO: 14), a glycine-proline-glutamic acid tripeptide, a proline-glutamic acid dipeptide and glutamic acid;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₃₆ is tyrosine;

X₄₂ is selected from the group consisting of alanine and arginine;

X₄₅ is tyrosine and phenylalanine; further wherein the B chain comprises a carboxy terminal extension of 1 to 4 amino acids wherein said carboxy terminal extension comprises an amino acid having the structure of

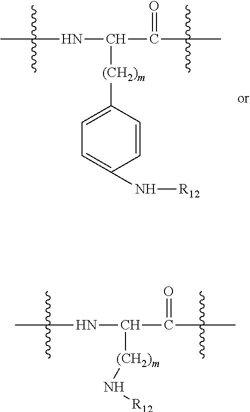

wherein m is an integer from 0-3;

n is an integer from 1-4;

R₁₂ is a dipeptide comprising the general structure U—O; and R₁₃ is COOH or CONH₂. In one embodiment U—O comprises the general structure of:

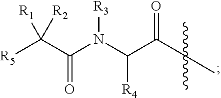

wherein R₁ is selected from the group consisting of H and C₁-C₈ alkyl; and

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, and CH₂(C₅-C₉ heteroaryl);

R₃ is selected from the group consisting of C₁-C₈ alkyl, (C ₁-C₄ alkyl)OH, (C₁-C₄ alkyl)SH, (C₁-C₄ alkyl)NH₂, (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring;

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo. In a further embodiment the A chain comprises the sequence GIVX₄ECCX₈X₉SCDLX₁₄X₁₅LEX₁₈X₁₉CX₂₁—R₁₃ (SEQ ID NO: 19) and the B chain comprises the sequence X₂₅LCGX₂₉X₃₀LVX₃₃X₃₄LYLVCGDX₄₂GFY (SEQ ID NO: 9), with the designations defined as immediately above.

In accordance with one embodiment a prodrug derivative of an IGF^{B16B17} derivative peptide is provided comprising an A chain comprising the sequence Z-GIVX₄X₅CCX₈X₉X₁₀CX₁₂LX₁₄X₁₅LEX₁₈X₁₉CX₂₁—R₁₃ (SEQ ID NO: 82) and a B chain having the sequence J-R₂₂—X₂₅LCGX₂₉X₃₀LVX₃₃X₃₄LYLVCGX₄₁X₄₂GFX₄₅R₄₇—R₄₈—R₄₉—R₁₄ (SEQ ID NO: 67), wherein

Z and J are independently H or a dipeptide comprising the general structure of Formula I:

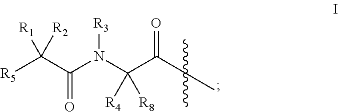

wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃,(C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is selected from the group consisting of C₁-C₈ alkyl, (C₁-C₄ alkyl)OH, (C₁-C₄ alkyl)NH₂, (C₁-C₄ alkyl)SH, (C₃-C₆)cycloalkyl or R₄ and R₃ together with the atoms to which they are attached form a 5 or 6 member heterocyclic ring;

R₅ is NHR₆ or OH;

R₆ is H, C₁-C₈ alkyl, or R₆ and R₂ together with the atoms to which they are attached form a 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH and halo, provided that when R₄ and R₃ together with the atoms to which they are attached form a 5 or 6 member heterocyclic ring, both R₁ and R₂ are not H;

X₄ is glutamic acid or aspartic acid;

X₅ is glutamic acid or glutamine;

X₈ is histidine, threonine or phenylalanine;

X₉ is serine, ornathine, arginine or alanine;

X₁₀ is serine or isoleucine;

X₁₂ is serine or aspartic acid;

X₁₄ are independently selected from tyrosine, ornathine, arginine or alanine;

X₁₅ is glutamine, ornathine, arginine, alanine or leucine;

X₁₈ is methionine, asparagine or threonine;

X₁₉ is an amino acid of the general structure

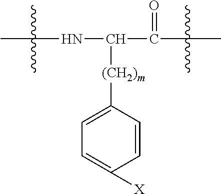

wherein X is selected from the group consisting of OH or NHR₁₀, wherein R₁₀ is a dipeptide comprising the general structure of Formula I:

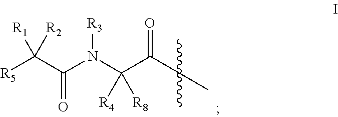

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid and aspartic acid;

X₄₂ is selected from the group consisting of alanine, ornithine and arginine;

X₄₅ is an amino acid of the general structure

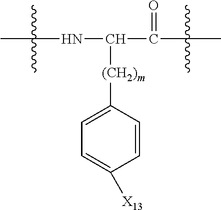

wherein X₁₃ is selected from the group consisting of OH and NHR₁₂, wherein R₁₂ is a dipeptide comprising the general structure of Formula I:

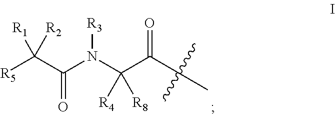

R₁₃ and R₁₄ are independently COOH or CONH₂;

R₂₂ is selected from the group consisting of a bond, the tripeptide glycine-proline-glutamic acid, the dipeptide proline-glutamic acid, and glutamic acid;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a proline-arginine dipeptide, a lysine-proline dipeptide, or a proline-lysine dipeptide;

R₄₉ is threonine or alanine; and R₁₃ and R₁₄ are independently selected from COOH and CONH₂,

m is an integer selected from 0-3, with the proviso that the B chain is not a native insulin B chain sequence (e.g., not SEQ ID NO: 2) and that one and only one of X, X₁₃, J and Z comprises a dipeptide of the general structure of Formula I:

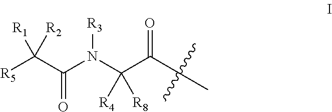

and when J or Z comprise the dipeptide of Formula I, and R₄ and R₃ together with the atoms to which they are attached form a 4, 5 or 6 member heterocyclic ring, then both R₁ and R₂ are not hydrogen.

In accordance with one embodiment a prodrug form of a IGF^{B16B17} derivative peptide is provided comprising an A chain having the sequence GIVX₄X₅CCX₈X₉X₁₀CX₁₂LX₁₄X₁₅LEX₁₈X₁₉CX₂₁—R₁₃ (SEQ ID NO: 82) or a peptide that differs from SEQ ID NO: 82 by one or two conservative amino acid substitutions and a B chain having the sequence R₂₂—X₂₅LCGX₂₉X₃₀LVX₃₃X₃₄LYLVCGDX₄₂GFX₄₅R₄₇—R₄₈—R₄₉—R₁₄ (SEQ ID NO: 67) or a peptide that differs from SEQ ID NO: 67 by one or two conservative amino acid substitutions, wherein

X₄ is glutamic acid or aspartic acid;

X₅ is glutamic acid or glutamine;

X₈ is histidine, threonine or phenylalanine;

X₉ is serine, arginine or alanine;

X₁₀ is serine or isoleucine;

X₁₂ is serine or aspartic acid;

X₁₄ are independently selected from tyrosine, arginine or alanine;

X₁₅ is glutamine, arginine, alanine or leucine;

X₁₈ is methionine, asparagine or threonine;

X₁₉ is an amino acid of the general structure

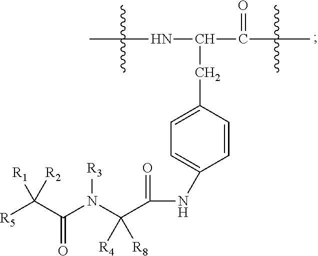

wherein

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-0₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of ornathine and arginine;

X₄₅ is phenylalanine or tyrosine;

R₁₃ and R₁₄ are independently COOH or CONH₂;

R₂₂ is selected from the group consisting of the tripeptide glycine-proline-glutamic acid, the dipeptide proline-glutamic acid, glutamic acid and an N-terminal amine;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a proline-arginine dipeptide, a lysine-proline dipeptide, or a proline-lysine dipeptide;

R₄₉ is threonine or alanine; and R₁₃ and R₁₄ are independently selected from COOH and CONH₂, with the proviso that the B chain is not a native insulin B chain sequence (e.g., not SEQ ID NO: 2).

In accordance with one embodiment a prodrug form of a IGF^{B16B17} derivative peptide is provided comprising an A chain comprising the sequence GIVX₄ECCX₈X₉SCDLX₁₄X₁₅LEX₁₈X₁₉CX₂₁—R₁₃ (SEQ ID NO: 19) and a B chain comprising the sequence X₂₅LCGX₂₉X₃₀LVX₃₃X₃₄LYLVCGDX₄₂GFY (SEQ ID NO: 9), wherein

X₄ is aspartic acid or glutamic acid;

X₈ is phenylalanine or histidine;

X₉ is arginine, ornathine or alanine;

X₁₄ is arginine or alanine;

X₁₅ is arginine or leucine;

X₁₈ is methionine or threonine;

X₁₉ is an amino acid of the general structure

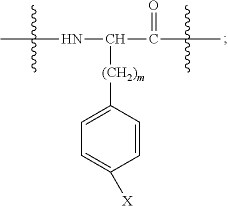

wherein X is selected from the group consisting of OH or NHR₁₀, wherein R₁₀ is a dipeptide comprising the general structure of Formula I:

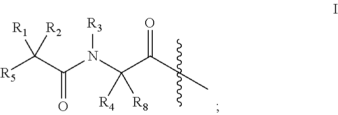

X₂₁ is alanine, glycine or asparagine;

X₂₅ is histidine or threonine;

X₂₉ is selected from the group consisting of alanine and glycine;

X₃₀ is selected from the group consisting of aspartic acid, glutamic acid, homocysteic acid and cysteic acid;

X₃₃ is aspartic acid;

X₃₄ is selected from the group consisting of alanine and threonine; and

X₄₂ is selected from the group consisting of alanine ornathine and arginine; and R₁₃ is COOH or CONH₂.

In one embodiment a prodrug form of IGF^{B16B17} derivative peptide is provided comprising an A chain comprising the sequence GIVDECCX₈X₉SCDLRRLEMX₁₉CX₂₁—R₁₃ (SEQ ID NO: 21) and a B chain comprising the sequence X₂₅LCGAX₃₀LVDALYLVCGDX₄₂GFY (SEQ ID NO: 18), wherein

X₈ is phenylalanine or histidine;

X₉ is arginine, ornathine or alanine;

X₁₉ is an amino acid of the general structure

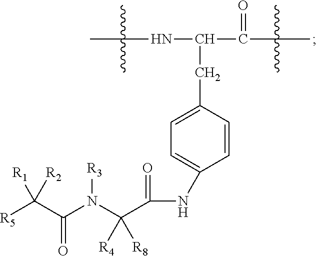

wherein

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

X₂₁ is alanine or asparagine;

X₂₅ is histidine or threonine;

X₃₀ is selected from the group consisting of aspartic acid, glutamic acid, homocysteic acid and cysteic acid;

X₄₂ is selected from the group consisting of alanine, ornathine and arginine; and R₁₃ is COOH or CONH₂. In one embodiment R₁₃ is COOH and the carboxy terminal amino acid of the B peptide has an amide (CONH₂) in place of the natural alpha carbon carboxy group. In one embodiment X₃₀ is glutamic acid and X₄₂ is arginine. In one embodiment the B chain comprises the sequence R₂₂—X₂₅LCGAX₃₀LVDALYLVCGDX₄₂GFY—R₄₇—R₄₈—R₄₉—R₁₄ (SEQ ID NO: 9), wherein R₂₂ is selected from the group consisting of the peptide of AYRPSE (SEQ ID NO: 14), a glycine-proline-glutamic acid tripeptide, a proline-glutamic acid dipeptide, glutamic acid and an N-terminal amine, X₃₀ is glutamic acid, X₄₂ is arginine, R₄₇ is a phenylalanine-asparagine dipeptide or a phenylalanine-serine dipeptide, R₄₈ is an aspartate-lysine dipeptide, an arginine-proline dipeptide, a proline-arginine dipeptide, a lysine-proline dipeptide, or a proline-lysine dipeptide, and R₄₉ is threonine or alanine; and R₁₃ and R₁₄ are independently COOH or CONH₂.

In a further embodiment a prodrug form of IGF^{B16B17} derivative peptide comprises an A chain having the sequence GIVDECCX₈X₉SCDLX₁₄X₁₅LEX₁₈X₁₉CX₂₁—R₁₃ (SEQ ID NO: 13) and a B chain having the sequence of R₂₂—X₂₅LCGX₂₉X₃₀LVX₃₃X₃₄LYLVCGDX₄₂GFY—R₄₇—R₄₈—R₄₉—R₁₄ (SEQ ID NO: 9) wherein

X₈ is histidine or phenylalanine;

X₉ and X₁₄ are independently selected from arginine, ornathine or alanine;

X₁₅ is arginine, ornathine or leucine;

X₁₈ is methionine, asparagine or threonine;

X₁₉ is an amino acid of the general structure

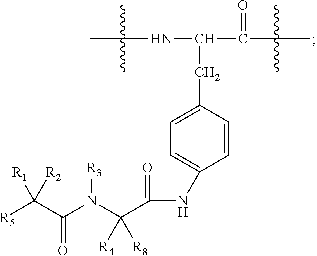

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-0₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

X₂₁ is alanine, glycine or asparagine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of alanine, ornathine and arginine;

R₁₃ and R₁₄ are independently COOH or CONH₂;

R₂₂ is selected from the group consisting of AYRPSE (SEQ ID NO: 14), PGPE (SEQ ID NO: 68), a glycine-proline-glutamic acid tripeptide, a proline-glutamic acid dipeptide, glutamic acid and an N-terminal amine;

R₄₇ is a phenylalanine-asparagine dipeptide, a phenylalanine-serine dipeptide or a tyrosine-threonine dipeptide;

R₄₈ is an aspartate-lysine dipeptide, an arginine-proline dipeptide, a lysine-proline dipeptide, or a proline-lysine dipeptide; and

R₄₉ is threonine or alanine; and R₁₃ and R₁₄ are independently COOH or CONH₂ and R₁₃ and R₁₄ are independently COOH or CONH₂.

In one embodiment a prodrug derivative of an IGF^{B16B17} derivative peptide having high specificity for the insulin receptor is provided, wherein the peptide comprises an A chain having the sequence GIVDECCX₈X₉SCDLRRLEMX₁₉CX₂₁—R₁₃ (SEQ ID NO: 69) and a B chain comprising the sequence R₂₂—X₂₅LCGAX₃₀LVDALYLVCGDX₄₂GFY (SEQ ID NO: 18), wherein

X₈ is histidine or phenylalanine;

X₉ is arginine or alanine;

X₁₉ is an amino acid of the general structure

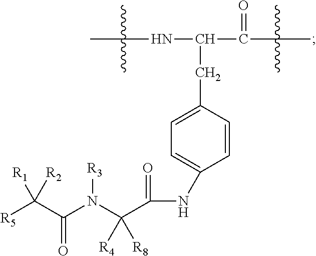

wherein

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

X₂₁ is alanine, glycine or asparagine;

R₂₂ is selected from the group consisting of a glycine-proline-glutamic acid tripeptide, a proline-glutamic acid dipeptide, glutamic acid and an N-terminal amine;

X₂₅ is histidine or threonine;

X₃₀ is selected from the group consisting of aspartic acid and glutamic acid;

X₄₂ is arginine, alanine or ornathine;

R₁₃ is COOH and the carboxy terminal amino acid of the B chain has an amide (CONH₂) in place of the native alpha carbon carboxylic acid. In one embodiment a prodrug derivative of an IGF^{B16B17} derivative peptide having high specificity for the insulin receptor is provided wherein the peptide comprises an A chain having the sequence GIVDECCFRSCDLRRLEMX₁₉CA-R₁₃ (SEQ ID NO: 22) and a B chain having the sequence R₂₂-TLCGAELVDALYLVCGDRGFYFNKPT-R₁₄ (SEQ ID NO: 64), wherein

X₁₉ is an amino acid of the general structure

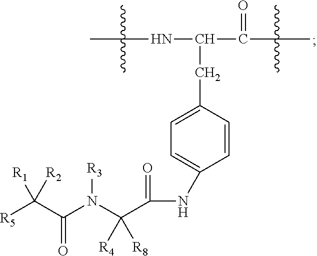

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

R₂₂ is selected from the group consisting of a glycine-proline-glutamic acid tripeptide, a proline-glutamic acid dipeptide, glutamic acid and an N-terminal amine; and

R₁₃ and R₁₄ are independently COOH or CONH₂. In one embodiment an IGF^{B16B17} derivative peptide having high specificity for the insulin receptor is provided wherein the peptide comprises an A chain comprising the sequence GIVDECCFRSCDLRRLEMX₁₉CA-R₁₃ (SEQ ID NO: 70) and a B chain comprising the sequence GPETLCGAELVDALYLVCGDRGFYFNKPT-R₁₄ (SEQ ID NO: 11) or AYRPSETLCGGELVDTLYLVCGDRGFYFSRPA-R₁₄ (SEQ ID NO: 12), wherein X₁₉ is an amino acid of the general structure

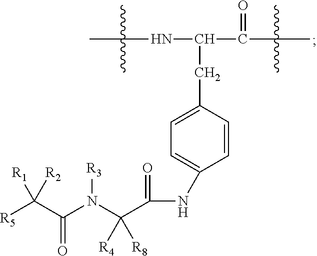

wherein

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo; and

R₁₃ and R₁₄ are independently COOH or CONH₂.

In another embodiment a prodrug derivative of an IGF^{B16B17} derivative peptide having high specificity for the insulin receptor is provided wherein the peptide comprises an A chain comprising the sequence GIVDECCX₈X₉SCDLRRLEMX₁₉CA-R₁₃ (SEQ ID NO: 21) and a B chain comprising the sequence GPETLCGAELVDALYLVCGDRGFY—R₁₄ (SEQ ID NO: 11), wherein

X₈ is histidine or phenylalanine;

X₉ is arginine or alanine;

X₁₉ is an amino acid of the general structure

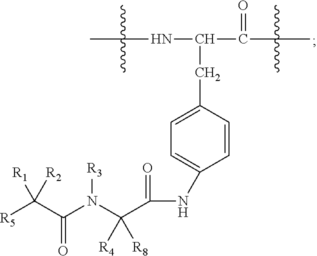

wherein

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo; and

R₁₃ and R₁₄ are independently COOH or CONH₂.

The IGF^{B16B17} derivative peptide prodrugs disclosed herein may be part of a dimer, trimer or higher order multimer comprising at least two, three, or more peptides bound via a linker, wherein at least one or both peptides is an IGF^{B16B17} derivative peptide. The dimer comprises either two single chain insulin/IGF^{B16B17} derivative peptides, or two A chain/B chain heterodimers or a combination thereof. The dimer may be a homodimer or heterodimer, comprising peptides selected from the group consisting of native insulin, native IGF-1, native IGF-II, an insulin analog peptide, and IGF^{B16B17} derivative peptides (as either single chain peptides or as heterodimers of the A and B chains). In some embodiments, the linker is selected from the group consisting of a bifunctional thiol crosslinker and a bi-functional amine crosslinker. In certain embodiments, the linker is PEG, e.g., a 5 kDa PEG, 20 kDa PEG. In some embodiments, the linker is a disulfide bond.

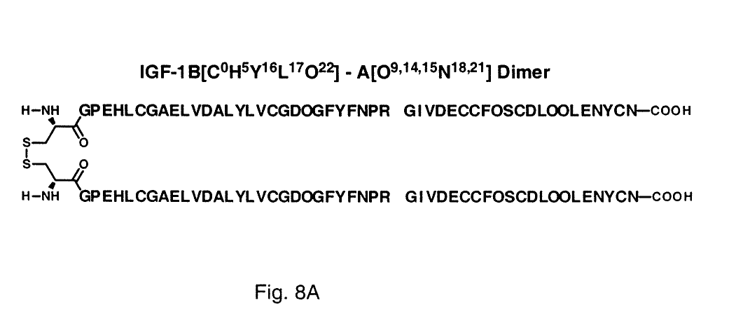

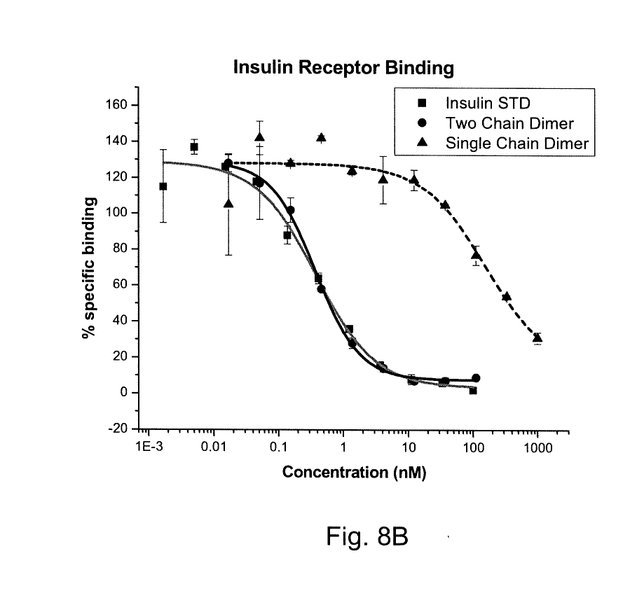

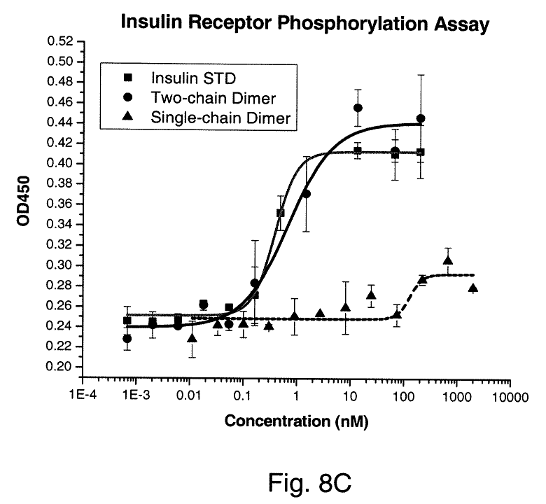

For example, each monomer of the dimer may comprise a Cys residue (e.g., a terminal or internally positioned Cys) and the sulfur atom of each Cys residue participates in the formation of the disulfide bond. In some aspects of the invention, the monomers are connected via terminal amino acids (e.g., N-terminal or C-terminal; see FIG. 8A), via internal amino acids, or via a terminal amino acid of at least one monomer and an internal amino acid of at least one other monomer. In specific aspects, the monomers are not connected via an N-terminal amino acid. In some aspects, the monomers of the multimer are attached together in a “tail-to-tail” orientation in which the C-terminal amino acids of each monomer are attached together. A conjugate moiety may be covalently linked to any of the IGF^{B16B17} derivative peptides described herein, including a dimer, trimer or higher order multimer.

In accordance with one embodiment the dipeptide of Formula I is further modified to comprise a large polymer that interferes with the IGF^{B16B17} derivative peptide's ability to interact with the insulin or IGF-1 receptor. Subsequent cleavage of the dipeptide releases the IGF^{B16B17} derivative peptide from the dipeptide complex wherein the released IGF^{B16B17} derivative peptide is fully active. In accordance with one embodiment the dipeptide of Formula I is further modified to comprises a large polymer that interferes with the bound IGF^{B16B17} derivative peptide's ability to interact with the insulin or IGF-1 receptor. In accordance with one embodiment one of X, X₁₂, X₁₃, J and Z comprises a dipeptide of the general structure of Formula I:

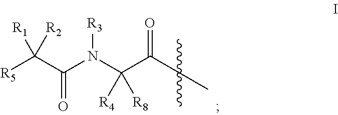

wherein the dipeptide of Formula I is pegylated or acylated. In one embodiment either J, Z or X comprises an acylated or pegylated dipeptide of Formula I, and in one embodiment J comprises an acylated or pegylated dipeptide of Formula I.

In accordance with one embodiment the dipeptide of Formula I further comprises an polyethylene oxide, alkyl or acyl group. In one embodiment one or more polyethylene oxide chains are linked to the dipeptide of Formula I wherein the combined molecular weight of the polyethylene oxide chains ranges from about 20,000 to about 80,000 Daltons, or 40,000 to 80,000 Daltons or 40,000 to 60,000 Daltons. In one embodiment the polyethylene oxide is polyethylene glycol. In one embodiment at least one polyethylene glycol chain having a molecular weight of about 40,000 Daltons is linked to the dipeptide of Formula I. In another embodiment the dipeptide of Formula I is acylated with an acyl group of sufficient size to bind serum albumin and thus inactivate the IGF^{B16B17} derivative peptide upon administration. The acyl group can be linear or branched, and in one embodiment is a C16 to C30 fatty acid. For example, the acyl group can be any of a C16 fatty acid, C18 fatty acid, C20 fatty acid, C22 fatty acid, C24 fatty acid, C26 fatty acid, C28 fatty acid, or a C30 fatty acid. In some embodiments, the acyl group is a C16 to C20 fatty acid, e.g., a C18 fatty acid or a C20 fatty acid.

In accordance with one embodiment a prodrug form of an IGF^{B16B17} derivative peptide is provided comprising an A chain having the sequence Z-GIVDECCX₈X₉SCDLRRLEMX₁₉CX₂₁—R₁₃ (SEQ ID NO: 21) and a B chain having the sequence J-R₂₂—X₂₅LCGAX₃₀LVDALYLVCGDX₄₂GFYFN—R₄₈—R₄₉—R₁₄ (SEQ ID NO: 15), wherein

wherein Z and J are independently H or a dipeptide comprising the general structure:

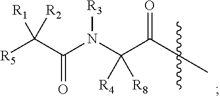

X₈ is histidine or phenylalanine;

X₉ is arginine or alanine;

X₁₉ is an amino acid of the general structure

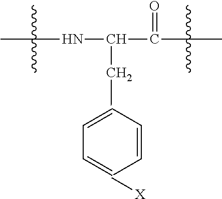

wherein X is selected from the group consisting of OH or NHR₁₀, wherein R₁₀ is a dipeptide comprising the general structure:

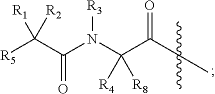

X₂₁ is alanine, glycine or asparagine;

X₂₅ is histidine or threonine;

X₃₀ is selected from the group consisting of histidine, aspartic acid, glutamic acid, homocysteic acid and cysteic acid;

X₄₂ is selected from the group consisting of alanine and arginine;

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH;

R₁₃ is COOH and R₁₄ is CONH₂;

R₂₂ is selected from the group consisting of a covalent bond, AYRPSE (SEQ ID NO: 14), a glycine-proline-glutamic acid tripeptide, a proline-glutamic acid dipeptide, and glutamic acid;

R₄₈ is an aspartate-lysine dipeptide, an arginine-proline dipeptide, a lysine-proline dipeptide, or a proline-lysine dipeptide;

R₄₉ is threonine, with the proviso that one and only one of X, J and Z comprises a dipeptide of the general structure:

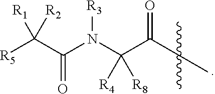

In one embodiment, when X is OH and R₄ and R₃ together with the atoms to which they are attached form a 5 or 6 member heterocyclic ring, at least one of R₁ and R₂ are other than H. In one embodiment Z and J are both H and X is NHR₁₀.

In a further embodiment, a prodrug derivative of an IGF/insulin co-agonist prodrug is provided comprising an A chain having the sequence Z-GIVDECCX₈X₉SCDLRRLEMX₁₉CX₂₁—R₁₃ (SEQ ID NO: 21) and a B chain having the sequence J-R₂₂—X₂₅LCGAX₃₀LVDALYLVCGDRGFYFNKPT-R₁₄ (SEQ ID NO: 17), wherein

Z and J are independently H or a dipeptide comprising the general structure:

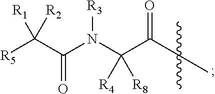

X₈ is histidine or phenylalanine;

X₉ is arginine or alanine;

X₉ is arginine or alanine;

X₁₉ is an amino acid of the general structure

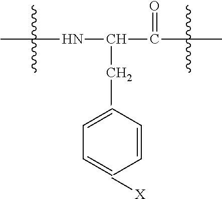

wherein X is selected from the group consisting of OH or NHR₁₀, wherein R₁₀ is a dipeptide comprising the general structure:

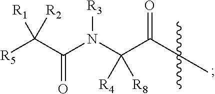

R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH;

R₁₃ is COOH and R₁₄ is CONH₂;

X₂₁ is alanine, glycine or asparagine;

X₂₅ is histidine or threonine;

X₃₀ is selected from the group consisting of aspartic acid and glutamic acid;

R₁₃ is COOH and R₁₄ is CONH₂; and

R₂₂ is selected from the group consisting of a covalent bond, the tripeptide glycine-proline-glutamic acid, the dipeptide proline-glutamic acid, and glutamic acid. In one embodiment, when X is OH and R₄ and R₃ together with the atoms to which they are attached form a 5 or 6 member heterocyclic ring, then both R₁ and R₂ are both other than H, with the proviso that one and only one of X, J and Z comprises a dipeptide of the general structure:

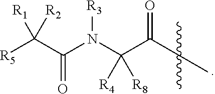

In one embodiment, when J or Z comprise the dipeptide of Formula I, and R₄ and R₃ together with the atoms to which they are attached form a 4, 5 or 6 member heterocyclic ring, then both R₁ and R₂ are not hydrogen. In one embodiment Z and J are both H and X is NHR₁₀.

In one embodiment a prodrug derivative of an IGF^{B16B17} derivative peptide having high specificity for the insulin receptor relative to the IGF I receptor is provided wherein the peptide comprises an A chain having the sequence GIVDECCX₈X₉SCDLRRLEMX₁₉CX₂₁—R₁₃ (SEQ ID NO: 21) and a B chain having the sequence R₂₂—X₂₅LCGAX₃₀LVDALYLVCGDX₄₂GFY (SEQ ID NO: 18), wherein

X₈ is histidine or phenylalanine;

X₉ is arginine or alanine;

X₁₉ is an amino acid of the general structure

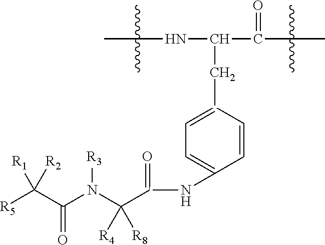

wherein R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl, and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH;

R₁₃ is COOH and the carboxy terminal amino acid of the B chain has an amide (CONH₂) in place of the native alpha carbon carboxylic acid;

X₂₁ is alanine, glycine or asparagine;

X₂₅ is histidine or threonine;

X₃₀ is selected from the group consisting of aspartic acid and glutamic acid;

X₄₂ is selected from the group consisting of alanine, arginine and ornathine;

R₂₂ is selected from the group consisting of a glycine-proline-glutamic acid tripeptide, a proline-glutamic acid dipeptide, glutamic acid and an N-terminal amine.

In one embodiment, an IGF^{B16B17} derivative peptide prodrug analog is provided comprising an A chain sequence of GIVDECCFRSCDLRRLEMX₁₉CA-R₁₃ (SEQ ID NO: 22) and a B chain sequence of R₂₂-TLCGAELVDALX₃₆LVCGDRGFX₄₅FNKPT-R₁₄ (SEQ ID NO: 23), or alternatively an A chain comprises the sequence of GIVDECCHASCDLRRLEMX₁₉CN—R₁₃ (SEQ ID NO: 24) and a B chain sequence of R₂₂—HLCGADLVDALX₃₆LVCGDAGFX₄₅FNKPT-R₁₄ (SEQ ID NO: 25), wherein

X₁₉ is an amino acid of the general structure

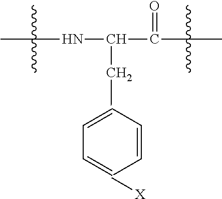

wherein X is selected from the group consisting of OH or NHR₁₀, wherein R₁₀ is a dipeptide comprising the general structure:

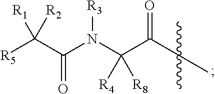

wherein R₁ is selected from the group consisting of H and C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, and CH₂(C₅-C₉ heteroaryl), or R₁ and R₂ together with the atoms to which they are attached form a C₃-C₆ cycloalkyl;

R₃ is selected from the group consisting of C₁-C₈ alkyl, (C₁-C₄ alkyl)OH, (C₁-C₄ alkyl)NH₂, (C₁-C₄ alkyl)SH, and (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring;

R₇ is selected from the group consisting of H and OH; and

R₈ is H;

X₃₆ is an amino acid of the general structure

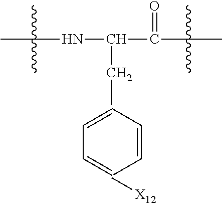

wherein X₁₂ is selected from the group consisting of OH and NHR₁₁, wherein R₁₁ is a dipeptide comprising the general structure:

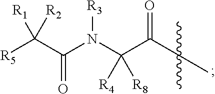

X₄₅ is an amino acid of the general structure

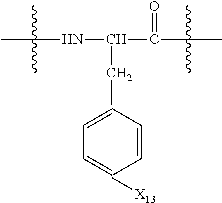

wherein X₁₃ is selected from the group consisting of OH and NHR₁₂, wherein R₁₂ is a dipeptide comprising the general structure:

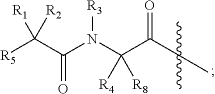

R₁₃ and R₁₄ are independently COOH or CONH₂;

R₂₂ is selected from the group consisting of a covalent bond, the tripeptide glycine-proline-glutamic acid, the dipeptide proline-glutamic acid, glutamic acid and an N-terminal amine, with the proviso that one and only one of X, X₁₂ and X₁₃, comprises a dipeptide of the general structure:

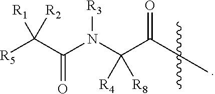

In one embodiment X₁₂ and X₁₃ are each OH and X is NHR₁₀. In a further embodiment X₁₂ and X₁₃ are each OH, X is NHR₁₀ and R₁₀ is COOH and R₁₄ is CONH₂.

In one embodiment, an IGF^{B16B17} derivative peptide prodrug analog is provided comprising an A chain sequence of GIVDECCFRSCDLRRLEMX₁₉CA-R₁₃ (SEQ ID NO: 22) and a B chain sequence of FVNQTLCGAELVDALYLVCGDRGFYFNKPX₄₉—R₁₄ (SEQ ID NO: 71), GPETLCGAELVDALYLVCGDRGFYFNKPT-R₁₄ (SEQ ID NO: 11) or AYRPSETLCGGELVDTLYLVCGDRGFYFSRPA-R₁₄ (SEQ ID NO: 12) wherein

X₁₉ is an amino acid of the general structure

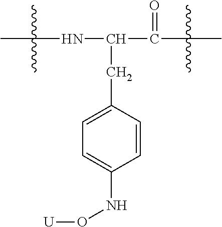

wherein U is an amino acid or a hydroxyl acid and O is an N-alkylated amino acid;

X₄₉ is threonine or a threonine-glutamic acid-glutamic acid tripeptide; and

R₁₃ and R₁₄ are independently COOH or CONH₂. In one embodiment, an IGF^{B16B17} derivative peptide prodrug analog is provided comprising an A chain sequence of GIVDECCFRSCDLRRLEMX₁₉CA-R₁₃ (SEQ ID NO: 22) and a B chain sequence of FVNQTLCGAELVDALYLVCGDRGFYFNKPT-R₁₄ (SEQ ID NO: 72), GPETLCGAELVDALYLVCGDRGFYFNKPT-R₁₄ (SEQ ID NO: 11) or AYRPSETLCGGELVDTLYLVCGDRGFYFSRPA-R₁₄ (SEQ ID NO: 12) wherein

X₁₉ is an amino acid of the general structure

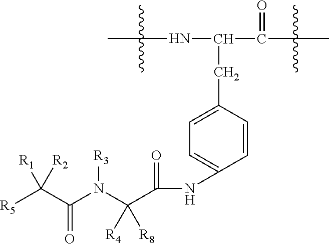

wherein R₁ is selected from the group consisting of H and C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, and CH₂(C₅-C₉ heteroaryl), or R₁ and R₂ together with the atoms to which they are attached form a C₃-C₆ cycloalkyl;

R₃ is selected from the group consisting of C₁-C₈ alkyl, (C₁-C₄ alkyl)OH, (C₁-C₄ alkyl)NH₂, (C₁-C₄ alkyl)SH, and (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring;

R₇ is selected from the group consisting of H and OH; and

R₈ is H; and

R₁₃ and R₁₄ are independently COOH or CONH₂.

The substituents of the dipeptide prodrug element, and its site of attachment to the IGF^{B16B17} derivative peptide, can be selected to provide the desired half life of a prodrug derivative of the IGF^{B16B17} derivative peptides disclosed herein. For example, when a dipeptide prodrug element comprising the structure:

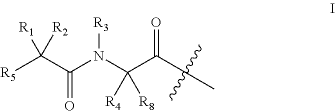

is linked to the alpha amino group of the N-terminal amino acid of the IGF^{B16B17} derivative peptide A or B chain, compounds having a t_{1/2} of about 1 hour in PBS under physiological conditions are provided when

R₁ and R₂ are independently C₁-C_{B} alkyl or aryl; or R₁ and R₂ are linked through —(CH₂)ₚ—, wherein p is 2-9;

R₃ is C₁-C₁₈ alkyl;

R₄ and R₈ are each hydrogen; and

R₅ is an amine.

In other embodiments, prodrugs linked at the N-terminus and having a t_{1/2} of, e.g., about 1 hour comprise a dipeptide prodrug element with the structure:

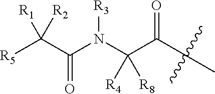

wherein

R₁ and R₂ are independently C₁-C₁₈ alkyl or (C₀-C₁ alkyl)(C₆-C₁₀ aryl)R₇; or R₁ and R₂ are linked through —(CH₂)ₚ, wherein p is 2-9;

R₃ is C₁-C₁₈ alkyl;

R₄ and R₈ are each hydrogen;

R₅ is NH₂;

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo; and R₈ is H.

Alternatively, in one embodiment an IGF^{B16B17} derivative peptide prodrug analog is provided wherein the dipeptide prodrug is linked to the alpha amino group of the N-terminal amino acid of the IGF^{B16B17} derivative peptide A or B chain, and the prodrug has a t_{1/2} between about 6 to about 24 hours in PBS under physiological conditions. In one embodiment an IGF^{B16B17} derivative peptide prodrug analog having a t_{1/2} between about 6 to about 24 hours in PBS under physiological conditions is provided wherein the prodrug element has the structure of formula I and

R₁ and R₂ are independently selected from the group consisting of hydrogen, C₁-C₁₈ alkyl and aryl, or R₁ and R₂ are linked through —(CH₂)ₚ—, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and aryl; and

R₅ is an amine, with the proviso that both R₁ and R₂ are not hydrogen and provided that one of R₄ or R₈ is hydrogen.

In a further embodiment an IGF^{B16B17} derivative peptide prodrug analog is provided wherein the dipeptide prodrug is linked to the alpha amino group of the N-terminal amino acid of the IGF^{B16B17} derivative peptide A or B chain, and the prodrug has a t_{1/2} between about 72 to about 168 hours in PBS under physiological conditions.

In one embodiment an IGF^{B16B17} derivative peptide prodrug analog having a t_{1/2} between about 72 to about 168 hours in PBS under physiological conditions is provided wherein the prodrug element has the structure of Formula I and

R₁ is selected from the group consisting of hydrogen, C₁-C₈ alkyl and aryl;

R₂ is H;

R₃ is C₁-C₁₈ alkyl;

R₄ and R₈ are each hydrogen; and

R₅ is an amine or N-substituted amine or a hydroxyl;

with the proviso that, if R₁ is alkyl or aryl, then R₁ and R₅ together with the atoms to which they are attached form a 4-11 heterocyclic ring.

In some embodiments, prodrugs having the dipeptide prodrug element linked to the N-terminal alpha amino acid of the IGF^{B16B17} derivative A chain or B chain peptide and having a t_{1/2}, e.g., between about 12 to about 72 hours, or in some embodiments between about 12 to about 48 hours, comprise a dipeptide prodrug element with the structure:

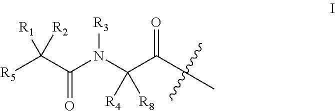

wherein R₁ and R₂ are independently selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇, or R₁ and R₂ are linked through (CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and (C₀-C₄ alkyl)(C₆-C₁₀ aryl)R₇;

R₅ is NH₂; and

R₇ is selected from the group consisting of H, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that both R₁ and R₂ are not hydrogen and provided that at least one of R₄ or R₈ is hydrogen.

In some embodiments, prodrugs having the dipeptide prodrug element linked to the N-terminal amino acid of the IGF^{B16B17} derivative A chain or B chain peptide and having a t_{1/2}, e.g., between about 12 to about 72 hours, or in some embodiments between about 12 to about 48 hours, comprise a dipeptide prodrug element with the structure:

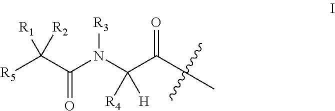

wherein R₁ and R₂ are independently selected from the group consisting of hydrogen, C₁-C₈ alkyl and (C₁-C₄ alkyl)NH₂, or R₁ and R₂ are linked through (CH₂)ₚ, wherein p is 2-9;

R₃ is C₁-C₈ alkyl or R₃ and R₄ together with the atoms to which they are attached form a 4-6 heterocyclic ring;

R₄ is selected from the group consisting of hydrogen and C₁-C₈ alkyl; and

R₅ is NH₂;

with the proviso that both R₁ and R₂ are not hydrogen.

In other embodiments, prodrugs having the dipeptide prodrug element linked to the N-terminal amino acid of the IGF^{B16B17} derivative A chain or B chain peptide and having a t_{1/2}, e.g., between about 12 to about 72 hours, or in some embodiments between about 12 to about 48 hours, comprise a dipeptide prodrug element with the structure:

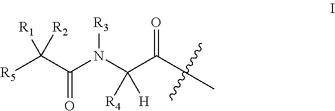

wherein

R₁ and R₂ are independently selected from the group consisting of hydrogen, C₁-C₈ alkyl and (C₁-C₄ alkyl)NH₂;

R₃ is C₁-C₆ alkyl;

R₄ is hydrogen; and

R₅ is NH₂;

with the proviso that both R₁ and R₂ are not hydrogen.

In some embodiments, prodrugs having the dipeptide prodrug element linked to the N-terminal amino acid of the IGF^{B16B17} derivative A chain or B chain peptide and having a t_{1/2}, e.g., between about 12 to about 72 hours, or in some embodiments between about 12 to about 48 hours, comprise a dipeptide prodrug element with the structure:

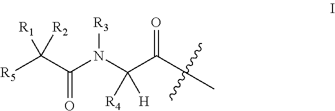

wherein

R₁ and R₂ are independently selected from the group consisting of hydrogen and C₁-C₈ alkyl, (C₁-C₄ alkyl)NH₂, or R₁ and R₂ are linked through (CH₂)ₚ, wherein p is 2-9;

R₃ is C₁-C₈ alkyl;

R₄ is (C₀-C₄ alkyl)(C₆-C₁₀ aryl)R₇;

R₅ is NH₂; and

R₇ is selected from the group consisting of hydrogen, C₁-C₈ alkyl and (C₀-C₄ alkyl)OH;

with the proviso that both R₁ and R₂ are not hydrogen.

In addition a prodrug having the dipeptide prodrug element linked to the N-terminal alpha amino acid of the IGF^{B16B17} derivative peptide and having a t_{1/2}, e.g., of about 72 to about 168 hours is provided wherein the dipeptide prodrug element has the structure:

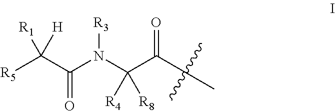

wherein R₁ is selected from the group consisting of hydrogen, C₁-C₈ alkyl and (C₀-C₄ alkyl)(C₆-C₁₀ aryl)R₇;

R₃ is C₁-C₁₈ alkyl;

R₄ and R₈ are each hydrogen;

R₅ is NHR₆ or OH;

R₆ is H, C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that, if R₁ is alkyl or (C₀-C₄ alkyl)(C₆-C₁₀ aryl)R₇, then R₁ and

R₅ together with the atoms to which they are attached form a 4-11 heterocyclic ring.

In some embodiments the dipeptide prodrug element is linked to a side chain amine of an internal amino acid of the IGF^{B16B17} derivative peptide. In this embodiment prodrugs having a t_{1/2}, e.g., of about 1 hour have the structure:

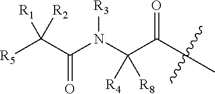

wherein

R₁ and R₂ are independently C₁-C₈ alkyl or (C₀-C₄ alkyl)(C₆-C₁₀ aryl)R₇; or R₁ and R₂ are linked through —(CH₂)ₚ—, wherein p is 2-9;

R₃ is C₁-C₁₈ alkyl;

R₄ and R₈ are each hydrogen;

R₅ is NH₂; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

Furthermore, prodrugs having a t_{1/2}, e.g., between about 6 to about 24 hours and having the dipeptide prodrug element linked to an internal amino acid side chain comprise a dipeptide prodrug element with the structure:

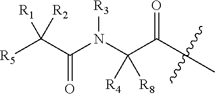

wherein R₁ and R₂ are independently selected from the group consisting of hydrogen, C₁-C₈ alkyl, and (C₀-C₄ alkyl)(C₆-C₁₀ aryl)R₇, or R₁ and R₂ are linked through —(CH₂)ₚ—, wherein p is 2-9;

R₃ is C₁-C₁₈ alkyl or R₃ and R₄ together with the atoms to which they are attached form a 4-12 heterocyclic ring;

R₄ and R₈ are independently hydrogen, C₁-C₁₈ alkyl or (C₀-C₄ alkyl)(C₆-C₁₀ aryl)R₇;

R₅ is NHR₆;

R₆ is H or C₁-C₈ alkyl, or R₆ and R₂ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that both R₁ and R₂ are not hydrogen and provided that at least one of R₄ or R₈ is hydrogen.

In addition a prodrug having a t_{1/2}, e.g., of about 72 to about 168 hours and having the dipeptide prodrug element linked to a internal amino acid side chain of the IGF^{B16B17} derivative peptide is provided wherein the dipeptide prodrug element has the structure:

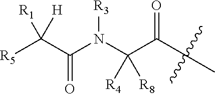

wherein R₁ is selected from the group consisting of hydrogen, C₁-C₁₈ alkyl and (C₀-C₄ alkyl)(C₆-C₁₀ aryl)R₇;

R₃ is C₁-C₁₈ alkyl;

R₄ and R₈ are each hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo; with the proviso that, if R₁ and R₂ are both independently an alkyl or (C₀-C₄ alkyl)(C₆-C₁₀ aryl)R₇, either R₁ or R₂ is linked through (CH₂)ₚ to R₅, wherein p is 2-9.

In some embodiments the dipeptide prodrug element is linked to a side chain amine of an internal amino acid of the IGF^{B16B17} derivative peptide wherein the internal amino acid comprises the structure of Formula III

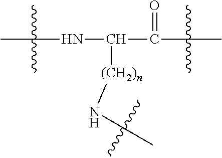

wherein

n is an integer selected from 1 to 4. In some embodiments n is 3 or 4 and in some embodiments the internal amino acid is lysine. In some embodiments the dipeptide prodrug element is linked to a primary amine on a side chain of an amino acid located at position 28, or 29 of the B-chain of the IGF^{B16B17} derivative peptide.

In embodiments where the dipeptide prodrug element of formula I is linked to an amino substituent of an aryl group of an aromatic amino acid, prodrug, the substituents of the prodrug element can be selected to provide the desired time of activation. For example, the half life of a prodrug derivative of any of the IGF^{B16B17} derivative peptides disclosed herein comprising an amino acid of the structure of Formula II:

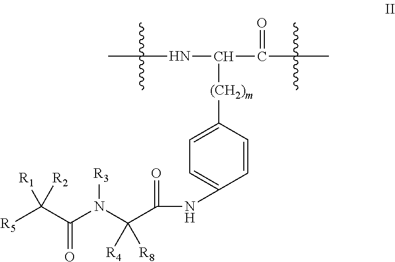

wherein m is an integer from 0 to 3, can be selected by altering the substituents of R₁, R₂, R₃, R₄, R₅, and R₈. In one embodiment the amino acid of formula II is present at an amino acid corresponding to position A19, B16 or B25 of native insulin, and in one specific example the amino acid of formula II is located at position A19 of the IGF^{B16B17} derivative peptide, and m is 1. In one embodiment an IGF^{B16B17} derivative peptide prodrug analog comprising the structure of Formula II and having a t½ of about 1 hour in PBS under physiological conditions is provided. In one embodiment the IGF^{B16B17} derivative peptide prodrug analog having a t½ of about 1 hour in PBS under physiological conditions comprises the structure of formula II wherein,

R₁ and R₂ are independently C₁-C₁₈ alkyl or aryl;

R₃ is C₁-C₁₈ alkyl or R₃ and R₄ together with the atoms to which they are attached form a 4-12 heterocyclic ring;

R₄ and R_{g} are independently selected from the group consisting of hydrogen, C₁-C₁₈ alkyl and aryl; and

R₅ is an amine or a hydroxyl. In one embodiment m is 1.

In one embodiment, the dipeptide prodrug element is linked to the IGF^{B16B17} derivative peptide via an amine present on an aryl group of an aromatic amino acid of the IGF^{B16B17} derivative peptide, wherein prodrugs having a t_{1/2}, e.g., of about 1 hour have a dipeptide structure of:

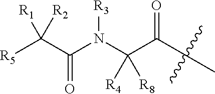

wherein R₁ and R₂ are independently C₁-C₁₈ alkyl or (C₀-C₄ alkyl)(C₆-C₁₀ aryl)R₇;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₁₈ alkyl and (C₀-C₄ alkyl)(C₆-C₁₀ aryl)R₇;

R₅ is NH₂ or OH; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In another embodiment an IGF^{B16B17} derivative peptide prodrug analog comprising the structure of Formula II, wherein m is an integer from 0 to 3 and having a t½ of about 6 to about 24 hours in PBS under physiological conditions, is provided. In one embodiment where the IGF^{B16B17} derivative peptide prodrug having a t½ of about 6 to about 24 hours in PBS under physiological conditions comprises the structure of formula II wherein,

R₁ is selected from the group consisting of hydrogen, C₁-C₁₈ alkyl and aryl, or R₁ and R₂ are linked through —(CH₂)ₚ—, wherein p is 2-9;

R₃ is C₁-C₁₈ alkyl or R₃ and R₄ together with the atoms to which they are attached form a 4-6 heterocyclic ring;

R₄ and R₈ are independently selected from the group consisting of hydrogen, C₁-C₁₈ alkyl and aryl; and

R₅ is an amine or N-substituted amine. In one embodiment m is 1.

In one embodiment, prodrugs having the dipeptide prodrug element linked via an aromatic amino acid and having a t_{1/2}, e.g., of about 6 to about 24 hours are provided wherein the dipeptide comprises a structure of:

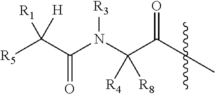

wherein

R₁ is selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇;

R₃ is C₁-C₁₈ alkyl or R₃ and R₄ together with the atoms to which they are attached form a 4-6 heterocyclic ring;

R₄ and R₈ are independently selected from the group consisting of hydrogen, C₁-C₁₈ alkyl and (C₀-C₄ alkyl)(C₆-C₁₀ aryl)R₇;

R₅ is NHR₆;

R₆ is H, C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In another embodiment an IGF^{B16B17} derivative peptide prodrug analog comprising the structure of Formula II, wherein m is an integer from 0 to 3 and having a t½ of about 72 to about 168 hours in PBS under physiological conditions, is provided. In one embodiment where the IGF^{B16B17} derivative peptide prodrug analog having a t½ of about 72 to about 168 hours in PBS under physiological conditions comprises the structure of formula II wherein,

R₁ and R₂ are independently selected from the group consisting of hydrogen, C₁-C₈ alkyl and aryl;

R₃ is C₁-C₁₈ alkyl or R₃ and R₄ together with the atoms to which they are attached form a 4-6 heterocyclic ring;

R₄ and R₈ are each hydrogen; and

R₅ is selected from the group consisting of amine, N-substituted amine and hydroxyl. In one embodiment m is 1.

In one embodiment, prodrugs having the dipeptide prodrug element linked via an aromatic amino acid and having a t_{1/2}, e.g., of about 72 to about 168 hours are provided wherein the dipeptide comprises a structure of:

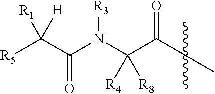

wherein R₁ and R₂ are independently selected from the group consisting of hydrogen, C₁-C₈ alkyl, (C₁-C₄ alkyl)COOH, and (C₀-C₄ alkyl)(C₆-C₁₀ aryl)R₇, or R₁ and R₅ together with the atoms to which they are attached form a 4-11 heterocyclic ring;

R₃ is C₁-C₁₈ alkyl or R₃ and R₄ together with the atoms to which they are attached form a 4-6 heterocyclic ring;

R₄ is hydrogen or forms a 4-6 heterocyclic ring with R₃;

R₈ is hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In accordance with one embodiment a single-chain IGF^{B16B17} derivative peptide prodrug analog is provided wherein the carboxy terminus of an IGF analog B chain, as disclosed herein, is covalently linked to the N-terminus of an IGF analog A chain, as disclosed herein, and further wherein a dipeptide prodrug moiety having the general structure:

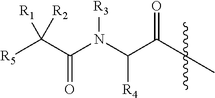

is covalently bound at the N-terminus of the peptide, or at the side chain of an amino acid corresponding to positions A19, B16 or B25 of the respective native insulin A chain or B chain, via an amide bond. In accordance with one embodiment the single-chain IGF^{B16B17} derivative peptide comprises a compound of the formula: B-P-A, wherein: B represents an IGF analog

B-chain, as disclosed herein, and A represents the A chain of an IGF analog, as disclosed herein, and P represents a linker, including a peptide linker, that covalently joins the A chain to the B chain. In one embodiment the linker is a peptide linker of about 5 to about 18, or about 10 to about 14, or about 4 to about 8, or about 6 amino acids. In one embodiment the B chain is linked to the A chain via peptide linker of 4-12 or 4-8 amino acids.

In one embodiment the single chain insulin analog comprises a compound of the formula: B-P-A, wherein “B” represents an IGF B chain comprising the sequence GPETLCGAELVDALYLVCGDRGFYFNKPT-R₁₄ (SEQ ID NO: 11), “A” represents an IGF A chain comprising the sequence GIVDECCFRSCDLRRLEMX₁₉CA-R₁₃ (SEQ ID NO: 22) wherein

X₁₉ is an amino acid of the general structure

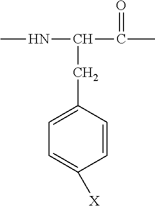

wherein X is selected from the group consisting of OH or NHR₁₀, wherein R₁₀ is a dipeptide comprising the general structure:

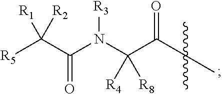

R₁ is selected from the group consisting of H and C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl(SCH₃, (C₁-C₄ alkyl)CONH₂, (C₁-C₄ alkyl)COOH, (C₁-C₄ alkyl)NH₂, (C₁-C₄ alkyl)NHC(NH₂⁺) NH₂, (C₀-C₄ alkyl)(C₃-C₆ cycloalkyl), (C₀-C₄ alkyl)(C₆-C₁₀ aryl)R₇, and CH₂(C₅-C₉ heteroaryl), or R₁ and R₂ together with the atoms to which they are attached form a C₃-C₆ cycloalkyl;

R₃ is selected from the group consisting of C₁-C₈ alkyl, (C₁-C₄ alkyl)OH, (C₁-C₄ alkyl)NH₂, (C₁-C₄ alkyl)SH, and (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring;

R₇ is selected from the group consisting of H and OH; and

R₈ is H; and

R₁₃ and R₁₄ are independently COOH or CONH₂. The present invention also encompasses any combination of IGF analog A chain and B chain peptides, as disclosed herein, linked together as a single chain peptide of the formula B-P-A. In accordance with one embodiment R₁₀ is a dipeptide comprising the general structure of Formula I:

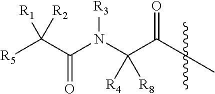

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃ (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo, with the proviso that when the dipeptide of Formula I is linked to an N-terminal amine and R₄ and R₃ together with the atoms to which they are attached form a 4, 5 or 6 member heterocyclic ring, then both R₁ and R₂ are not hydrogen.

In accordance with one embodiment the peptide linker, “P”, is 5 to 18 amino acids in length and comprises a sequence selected from the group consisting of: Gly-Gly-Gly-Pro-Gly-Lys-Arg (SEQ ID NO: 27), Gly-Tyr-Gly-Ser-Ser-Ser-Arg-Arg-Ala-Pro-Gln-Thr (SEQ ID NO: 28), Arg-Arg-Gly-Pro-Gly-Gly-Gly (SEQ ID NO: 37), Gly-Gly-Gly-Gly-Gly-Lys-Arg (SEQ ID NO: 29), Arg-Arg-Gly-Gly-Gly-Gly-Gly (SEQ ID NO: 30), Gly-Gly-Ala-Pro-Gly-Asp-Val-Lys-Arg (SEQ ID NO: 31), Arg-Arg-Ala-Pro-Gly-Asp-Val-Gly-Gly (SEQ ID NO: 32), Gly-Gly-Tyr-Pro-Gly-Asp-Val-Lys-Arg (SEQ ID NO: 33), Arg-Arg-Tyr-Pro-Gly-Asp-Val-Gly-Gly (SEQ ID NO: 34), Gly-Gly-His-Pro-Gly-Asp-Val-Lys-Arg (SEQ ID NO: 35) and Arg-Arg-His-Pro-Gly-Asp-Val-Gly-Gly (SEQ ID NO: 36). In one embodiment the peptide linker is 7 to 12 amino acids in length and comprises the sequence Gly-Gly-Gly-Pro-Gly-Lys-Arg (SEQ ID NO: 27) or Gly-Tyr-Gly-Ser-Ser-Ser-Arg-Arg-Ala-Pro-Gln-Thr (SEQ ID NO: 28).

In a further embodiment the peptide linker comprises a sequence selected from the group consisting of AGRGSGK (SEQ ID NO: 40), AGLGSGK (SEQ NO: 41), AGMGSGK (SEQ ID NO: 42), ASWGSGK (SEQ ID NO: 43), TGLGSGQ (SEQ ID NO: 44), TGLGRGK (SEQ ID NO: 45), TGLGSGK (SEQ ID NO: 46), HGLYSGK (SEQ ID NO: 47), KGLGSGQ (SEQ ID NO: 48), VGLMSGK (SEQ ID NO: 49), VGLSSGQ (SEQ ID NO: 50), VGLYSGK (SEQ ID NO: 51). VGLSSGK (SEQ ID NO: 52), VGMSSGK (SEQ ID 53), VWSSSGK (SEQ ID NO: 54), VGSSSGK (SEQ ID NO: 55), VGMSSGK (SEQ ID NO: 56), TGLGSGR (SEQ ID NO: 57), TGLGKGQ (SEQ ID NO: 58), KGLSSGQ (SEQ ID NO: 59), VKLSSGQ (SEQ ID NO: 60), VGLKSGQ (SEQ ID NO: 61), TGLGKGQ (SEQ ID NO: 62), SRVSRRSR (SEQ ID NO: 79), GYGSSSRRAPQT (SEQ ID NO: 28) and VGLSKGQ (SEQ ID NO: 63). In one embodiment the linker comprises GSSSRRAP (SEQ ID NO: 80) or SRVSRRSR (SEQ ID NO: 79).

In one embodiment the single-chain insulin analog comprises the amino acid sequence: His-Leu-Cys-Gly-Ala-Glu-Leu-Val-Glu-Ala-Leu-Tyr-Leu-Val-Cys-Gly-Asp-Ala- Gly-Phe-Tyr-Phe-Asn-Lys-Pro-Thr-Gln-Pro-Leu-Ala-Leu-Glu-Gly-Ser-Leu-Gln- Lys-Arg-Gly-Ile-Val-Asp-Glu-Cys-Cys-His-Ala-Ser-Cys-Asp-Leu-Arg-Arg-Leu- Glu-Met-Xaa-Cys-Asn (SEQ ID NO: 38) or Thr-Leu-Cys-Gly-Ala-Glu-Leu-Val-Asp-Ala-Leu-Tyr-Leu-Val-Cys-Gly-Asp-Arg- Gly-Phe-Tyr-Phe-Asn-Lys-Pro-Thr-Gln-Pro-Leu-Ala-Leu-Glu-Gly-Ser-Leu-Gln- Lys-Arg-Gly-Ile-Val-Asp-Glu-Cys-Cys-Phe-Arg-Ser-Cys-Asp-Leu-Arg-Arg-Leu- Glu-Met-Xaa-Cys-Ala (SEQ ID NO: 39) wherein Xaa is an amino acid of the general structure:

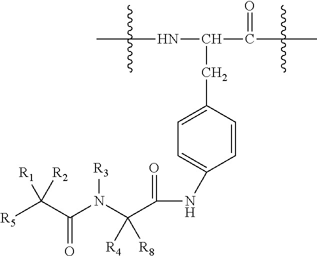

wherein

R₁ is selected from the group consisting of H and C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, and CH₂(C₅-C₉ heteroaryl), or R₁ and R₂ together with the atoms to which they are attached form a C₃-C₆ cycloalkyl;

R₃ is selected from the group consisting of C₁-C₈ alkyl, (C₁-C₄ alkyl)OH, (C₁-C₄ alkyl)NH₂, (C₁-C₄ alkyl)SH, and (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring;

R₇ is selected from the group consisting of H and OH; and

R₈ is H.

The prodrugs disclosed herein can be further modified to improve the peptide's solubility in aqueous solutions at physiological pH, while enhancing the effective duration of the peptide by preventing renal clearance of the peptide. Peptides are easily cleared because of their relatively small molecular size when compared to plasma proteins. Increasing the molecular weight of a peptide above 40 kDa exceeds the renal threshold and significantly extends duration in the plasma. Accordingly, in one embodiment the peptide prodrugs are further modified to comprise a covalently linked hydrophilic moiety.

In one embodiment the hydrophilic moiety is a plasma protein, polyethylene oxide chain or the Fc portion of an immunoglobin. Therefore, in one embodiment the presently disclosed IGF^{B16B17} derivative peptide and prodrug derivatives thereof are further modified to comprise one or more hydrophilic groups covalently linked to the side chains of amino acids.

In accordance with one embodiment the insulin prodrugs disclosed herein are further modified by linking a hydrophilic moiety to either the N-terminal amino acid of the B chain or to the side chain of a lysine amino acid (or other suitable amino acid) located at the carboxy terminus of the B chain, including for example, at position 28 of SEQ ID NO: 11. In one embodiment a single-chain insulin prodrug analog is provided wherein one of the amino acids of the peptide linker is modified by linking a hydrophilic moiety to the side chain of the peptide linker. In one embodiment the modified amino acid is cysteine, lysine or acetyl phenylalanine. In one embodiment the peptide linker is selected from the group consisting of TGLGSGQ (SEQ ID NO: 44), VGLSSGQ (SEQ ID NO: 50), VGLSSGK (SEQ ID NO: 52), TGLGSGR (SEQ ID NO: 57), TGLGKGQ (SEQ ID NO: 58), KGLSSGQ (SEQ ID NO: 59), VKLSSGQ (SEQ ID NO: 60), VGLKSGQ (SEQ ID NO: 61), TGLGKGQ (SEQ ID NO: 62), SRVSRRSR (SEQ ID NO: 79), GYGSSSRRAPQT (SEQ ID NO: 28) and VGLSKGQ (SEQ ID NO: 63) and the hydrophilic moiety (e.g., polyethylene glycol) is linked to the lysine side chain of the peptide linker.

In another embodiment the IGF^{B16B17} derivative peptides, and their prodrug derivatives, disclosed herein are further modified by the addition of a modified amino acid to the carboxy or amino terminus of the A chain or B chain of the IGF^{B16B17} derivative peptide, wherein the added amino acid is modified to comprise a hydrophilic moiety linked to the amino acid. In one embodiment the amino acid added to the C-terminus is a modified cysteine, lysine or acetyl phenylalanine. In one embodiment the hydrophilic moiety is selected from the group consisting of a plasma protein, polyethylene oxide chain and an Fc portion of an immunoglobin.

In one embodiment the hydrophilic group is a polyethylene oxide chain, and in one embodiment two or more polyethylene oxide chains are covalently attached to two or more amino acid side chains of the IGF^{B16B17} derivative peptide. In accordance with one embodiment the hydrophilic moiety is covalently attached to an amino acid side chain of an IGF^{B16B17} derivative peptide prodrug disclosed herein at a position corresponding to A10, B28, B29 and the C-terminus or N-terminus of native insulin. For IGF^{B16B17} derivative peptides and their prodrug derivatives having multiple polyethylene oxide chains, the polyethylene oxide chains can be attached at the N-terminal amino acid of the B chain or to the side chain of a lysine amino acid located at the carboxy terminus of the B chain, or by the addition of a single amino acid at the C-terminus of the peptide wherein the added amino acid has a polyethylene oxide chain linked to its side chain. In accordance with one embodiment the polyethylene oxide chain or other hydrophilic moiety is linked to the side chain of one of the two amino acids comprising the dipeptide prodrug element. In one embodiment the dipeptide prodrug element comprises a lysine (in the D or L stereoisomer configuration) with a polyethylene oxide chain attached to the side chain amine of the lysine.

In accordance with one embodiment, the IGF^{B16B17} derivative peptides, and prodrug derivatives thereof, disclosed herein are further modified by amino acid substitutions, wherein the substituting amino acid comprises a side chain suitable for crosslinking with hydrophilic moieties, including for example, polyethylene glycol. In one embodiment the amino acid at the position of the IGF^{B16B17} derivative peptide where the hydrophilic moiety is to be linked is substituted (or added at the C-terminus) with a natural or synthetic amino acid to introduce, or allow for ease in attaching, the hydrophilic moiety. For example, in one embodiment a native amino acid at a position corresponding to A5, A8, A9, A10, A12, A14, A15, A17, A18, B1, B2, B3, B4, B5, B13, B14, B17, B21, B22, B26, B27, B28, B29 and B30 of native insulin is substituted with a lysine, cysteine or acetyl phenylalanine residue (or a lysine, cysteine or acetyl phenylalanine residue is added to the C-terminus) to allow for the covalent attachment of a polyethylene oxide chain.

In one embodiment the IGF^{B16B17} derivative peptide, or prodrug derivative thereof, has a single cysteine residue added to the amino or carboxy terminus of the B chain, or the insulin prodrug analog is substituted with at least one cysteine residue, wherein the side chain of the cysteine residue is further modified with a thiol reactive reagent, including for example, maleimido, vinyl sulfone, 2-pyridylthio, haloalkyl, and haloacyl. These thiol reactive reagents may contain carboxy, keto, hydroxyl, and ether groups as well as other hydrophilic moieties such as polyethylene glycol units. In an alternative embodiment, the IGF^{B16B17} derivative peptide, or prodrug derivative thereof, has a single lysine residue added to the amino or carboxy terminus of the B chain, or the IGF^{B16B17} derivative peptide prodrug analog is substituted with lysine, and the side chain of the substituting lysine residue is further modified using amine reactive reagents such as active esters (succinimido, anhydride, etc) of carboxylic acids or aldehydes of hydrophilic moieties such as polyethylene glycol.

### Linkage of Hydrophilic Moieties

In another embodiment the solubility of the IGF^{B16B17} derivative peptides disclosed herein are enhanced by the covalent linkage of a hydrophilic moiety to the peptide. Hydrophilic moieties can be attached to the IGF^{B16B17} derivative peptides under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane and 5-pyridyl. If attached to the peptide by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1, 3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof.

The hydrophilic moiety, e.g., polyethylene glycol chain in accordance with some embodiments has a molecular weight selected from the range of about 500 to about 40,000 Daltons. In one embodiment the hydrophilic moiety, e.g. PEG, has a molecular weight selected from the range of about 500 to about 5,000 Daltons, or about 1,000 to about 5,000 Daltons. In another embodiment the hydrophilic moiety, e.g., PEG, has a molecular weight of about 10,000 to about 20,000 Daltons. In yet other exemplary embodiments the hydrophilic moiety, e.g., PEG, has a molecular weight of about 20,000 to about 40,000 Daltons.

In one embodiment dextrans are used as the hydrophilic moiety. Dextrans are polysaccharide polymers of glucose subunits, predominantly linked by α1-6 linkages. Dextran is available in many molecular weight ranges, e.g., about 1 kD to about 100 kD, or from about 5, 10, 15 or 20 kD to about 20, 30, 40, 50, 60, 70, 80 or 90 kD.

Linear or branched polymers are contemplated. Resulting preparations of conjugates may be essentially monodisperse or polydisperse, and may have about 0.5, 0.7, 1, 1.2, 1.5 or 2 polymer moieties per peptide.

In those embodiments wherein the IGF^{B16B17} derivative peptide, or prodrug derivative thereof, comprises a polyethylene glycol chain, the polyethylene glycol chain may be in the form of a straight chain or it may be branched. In accordance with one embodiment the polyethylene glycol chain has an average molecular weight selected from the range of about 20,000 to about 60,000 Daltons. Multiple polyethylene glycol chains can be linked to the IGF^{B16B17} derivative peptide to provide an insulin analog with optimal solubility and blood clearance properties. In one embodiment the IGF^{B16B17} derivative peptide, or prodrug derivative thereof, is linked to a single polyethylene glycol chain that has an average molecular weight selected from the range of about 20,000 to about 60,000 Daltons. In another embodiment the IGF^{B16B17} derivative peptide, or prodrug derivative thereof, is linked to two polyethylene glycol chains wherein the combined average molecular weight of the two chains is selected from the range of about 40,000 to about 80,000 Daltons. In one embodiment a single polyethylene glycol chain having an average molecular weight of 20,000 or 60,000 Daltons is linked to the IGF^{B16B17} derivative peptide, or prodrug derivative thereof. In another embodiment a single polyethylene glycol chain is linked to the IGF^{B16B17} derivative peptide, or prodrug derivative thereof, and has an average molecular weight selected from the range of about 40,000 to about 50,000 Daltons. In one embodiment two polyethylene glycol chains are linked to the IGF^{B16B17} derivative peptide, or prodrug derivative thereof, wherein the first and second polyethylene glycol chains each have an average molecular weight of 20,000 Daltons. In another embodiment two polyethylene glycol chains are linked to the IGF^{B16B17} derivative peptide, or prodrug derivative thereof, wherein the first and second polyethylene glycol chains each have an average molecular weight of 40,000 Daltons.

In a further embodiment an IGF^{B16B17} derivative peptide, or prodrug derivative thereof, comprising two or more polyethylene glycol chains covalently bound to the peptide is provided, wherein the total molecular weight of the polyethylene glycol chains is about 40,000 to about 60,000 Daltons. In one embodiment the pegylated IGF^{B16B17} derivative peptide, or prodrug derivative thereof, comprises a polyethylene glycol chain linked to one or more amino acids selected from the N-terminus of the B chain and/or position 28 of SEQ ID NO: 11, wherein the combined molecular weight of the PEG chain(s) is about 40,000 to about 80,000 Daltons.

In another embodiment the IGF^{B16B17} derivative peptides disclosed herein are further modified by the addition of a modified amino acid to the carboxy terminus of the B chain of the IGF^{B16B17} derivative peptide, wherein the C-terminally added amino acid is modified to comprise a hydrophilic moiety linked to the amino acid. In one embodiment the amino acid added to the C-terminus is a modified cysteine, lysine or acetyl phenylalanine. In one embodiment the hydrophilic moiety is selected from the group consisting of a plasma protein, polyethylene oxide chain and an Fc portion of an immunoglobin.

In accordance with one embodiment, an IGF^{B16B17} derivative peptide, or prodrug/depot derivative thereof, are fused to an accessory peptide which is capable of forming an extended conformation similar to chemical PEG (e.g., a recombinant PEG (rPEG) molecule), such as those described in International Patent Application Publication No. WO2009/023270 and U.S. Patent Application Publication No. US2008/0286808. The rPEG molecule is not polyethylene glycol. The rPEG molecule in some aspects is a polypeptide comprising one or more of glycine, serine, glutamic acid, aspartic acid, alanine, or proline. In some aspects, the rPEG is a homopolymer, e.g., poly-glycine, poly-serine, poly-glutamic acid, poly-aspartic acid, poly-alanine, or poly-proline. In other embodiments, the rPEG comprises two types of amino acids repeated, e.g., poly(Gly-Ser), poly(Gly-Glu), poly(Gly-Ala), poly(Gly-Asp), poly(Gly-Pro), poly(Ser-Glu), etc. In some aspects, the rPEG comprises three different types of amino acids, e.g., poly(Gly-Ser-Glu). In specific aspects, the rPEG increases the half-life of the IGF^{B16B17} derivative peptide. In some aspects, the rPEG comprises a net positive or net negative charge. The rPEG in some aspects lacks secondary structure. In some embodiments, the rPEG is greater than or equal to 10 amino acids in length, and in some embodiments is about 40 to about 50 amino acids in length. The accessory peptide in some aspects is fused to the N- or C-terminus of the peptide of the invention through a peptide bond or a proteinase cleavage site, or is inserted into the loops of the peptide of the invention. The rPEG in some aspects comprises an affinity tag or is linked to a PEG that is greater than 5 kDa. In some embodiments, the rPEG confers the peptide of the invention with an increased hydrodynamic radius, serum half-life, protease resistance, or solubility and in some aspects confers the peptide with decreased immunogenicity.

In accordance with one embodiment, an IGF^{B16B17} derivative peptide, or prodrug derivative thereof, is provided wherein a plasma protein has been covalently linked to an amino acid side chain of the peptide to improve the solubility, stability and/or pharmacokinetics of the insulin prodrug analog. For example, serum albumin can be covalently bound to the IGF^{B16B17} derivative peptide, or prodrug derivative thereof, presented herein. In one embodiment the plasma protein is covalently bound to the N-terminus of the B chain and/or to an amino acid corresponding to position 28 or 29 relative to native insulin (e.g., position 27 of SEQ ID NO: 11).

In accordance with one embodiment, an IGF^{B16B17} derivative peptide, or prodrug derivative thereof, is provided wherein a linear amino acid sequence representing the Fc portion of an immunoglobin molecule has been covalently linked to an amino acid side chain to improve the solubility, stability and/or pharmacokinetics of the IGF^{B16B17} derivative peptide, or prodrug derivative thereof. For example, the amino acid sequence representing the Fc portion of an immunoglobin molecule can be covalently bound to the amino or carboxy terminus of the A chain, or the amino or carboxy terminus of an A chain that has been terminally extended. The Fc portion is typically one isolated from IgG, but the Fc peptide fragment from any immunoglobin should function equivalently.

In one specific embodiment, the IGF^{B16B17} derivative peptide, or prodrug derivative thereof, is modified to comprise an alkyl or acyl group by direct alkylation or acylation of an amine, hydroxyl, or thiol of a side chain of an amino acid of the IGF^{B16B17} derivative peptide prodrug analog. In some embodiments, the IGF^{B16B17} derivative peptide prodrug analog is directly acylated through the side chain amine, hydroxyl, or thiol of an amino acid. In some embodiments, acylation is at one or more positions of the IGF^{B16B17} derivative peptide corresponding to positions A10, B28 or B29 of native insulin. In some specific embodiments, the direct acylation of the insulin prodrug analog occurs through the side chain amine, hydroxyl, or thiol of an amino acid present in the carboxy terminal amino acids of the B chain. In one further embodiment the IGF^{B16B17} derivative peptide comprises an acyl group of a carboxylic acid with 1-24 carbon atoms bound to the epsilon-amino group of a Lys present at the corresponding insulin position B28 of SEQ ID NO: 11. In one embodiment a single-chain insulin prodrug analog is provided wherein one of the amino acids of the peptide linker is modified to comprise an acyl group by direct acylation of an amine, hydroxyl, or thiol of a side chain of an amino acid of the peptide linker. In accordance with one embodiment the peptide linker of the single-chain insulin analog is selected from the group consisting of AGRGSGK (SEQ ID NO: 40). AGLOSOK. (SEQ ID NO: 41), AGMGSGK (SEQ ID NO: 42), ASWGSGK (SEQ ID NO: 43), TGLGSGQ (SEQ ID NO: 44), TGLGRGK (SEQ ID NO: 45), TGLGSGK (SEQ ID NO: 46), HGLYSGK (SEQ ID NO: 47), KGLSSGQ (SEQ ID NO: 48), VGLMSGK (SEQ ID NO: 49), VGLSSGQ (SEQ ID NO: 50), VGLYSGK (SEQ ID NO: 51), VGLSSGK (SEQ ID NO: 52), VGMSSGK (SEQ ID NO: 53), VWSSSGK (SEQ ID NO: 54), VGSSSGK (SEQ ID NO: 55), VGMSSGK (SEQ ID NO: 56), TGLGSGR (SEQ ID NO: 57), TGLGKGQ (SEQ ID NO: 58), KGLSSGQ (SEQ ID NO: 59), VKLSSGQ (SEQ ID NO: 60), VGLKSGQ (SEQ ID NO: 61), TGLGKGQ (SEQ ID NO: 62) and VGLSKGQ (SEQ ID NO: 63) wherein at least one lysine residue in the A-chain, in the B-chain or in the connecting peptide has been chemically modified by acylation. In one embodiment the acylating group comprises a 1-5, 10-12 or 12-24 carbon chain.

In accordance with one embodiment the IGF^{B16B17} derivative peptide prodrug analogs as disclosed herein are further modified to link an additional compound to the prodrug dipeptide moiety of the analog. In one embodiment the side chain of an amino acid comprising the dipeptide prodrug element is pegylated, acylated or alkylated. In one embodiment the dipeptide is acylated with a group comprising a 1-5, 10-12 or 12-24 carbon chain. In one embodiment the dipeptide is pegylated with a 40-80 KDa polyethylene glycol chain. In one embodiment the dipeptide prodrug element is pegylated and the IGF^{B16B17} derivative peptide sequence linked to the dipeptide is acylated, including, for example, acylation at the lysine present at A10 or at the C-terminal lysine of the B chain. In accordance with one embodiment a hydrophilic moiety or a sequestering macromolecule is covalently linked to the R₂ side chain of the dipeptide comprising the general structure:

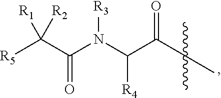

wherein R₂ is selected from the group consisting of (C₁-C₄ alkyl)OH, (C₁-C₄ alkyl)SH, and (C₁-C₄ alkyl)NH₂ wherein the remaining substituents have been defined previously herein. In one embodiment R₂ is (C₃-C₄ alkyl)NH₂. Sequestering macromolecules are known to those skilled in the art and include dextrans and large molecular weight polyethylene oxide chains (e.g., greater than or equal to 40-80 KDa). By linking the sequestering macromolecule to the dipeptide moiety, the prodrug will remain sequestered, while the active IGF^{B16B17} derivative peptide is slowly released based on the kinetics of the cleavage of the dipeptide amide bond.

The present disclosure also encompasses other conjugates in which IGF^{B16B17} derivative peptide prodrug analogs of the invention are linked, optionally via covalent bonding, and optionally via a linker, to a conjugate. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

Exemplary conjugates include but are not limited to a heterologous peptide or polypeptide (including for example, a plasma protein), a targeting agent, an immunoglobulin or portion thereof (e.g. variable region, CDR, or Fc region), a diagnostic label such as a radioisotope, fluorophore or enzymatic label, a polymer including water soluble polymers, or other therapeutic or diagnostic agents. In one embodiment a conjugate is provided comprising an IGF^{B16B17} derivative peptide prodrug analog of the present disclosure and a plasma protein, wherein the plasma protein is selected from the group consisting of albumin, transferin and fibrinogen. In one embodiment the plasma protein moiety of the conjugate is albumin or transferin. In some embodiments, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers are from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

### Conjugates and Fusions

The present disclosure also encompasses other conjugates in which IGF^{B16B17} derivative peptides of the invention are linked, optionally via covalent bonding and optionally via a linker, to a conjugate moiety. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

The peptide can be linked to conjugate moieties via direct covalent linkage by reacting targeted amino acid residues of the peptide with an organic derivatizing agent that is capable of reacting with selected side chains or the N- or C-terminal residues of these targeted amino acids. Reactive groups on the peptide or conjugate include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the peptide indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagines or glutamine, acetylation of the N-terminal amine, and/or amidation or esterification of the C-terminal carboxylic acid group.

Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981).

Exemplary conjugate moieties that can be linked to any of the IGF^{B16B17} derivative peptides described herein include but are not limited to a heterologous peptide or polypeptide (including for example, a plasma protein), a targeting agent, an immunoglobulin or portion thereof (e.g. variable region, CDR, or Fc region), a diagnostic label such as a radioisotope, fluorophore or enzymatic label, a polymer including water soluble polymers, or other therapeutic or diagnostic agents. In one embodiment a conjugate is provided comprising a IGF^{B16B17} derivative peptide disclosed herein and a plasma protein, wherein the plasma protein is selected form the group consisting of albumin, transferin, fibrinogen and globulins.

In some embodiments, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers are from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

As noted above, in some embodiments, the IGF^{B16B17} derivative peptides are conjugated, e.g., fused to an immunoglobulin or portion thereof (e.g. variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC).

For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CH2 domain of a human IgG Fc region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In a related embodiments, the Fc region may comprise one or more native or modified constant regions from an immunoglobulin heavy chain, other than CH1, for example, the CH2 and CH3 regions of IgG and IgA, or the CH3 and CH4 regions of IgE.

Suitable conjugate moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the CH3 domain.

Some conjugate moieties may or may not include FcγR binding site(s). FcγR are responsible for ADCC and CDC. Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005). Amino acid residues involved in IgE receptor binding are described in Sayers et al. (J Biol Chem. 279(34):35320-5, 2004).

Amino acid modifications may be made to the Fc region of an immunoglobulin. Such variant Fc regions comprise at least one amino acid modification in the CH3 domain of the Fc region (residues 342-447) and/or at least one amino acid modification in the CH2 domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591). Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety.

### Linkage of Hydrophilic Moieties

In another embodiment the solubility of the insulin analogs disclosed herein are enhanced by the covalent linkage of a hydrophilic moiety to the peptide. Hydrophilic moieties can be attached to the insulin analogs under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane and 5-pyridyl. If attached to the peptide by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1, 3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof.

### Acylation and Alkylation

In accordance with some embodiments, the IGF^{B16B17} derivative peptides disclosed herein are modified to comprise an acyl group or alkyl group. Acylation or alkylation can increase the half-life of the IGF^{B16B17} derivative peptides in circulation. Acylation or alkylation can advantageously delay the onset of action and/or extend the duration of action at the insulin and/or IGF-1 receptors and/or improve resistance to proteases such as DPP-IV and/or improve solubility. IGF^{B16B17} derivative peptides may be acylated or alkylated at the same amino acid position where a hydrophilic moiety is linked, or at a different amino acid position.

In some embodiments, the invention provides a IGF^{B16B17} derivative peptide modified to comprise an acyl group or alkyl group covalently linked to the amino acid at a position corresponding to A10, B28, B29 of native insulin, or at the C-terminus or N-terminus of the A or B chain. The IGF^{B16B17} derivative peptide may further comprise a spacer between the IGF^{B16B17} derivative peptide amino acid and the acyl group or alkyl group. In some embodiments, the acyl group is a fatty acid or bile acid, or salt thereof, e.g. a C4 to C30 fatty acid, a C8 to C24 fatty acid, cholic acid, a C4 to C30 alkyl, a C8 to C24 alkyl, or an alkyl comprising a steroid moiety of a bile acid. The spacer is any moiety with suitable reactive groups for attaching acyl or alkyl groups. In exemplary embodiments, the spacer comprises an amino acid, a dipeptide, or a tripeptide, or a hydrophilic bifunctional spacer. In some embodiments, the spacer is selected from the group consisting of: Trp, Glu, Asp, Cys and a spacer comprising NH₂(CH₂CH₂O)n(CH₂)mCOOH, wherein m is any integer from 1 to 6 and n is any integer from 2 to 12. Such acylated or alkylated IGF^{B16B17} derivative peptides may also further comprise a hydrophilic moiety, optionally a polyethylene glycol. Any of the foregoing IGF^{B16B17} derivative peptides may comprise two acyl groups or two alkyl groups, or a combination thereof.

Acylation can be carried out at any positions within the IGF^{B16B17} derivative peptide, provided that IGF^{B16B17} derivative peptide insulin agonist activity is retained. The acyl group can be covalently linked directly to an amino acid of the IGF^{B16B17} derivative peptide, or indirectly to an amino acid of the IGF^{B16B17} derivative peptide via a spacer, wherein the spacer is positioned between the amino acid of the IGF^{B16B17} derivative peptide and the acyl group. In a specific aspect of the invention, the IGF^{B16B17} derivative peptide is modified to comprise an acyl group by direct acylation of an amine, hydroxyl, or thiol of a side chain of an amino acid of the IGF^{B16B17} derivative peptide. In some embodiments, the IGF^{B16B17} derivative peptide is directly acylated through the side chain amine, hydroxyl, or thiol of an amino acid. In some embodiments, acylation is at a position corresponding to A10, B28, B29 of native insulin, or at the C-terminus or N-terminus of the A or B chain. In this regard, the acylated IGF^{B16B17} derivative peptide can comprise the amino acid sequence of SEQ ID NO: 9 and SEQ ID NO: 10, or a modified amino acid sequence thereof comprising one or more of the amino acid modifications described herein, with at least one of the amino acids at a position corresponding to A10, B28, B29 of native insulin, or at the C-terminus or N-terminus of the A or B chain modified to any amino acid comprising a side chain amine, hydroxyl, or thiol. In some specific embodiments, the direct acylation of the IGF^{B16B17} derivative peptide occurs through the side chain amine, hydroxyl, or thiol of the amino acid at a position corresponding to A10 or B29 of native insulin.

In some embodiments, the amino acid comprising a side chain amine is an amino acid of Formula VI:

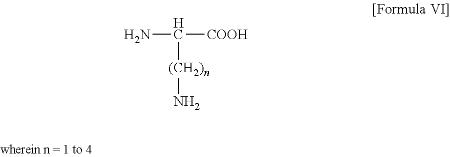

In some exemplary embodiments, the amino acid of Formula VI, is the amino acid wherein n is 4 (Lys) or n is 3 (Orn).

In other embodiments, the amino acid comprising a side chain hydroxyl is an amino acid of Formula IV:

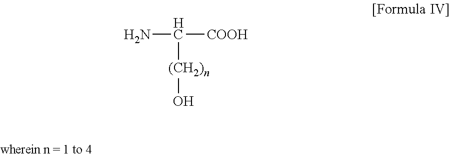

In some exemplary embodiments, the amino acid of Formula IV is the amino acid wherein n is 1 (Ser).

In yet other embodiments, the amino acid comprising a side chain thiol is an amino acid of Formula V:

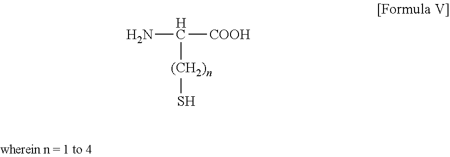

In some exemplary embodiments, the amino acid of Formula V is the amino acid wherein n is 1 (Cys).

In some exemplary embodiments, the IGF^{B16B17} derivative peptide is modified to comprise an acyl group by acylation of an amine, hydroxyl, or thiol of a spacer, which spacer is attached to a side chain of an amino acid at position A10, B28 or B29 (according to the amino acid numbering of wild type insulin). The amino acid to which the spacer is attached can be any amino acid comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol, or a dipeptide or tripeptide comprising an amino acid comprising a side chain amine, hydroxyl, or thiol.

When acylation occurs through an amine group of a spacer the acylation can occur through the alpha amine of the amino acid or a side chain amine. In the instance in which the alpha amine is acylated, the spacer amino acid can be any amino acid. For example, the spacer amino acid can be a hydrophobic amino acid, e.g., Gly, Ala, Val, Leu, Ile, Trp, Met, Phe, Tyr. Alternatively, the spacer amino acid can be an acidic residue, e.g., Asp and Glu. In the instance in which the side chain amine of the spacer amino acid is acylated, the spacer amino acid is an amino acid comprising a side chain amine, e.g., an amino acid of Formula IV (e.g., Lys or Orn). In this instance, it is possible for both the alpha amine and the side chain amine of the spacer amino acid to be acylated, such that the IGF^{B16B17} derivative peptide is diacylated. The present disclosure further contemplates diacylated IGF^{B16B17} derivative peptides.

When acylation occurs through a hydroxyl group of a spacer, the amino acid or one of the amino acids of the dipeptide or tripeptide can be an amino acid of Formula V. In a specific exemplary embodiment, the amino acid is Ser.

When acylation occurs through a thiol group of a spacer, the amino acid or one of the amino acids of the dipeptide or tripeptide can be an amino acid of Formula V. In a specific exemplary embodiment, the amino acid is Cys.

In one embodiment, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.).

Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmacuetical Res. “Synthesis of Palmitoyl Derivatives of Insulin and their Biological Activity” Vol. 6, No: 2 pp. 171-176 (1989).

The acyl group of the acylated IGF^{B16B17} derivative peptide can be of any size, e.g., any length carbon chain, and can be linear or branched. In some specific embodiments of the invention, the acyl group is a C4 to C30 fatty acid. For example, the acyl group can be any of a C4 fatty acid, C6 fatty acid, C8 fatty acid, C10 fatty acid, C12 fatty acid, C14 fatty acid, C16 fatty acid, C18 fatty acid, C20 fatty acid,

C22 fatty acid, C24 fatty acid, C26 fatty acid, C28 fatty acid, or a C30 fatty acid. In some embodiments, the acyl group is a C8 to C20 fatty acid, e.g., a C14 fatty acid or a C16 fatty acid.

In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid.

In a specific embodiment, the IGF^{B16B17} derivative peptide comprises a cholesterol acid, which is linked to a Lys residue of the IGF^{B16B17} derivative peptide through an alkylated des-amino Cys spacer, i.e., an alkylated 3-mercaptopropionic acid spacer. The alkylated des-amino Cys spacer can be, for example, a des-amino-Cys spacer comprising a dodecaethylene glycol moiety. In one embodiment, the IGF^{B16B17} derivative peptide comprises the structure:

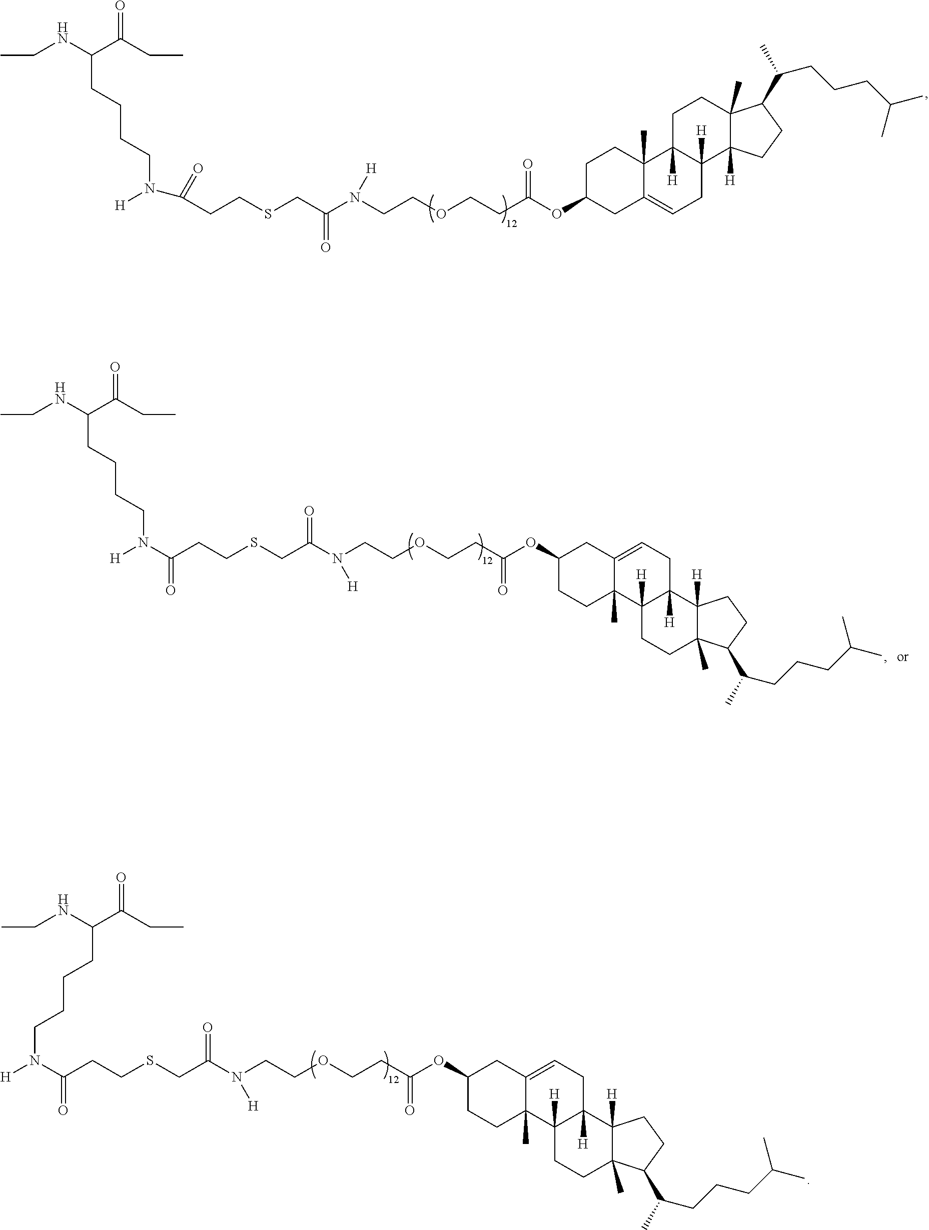

The acylated IGF^{B16B17} derivative peptides described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein.

Alternatively, the acylated IGF^{B16B17} derivative peptide can comprise a spacer, wherein the spacer is both acylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In accordance with one embodiment, the IGF^{B16B17} derivative peptide is modified to comprise an alkyl group which is attached to the IGF^{B16B17} derivative peptide via an ester, ether, thioether, amide, or alkyl amine linkage for purposes of prolonging half-life in circulation and/or delaying the onset of and/or extending the duration of action and/or improving resistance to proteases such as DPP-IV.

The alkyl group of the alkylated IGF^{B16B17} derivative peptide can be of any size, e.g., any length carbon chain, and can be linear or branched. In some embodiments of the invention, the alkyl group is a C1 to C30 alkyl. For example, the alkyl group can be any of a C1 alkyl, C2 alkyl, C3 alkyl, C4 alkyl, C6 alkyl, C8 alkyl, C10 alkyl, C12 alkyl, C14 alkyl, C16 alkyl, C18 alkyl, C20 alkyl, C22 alkyl, C24 alkyl, C26 alkyl, C28 alkyl, or a C30 alkyl. In some embodiments, the alkyl group is a C8 to C20 alkyl, e.g., a C14 alkyl or a C16 alkyl.

In some specific embodiments, the alkyl group comprises a steroid moiety of a bile acid, e.g., cholic acid, chenodeoxycholic acid, deoxycholic acid, lithocholic acid, taurocholic acid, glycocholic acid, and cholesterol acid.

In accordance with one embodiment a pharmaceutical composition is provided comprising any of the novel IGF^{B16B17} derivative peptides disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain an IGF^{B16B17} derivative peptide as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored contained within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

In one embodiment, a composition is provided comprising a mixture of a first and second IGF^{B16B17} derivative peptide prodrug analog, wherein the first and second IGF^{B16B17} derivative peptide prodrug analogs differ from one another based on the structure of the prodrug element. More particularly, the first IGF^{B16B17} derivative peptide prodrug analog may comprise a dipeptide prodrug element that has a half life substantially different from the dipeptide prodrug element of the second IGF^{B16B17} derivative peptide prodrug analog. Accordingly, selection of different combinations of substituents on the dipeptide element will allow for the preparation of compositions that comprise a mixture of IGF^{B16B17} derivative peptide prodrug analogs that are activated in a controlled manner over a desired time frame and at specific time intervals. For example, the compositions can be formulated to release active IGF^{B16B17} derivative peptide at mealtimes followed by a subsequent activation of IGF^{B16B17} derivative peptide during nighttime with suitable dosages being released based on time of activation. In another embodiment the pharmaceutical composition comprises a mixture of an IGF^{B16B17} derivative peptide prodrug analog disclosed herein and native insulin, or a known bioactive derivative of insulin. The mixture in one embodiment can be in the form of a heterodimer linking an IGF^{B16B17} derivative peptide analog and a native insulin, or a known bioactive derivative of insulin. The dimers may comprise single chain insulin/IGF derivative peptide or disulfide linked A chain to B chain heterodimers. The mixtures may comprise one or more IGF^{B16B17} derivative peptide analogs, native insulin, or a known bioactive derivative of insulin in prodrug forms, depot derivative or other conjugate forms, and any combination thereof, as disclosed herein.

The disclosed IGF^{B16B17} derivative peptides, and their corresponding prodrug derivatives, are believed to be suitable for any use that has previously been described for insulin peptides. Accordingly, the IGF^{B16B17} derivative peptides, and their corresponding prodrug derivatives, described herein can be used to treat hyperglycemia, or treat other metabolic diseases that result from high blood glucose levels. Accordingly, the present invention encompasses pharmaceutical compositions comprising an IGF^{B16B17} derivative peptide of the present disclosure, or a prodrug derivative thereof, and a pharmaceutically acceptable carrier for use in treating a patient suffering from high blood glucose levels. In accordance with one embodiment the patient to be treated using the IGF^{B16B17} derivative peptides disclosed herein is a domesticated animal, and in another embodiment the patient to be treated is a human.

One method of treating hyperglycemia in accordance with the present disclosure comprises the steps of administering the presently disclosed IGF^{B16B17} derivative peptide, or depot or prodrug derivative thereof, to a patient using any standard route of administration, including parenterally, such as intravenously, intraperitoneally, subcutaneously or intramuscularly, intrathecally, transdermally, rectally, orally, nasally or by inhalation. In one embodiment the composition is administered subcutaneously or intramuscularly. In one embodiment, the composition is administered parenterally and the IGF^{B16B17} derivative peptide, or prodrug derivative thereof, composition is prepackaged in a syringe.

The IGF^{B16B17} derivative peptides disclosed herein, and depot or prodrug derivative thereof, may be administered alone or in combination with other anti-diabetic agents. Anti-diabetic agents known in the art or under investigation include native insulin, native glucagon and functional derivatives thereof, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; or FBPase (fructose 1,6-bisphosphatase) inhibitors.

Pharmaceutical compositions comprising the IGF^{B16B17} derivative peptides disclosed herein, or depot or prodrug derivatives thereof, can be formulated and administered to patients using standard pharmaceutically acceptable carriers and routes of administration known to those skilled in the art. Accordingly, the present disclosure also encompasses pharmaceutical compositions comprising one or more of the IGF^{B16B17} derivative peptides disclosed herein (or prodrug derivative thereof), or a pharmaceutically acceptable salt thereof, in combination with a pharmaceutically acceptable carrier. In one embodiment the pharmaceutical composition comprises a 1 mg/ml concentration of the IGF^{B16B17} derivative peptide at pH of about 4.0 to about 7.0 in a phosphate buffer system. The pharmaceutical compositions may comprise the IGF^{B16B17} derivative peptide as the sole pharmaceutically active component, or the IGF^{B16B17} derivative peptide can be combined with one or more additional active agents. In accordance with one embodiment a pharmaceutical composition is provided comprising one of the IGF^{B16B17} derivative peptides disclosed herein (or depot or prodrug derivative thereof), preferably sterile and preferably at a purity level of at least 90%, 91%, 92%, 93%, 94%, 95%, 96%, 97%, 98% or 99%, and a pharmaceutically acceptable diluent, carrier or excipient. Such compositions may contain an IGF^{B16B17} derivative peptide wherein the resulting active peptide is present at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored within various containers. The compounds of the present invention can be used in accordance with one embodiment to prepare pre-formulated solutions ready for injection. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

All therapeutic methods, pharmaceutical compositions, kits and other similar embodiments described herein contemplate that IGF^{B16B17} derivative peptides, or prodrug derivatives thereof, include all pharmaceutically acceptable salts thereof.

In one embodiment the kit is provided with a device for administering the IGF^{B16B17} derivative peptide composition to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the IGF^{B16B17} derivative peptide composition is prepackaged within the syringe.

The compounds of this invention may be prepared by standard synthetic methods, recombinant DNA techniques, or any other methods of preparing peptides and fusion proteins. Although certain non-natural amino acids cannot be expressed by standard recombinant DNA techniques, techniques for their preparation are known in the art. Compounds of this invention that encompass non-peptide portions may be synthesized by standard organic chemistry reactions, in addition to standard peptide chemistry reactions when applicable.

### EXAMPLE 1

Synthesis of Insulin A & B Chains

Insulin A & B chains were synthesized on 4-methylbenzhyryl amine (MBHA) resin or 4-Hydroxymethyl-phenylacetamidomethyl (PAM) resin using Boc chemistry. The peptides were cleaved from the resin using HF/p-cresol 95:5 for 1 hour at 0° C. Following HF removal and ether precipitation, peptides were dissolved into 50% aqueous acetic acid and lyophilized. Alternatively, peptides were synthesized using Fmoc chemistry. The peptides were cleaved from the resin using Trifluoroacetic acid (TFA)/Triisopropylsilane (TIS)/H₂O (95:2.5:2.5), for 2 hour at room temperature. The peptide was precipitated through the addition of an excessive amount of diethyl ether and the pellet solubilized in aqueous acidic buffer. The quality of peptides were monitored by RP-HPLC and confirmed by Mass Spectrometry (ESI or MALDI).

Insulin A chains were synthesized with a single free cysteine at amino acid 7 and all other cysteines protected as acetamidomethyl A-(SH)⁷(Acm)^{6,11,20} Insulin B chains were synthesized with a single free cysteine at position 7 and the other cysteine protected as acetamidomethyl B-(SH)⁷(Acm)¹⁹. The crude peptides were purified by conventional RP-HPLC.

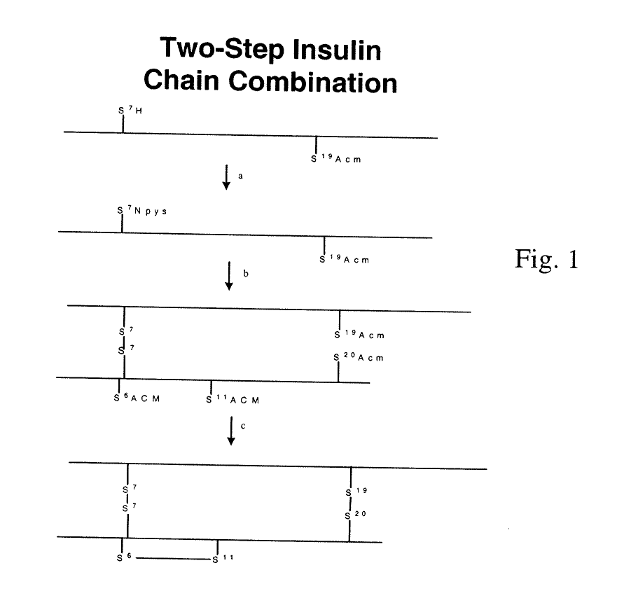

The synthesized A and B chains were linked to one another through their native disulfide bond linkage in accordance with the general procedure outlined in FIG. 1. The respective B chain was activated to the Cys⁷-Npys derivative through dissolution in DMF or DMSO and reacted with 2,2′-Dithiobis (5-nitropyridine) (Npys) at a 1:1 molar ratio, at room temperature. The activation was monitored by RP-HPLC and the product was confirmed by ESI-MS.

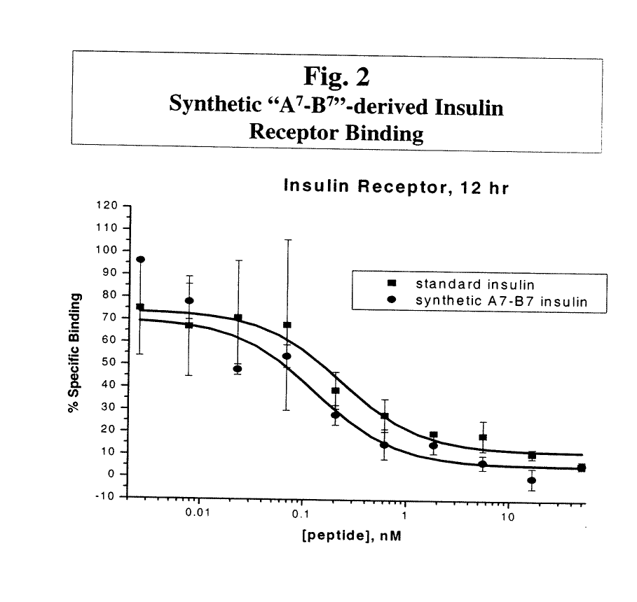

The first B7-A7 disulfide bond was formed by dissolution of the respective A-(SH)⁷(Acm)^{6,11,20} and B-(Npys)⁷(Acm)¹⁹ at 1:1 molar ratio to a total peptide concentration of 10 mg/ml. When the chain combination reaction was complete the mixture was diluted to a concentration of 50% aqueous acetic acid. The last two disulfide bonds were formed simultaneously through the addition of iodine. A 40 fold molar excess of iodine was added to the solution and the mixture was stirred at room temperature for an additional hour. The reaction was terminated by the addition of an aqueous ascorbic acid solution. The mixture was purified by RP-HPLC and the final compound was confirmed by MALDI-MS. As shown in FIG. 2 and the data in Table 1, the synthetic insulin prepared in accordance with this procedure compares well with purified insulin for insulin receptor binding.

Insulin peptides comprising a modified amino acid (such as 4-amino phenylalanine at position A19) can also be synthesized in vivo using a system that allows for incorporation of non-coded amino acids into proteins, including for example, the system taught in U.S. Pat. Nos. 7,045,337 and 7,083,970.

TABLE 1
Activity of synthesized insulin relative to native insulin

| | Insulin | | | |
|---|---|---|---|---|
| | Standard | | A7-B7 Insulin | |
| | AVER. | STDEV | AVER. | STDEV |
| IC₅₀ (nM) | 0.24 | 0.07 | 0.13 | 0.08 |
| % of Insulin Activity | 100 | | 176.9 | |

### EXAMPLE 2

### Pegylation of Amine Groups (N-Terminus and Lysine) by Reductive Alkylation

a. Synthesis

Insulin (or an insulin analog), mPEG20k-Aldyhyde, and NaBH₃CN, in a molar ratio of 1:2:30, were dissolved in acetic acid buffer at a pH of 4.1-4.4. The reaction solution was composed of 0.1 N NaCl, 0.2 N acetic acid and 0.1 N Na₂CO₃. The insulin peptide concentration was approximately 0.5 mg/ml. The reaction occurs over six hours at room temperature. The degree of reaction was monitored by RP-HPLC and the yield of the reaction was approximately 50%.

b. Purification

The reaction mixture was diluted 2-5 fold with 0.1% TFA and applied to a preparative RP-HPLC column. HPLC condition: C4 column; flow rate 10 ml/min; A buffer 10% ACN and 0.1% TFA in water; B buffer 0.1% TFA in ACN; A linear gradient B% from 0-40% (0-80 min); PEG-insulin or analogues was eluted at approximately 35% buffer B. The desired compounds were verified by MALDI-TOF, following chemical modification through sulftolysis or trypsin degradation.

### Pegylation of Amine Groups (N-Terminus and Lysine) by N-Hydroxysuccinimide Acylation.

a. Synthesis

Insulin (or an insulin analog) along with mPEG20k-NHS were dissolved in 0.1 N Bicine buffer (pH 8.0) at a molar ratio of 1:1. The insulin peptide concentration was approximately 0.5 mg/ml. Reaction progress was monitored by HPLC. The yield of the reaction is approximately 90% after 2 hours at room temperature.

b. Purification

The reaction mixture was diluted 2-5 fold and loaded to RP-HPLC. HPLC condition: C4 column; flow rate 10 ml/min; A buffer 10% ACN and 0.1% TFA in water; B buffer 0.1% TFA in ACN; A linear gradient B% from 0-40% (0-80 min); PEG-insulin or analogues was collected at approximately 35% B. The desired compounds were verified by MALDI-TOF, following chemical modification through sulftolysis or trypsin degradation.

### Reductive Aminated Pegylation of Acetyl Group on the Aromatic Ring of the Phenylalanine

a. Synthesis

Insulin (or an insulin analogue), mPEG20k-Hydrazide, and NaBH₃CN in a molar ratio of 1:2:20 were dissolved in acetic acid buffer (pH of 4.1 to 4.4). The reaction solution was composed of 0.1 N NaCl, 0.2 N acetic acid and 0.1 N Na₂CO₃. Insulin or insulin analogue concentration was approximately 0.5 mg/ml. at room temperature for 24 h. The reaction process was monitored by HPLC. The conversion of the reaction was approximately 50%. (calculated by HPLC)

b. Purification

The reaction mixture was diluted 2-5 fold and loaded to RP-HPLC. HPLC condition: C4 column; flow rate 10 ml/min; A buffer 10% ACN and 0.1% TFA in water; B buffer 0.1% TFA in ACN; A linear gradient B% from 0-40% (0-80 min); PEG-insulin, or the PEG-insulin analogue was collected at approximately 35%B. The desired compounds were verified by MALDI-TOF, following chemical modification through sulftolysis or trypsin degradation.

### EXAMPLE 3

### Insulin Receptor Binding Assay:

The affinity of each peptide for the insulin or IGF-1 receptor was measured in a competition binding assay utilizing scintillation proximity technology. Serial 3-fold dilutions of the peptides were made in Tris-Cl buffer (0.05 M Tris-HCl, pH 7.5, 0.15 M NaCl, 0.1% w/v bovine serum albumin) and mixed in 96 well plates (Corning Inc., Acton, Mass.) with 0.05 nM (3-[1251]-iodotyrosyl) A TyrA14 insulin or (3-[1251]-iodotyrosyl) IGF-1 (Amersham Biosciences, Piscataway, N.J.). An aliquot of 1-6 micrograms of plasma membrane fragments prepared from cells over-expressing the human insulin or IGF-1 receptors were present in each well and 0.25 mg/well polyethylene imine-treated wheat germ agglutinin type A scintillation proximity assay beads (Amersham Biosciences, Piscataway, N.J.) were added. After five minutes of shaking at 800 rpm the plate was incubated for 12 h at room temperature and radioactivity was measured with MicroBeta1450 liquid scintillation counter (Perkin-Elmer, Wellesley, Mass.). Non-specifically bound (NSB) radioactivity was measured in the wells with a four-fold concentration excess of “cold” native ligand than the highest concentration in test samples. Total bound radioactivity was detected in the wells with no competitor. Percent specific binding was calculated as following: % Specific Binding=(Bound−NSB/Total bound−NSB)×100. IC50 values were determined by using Origin software (OriginLab, Northampton, Mass.).

### EXAMPLE 4

### Insulin Receptor Phosphorylation Assay:

To measure receptor phosphorylation of insulin or insulin analog, receptor transfected HEK293 cells were plated in 96 well tissue culture plates (Costar #3596, Cambridge, Mass.) and cultured in Dulbecco's modified Eagle medium (DMEM) supplemented with 100 IU/ml penicillin, 100 μg/ml streptomycin, 10 mM HEPES and 0.25% bovine growth serum (HyClone SH30541, Logan, Utah) for 16-20 hrs at 37° C., 5% CO₂ and 90% humidity. Serial dilutions of insulin or insulin analogs were prepared in DMEM supplemented with 0.5% bovine serum albumin (Roche Applied Science #100350, Indianapolis, Ind.) and added to the wells with adhered cells. After 15 min incubation at 37° C. in humidified atmosphere with 5% CO₂ the cells were fixed with 5% paraformaldehyde for 20 min at room temperature, washed twice with phosphate buffered saline pH 7.4 and blocked with 2% bovine serum albumin in PBS for 1 hr. The plate was then washed three times and filled with horseradish peroxidase-conjugated antibody against phosphotyrosine (Upstate biotechnology #16-105, Temecula, Calif.) reconstituted in PBS with 2% bovine serum albumin per manufacturer's recommendation. After 3 hrs incubation at room temperature the plate was washed 4 times and 0.1 ml of TMB single solution substrate (Invitrogen, #00-2023, Carlbad, Calif.) was added to each well. Color development was stopped 5 min later by adding 0.05 ml 1 N HCl. Absorbance at 450 nm was measured on Titertek Multiscan MCC340 (ThermoFisher, Pittsburgh, Pa.). Absorbance vs. peptide concentration dose response curves were plotted and EC₅₀ values were determined by using Origin software (OriginLab, Northampton, Mass.).

### EXAMPLE 5

### Determination of Rate of Model Dipeptide Cleavage (in PBS)

A specific hexapeptide (HSRGTF-NH₂; SEQ ID NO: 73) was used as a model peptide upon which the rate of cleavage of dipeptide N-terminal extensions could be studied. The dipeptide-extended model peptides were prepared Boc-protected sarcosine and lysine were successively added to the model peptide-bound resin to produce peptide A (Lys-Sar-HSRGTF-NH₂; SEQ ID NO: 74). Peptide A was cleaved by HF and purified by preparative HPLC.

Preparative Purification Using HPLC:

Purification was performed using HPLC analysis on a silica based 1×25 cm Vydac C18 (5μ particle size, 300 A° pore size) column. The instruments used were: Waters Associates model 600 pump, Injector model 717, and UV detector model 486. A wavelength of 230 nm was used for all samples. Solvent A contained 10% CH₃CN/0.1% TFA in distilled water, and solvent B contained 0.1% TFA in CH₃CN. A linear gradient was employed (0 to 100% B in 2 hours). The flow rate was 10 ml/min and the fraction size was 4 ml. From ˜150 mgs of crude peptide, 30 mgs of the pure peptide was obtained. Peptide A was dissolved at a concentration of 1 mg/ml in PBS buffer. The solution was incubated at 37° C. Samples were collected for analysis at 5 h, 8 h, 24 h, 31 h, and 47 h. The dipeptide cleavage was quenched by lowering the pH with an equal volume of 0.1% TFA. The rate of cleavage was qualitatively monitored by LC-MS and quantitatively studied by HPLC. The retention time and relative peak area for the prodrug and the parent model peptide were quantified using Peak Simple Chromatography software.

### Analysis Using Mass Spectrometry

The mass spectra were obtained using a Sciex API-III electrospray quadrapole mass spectrometer with a standard ESI ion source. Ionization conditions that were used are as follows: ESI in the positive-ion mode; ion spray voltage, 3.9 kV; orifice potential, 60 V. The nebulizing and curtain gas used was nitrogen flow rate of 0.9 L/min. Mass spectra were recorded from 600-1800 Thompsons at 0.5 Th per step and 2 msec dwell time. The sample (about 1 mg/mL) was dissolved in 50% aqueous acetonitrile with 1% acetic acid and introduced by an external syringe pump at the rate of 5 μL/min. Peptides solubilized in PBS were desalted using a ZipTip solid phase extraction tip containing 0.6 μL C4 resin, according to instructions provided by the manufacturer (Millipore Corporation, Billerica, Mass.) prior to analysis.

### Analysis Using HPLC

The HPLC analyses were performed using a Beckman System Gold Chromatography system equipped with a UV detector at 214 nm and a 150 mm×4.6 mm C8 Vydac column. The flow rate was 1 ml/min. Solvent A contained 0.1% TFA in distilled water, and solvent B contained 0.1% TFA in 90% CH₃CN. A linear gradient was employed (0% to 30% B in 10 minutes). The data were collected and analyzed using Peak Simple Chromatography software.

The rate of cleavage was determined for the respective propeptides. The concentrations of the propeptides and the model parent peptide were determined by their respective peak areas. The first order dissociation rate constants of the prodrugs were determined by plotting the logarithm of the concentration of the prodrug at various time intervals. The slope of this plot provides the rate constant ‘k’. The half lives for cleavage of the various prodrugs were calculated by using the formula t_{1/2}=0.693/k. The half life of the Lys-Sar extension to this model peptide HSRGTF—NH₂ (SEQ ID NO: 73) was determined to be 14.0 h.

### EXAMPLE 6

Rate of Dipeptide Cleavage Half Time in Plasma as Determined with an All d-Isoform Model Peptide

An additional model hexapeptide (dHdTdRGdTdF—NH₂ SEQ ID NO: 75) was used to determine the rate of dipeptide cleavage in plasma. The d-isomer of each amino acid was used to prevent enzymatic cleavage of the model peptide, with the exception of the prodrug extension. This model d-isomer hexapeptide was synthesized in an analogous fashion to the 1-isomer. The sarcosine and lysine were successively added to the N-terminus as reported previously for peptide A to prepare peptide B (dLys-dSar-dHdTdRGdTdF—NH₂ SEQIDNO: 76)

The rate of cleavage was determined for the respective propeptides. The concentrations of the propeptides and the model parent peptide were determined by their respective peak areas. The first order dissociation rate constants of the prodrugs were determined by plotting the logarithm of the concentration of the prodrug at various time intervals. The slope of this plot provides the rate constant ‘k’. The half life of the Lys-Sar extension to this model peptide dHdTdRGdTdF—NH₂ (SEQ ID NO: 75) was determined to be 18.6 h.

### EXAMPLE 7

The rate of cleavage for additional dipeptides linked to the model hexapeptide (HSRGTF-NH₂; SEQ ID NO: 77) were determined using the procedures described in Example 5. The results generated in these experiments are presented in Tables 2 and 3.

TABLE 2
Cleavage of the Dipeptide O—U that are linked to the side chain of an N-
terminal para-amino-Phe from the Model Hexapeptide (HSRGTF-NH₂;
SEQ ID NO: 59) in PBS

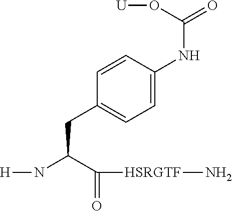

| Com- | U | O (amino | |
|---|---|---|---|
| pounds | (amino acid) | acid) | t½ |
| 1 | F | P | 58 h |
| 2 | Hydroxyl-F | P | 327 h |
| 3 | d-F | P | 20 h |
| 4 | d-F | d-P | 39 h |
| 5 | G | P | 72 h |
| 6 | Hydroxyl-G | P | 603 h |
| 7 | L | P | 62 h |
| 8 | tert-L | P | 200 h |
| 9 | S | P | 34 h |
| 10 | P | P | 97 h |
| 11 | K | P | 33 h |
| 12 | dK | P | 11 h |
| 13 | E | P | 85 h |
| 14 | Sar | P | ≈1000 h |
| 15 | Aib | P | 69 min |
| 16 | Hydroxyl- | P | 33 h |
| | Aib | | |
| 17 | cyclohexane | P | 6 min |
| 18 | G | G | No cleavage |
| 19 | Hydroxyl-G | G | No cleavage |
| 20 | S | N-Methyl- | 4.3 h |
| | | Gly | |
| 21 | K | N-Methyl- | 5.2 h |
| | | Gly | |
| 22 | Aib | N-Methyl- | 7.1 min |
| | | Gly | |
| 23 | Hydroxyl-Aib | N-Methyl- | 1.0 h |
| | | Gly | |

TABLE 3
Cleavage of the Dipeptides U-O linked to histidine
(or histidine derivative) at position 1 (X) from the Model Hexapeptide
(XSRGTF-NH₂; SEQ ID NO: 59) in PBS
NH₂-U-O-XSRGTF-NH₂

| Comd. | U (amino acid) | O (amino acid) | X (amino acid) | t_{1/2} | |
|---|---|---|---|---|---|
| 1 | F | P | H | No cleavage | |
| 2 | Hydroxyl-F | P | H | No cleavage | |
| 3 | G | P | H | No cleavage | |
| 4 | Hydroxyl-G | P | H | No cleavage | |
| 5 | A | P | H | No cleavage | |
| 6 | C | P | H | No cleavage | |
| 7 | S | P | H | No cleavage | |
| 8 | P | P | H | No cleavage | |
| 9 | K | P | H | No cleavage | |
| 10 | E | P | H | No cleavage | |
| 11 | Dehydro V | P | H | No cleavage | |
| 12 | P | d-P | H | No cleavage | |
| 13 | d-P | P | H | No cleavage | |
| 14 | Aib | P | H | 32 | h |
| 15 | Aib | d-P | H | 20 | h |
| 16 | Aib | P | d-H | 16 | h |
| 17 | Cyclohexyl- | P | H | 5 | h |
| 18 | Cyclopropyl- | P | H | 10 | h |
| 19 | N-Me-Aib | P | H | >500 | h |
| 20 | α, | P | H | 46 | h |
| | α-diethyl-Gly | | | | |
| 21 | Hydroxyl-Aib | P | H | 61 | |
| 22 | Aib | P | A | 58 | |
| 23 | Aib | P | N-Methyl-His | 30 | h |
| 24 | Aib | N-Methyl-Gly | H | 49 | min |
| 25 | Aib | N-Hexyl-Gly | H | 10 | min |
| 26 | Aib | Azetidine-2- | H | >500 | h |
| | | carboxylic acid | | | |
| 27 | G | N-Methyl-Gly | H | 104 | h |
| 28 | Hydroxyl-G | N-Methyl-Gly | H | 149 | h |
| 29 | G | N-Hexyl-Gly | H | 70 | h |
| 30 | dK | N-Methyl-Gly | H | 27 | h |
| 31 | dK | N-Methyl-Ala | H | 14 | h |
| 32 | dK | N-Methyl-Phe | H | 57 | h |
| 33 | K | N-Methyl-Gly | H | 14 | h |
| 34 | F | N-Methyl-Gly | H | 29 | h |
| 35 | S | N-Methyl-Gly | H | 17 | h |
| 36 | P | N-Methyl-Gly | H | 181 | h |

### EXAMPLE 8

Identification of an Insulin Analog with Structure Suitable for Prodrug Construction

Position 19 of the A chain is known to be an important site for insulin activity. Modification at this site to allow the attachment of a prodrug element is therefore desirable. Specific analogs of insulin at A19 have been synthesized and characterized for their activity at the insulin receptors. Two highly active structural analogs have been identified at A19, wherein comparable structural changes at a second active site aromatic residue (B24) were not successful in identification of similarly full activity insulin analogs.

Tables 4 and 5 illustrate the high structural conservation at position A19 for full activity at the insulin receptor (receptor binding determined using the assay described in Example 3). Table 4 demonstrates that only two insulin analogs with modifications at A19 have receptor binding activities similar to native insulin. For the 4-amino insulin analog, data from three separate experiments is provided. The column labeled “Activity (in test)” compares the percent binding of the insulin analog relative to native insulin for two separate experiments conducted simultaneously. The column labeled “Activity (0.60 nM)” is the relative percent binding of the insulin analog relative to the historical average value obtained for insulin binding using this assay. Under either analysis, two A19 insulin analogs (4-amino phenylalanine and 4-methoxy phenylalanine) demonstrate receptor binding approximately equivalent to native insulin. FIG. 3 represents a graph demonstrating the respective specific binding of native insulin and the A19 insulin analog to the insulin receptor. Table 5 presents data showing that the two A19 insulin analogs (4-amino and 4-methoxy) that demonstrate equivalent binding activities as native insulin also demonstrate equivalent activity at the insulin receptor (receptor activity determined using the assay described in Example 4).

TABLE 4
Insulin Receptor Binding Activity of A19 Insulin Analogs

| | Insulin Receptor | | | |
|---|---|---|---|---|
| | | | % native | % native |
| | | | ligand | ligand |
| | | | Activity | Activity |
| Analogue | IC₅₀ | STDev | (in test) | (0.60 nM) |
| 4-OH (native insulin) | 0.64 | 0.15 | 100.0 | 100.0 |
| 4-COCH₃ | 31.9 | 9.47 | 0.6 | 1.9 |
| 4-NH₂ | 0.31 | 0.12 | 203.0 | 193.5 |
| | 0.83 | 0.15 | 103.0 | 72.3 |
| | 0.8 | 0.1 | 94.0 | 75.0 |
| 4-NO₂ | 215.7 | 108.01 | 0.3 | 1.3 |
| 3,4,5-3F | 123.29 | 31.10 | 0.5 | 0.5 |
| 4-OCH₃ | 0.5 | 0.50 | 173.0 | 120.0 |
| 3-OCH₃ | 4.74 | 1.09 | 28.0 | 12.7 |
| | 5.16 | 3.88 | 18.0 | 11.6 |
| 4-OH, 3,5-2Br | 1807.17 | 849.72 | 0.0 | 0.0 |
| 4-OH, 3,5-2NO₂ | 2346.2 | 338.93 | 0.0 | 0.0 |

TABLE 5
Insulin Receptor Phosphorylation Activity of A19 Insulin Analogs

| | Insulin Receptor | | |
|---|---|---|---|
| | | | % native ligand |
| Analogue | EC₅₀ | STDev | Activity (in test) |
| 4-OH (native insulin) | 1.22 | 0.4 | 100.0 |
| 4-NH₂ | 0.31 | 0.14 | 393.5 |
| 4-OCH₃ | 0.94 | 0.34 | 129.8 |

### EXAMPLE 9

Insulin like Growth Factor (IGF) Analog IGF1 (Y^{B16}L^{B17})

Applicants have discovered an IGF analog that demonstrates similar activity at the insulin receptor as native insulin. More particularly, the IGF analog (IGF1 (Y^{B16}L^{B17}) comprises the native IGF A chain (SEQ ID NO: 5) and the modified B chain (SEQ ID NO: 11), wherein the native glutamine and phenylalanine at positions 15 and 16 of the native IGF B-chain (SEQ ID NO: 6) have been replaced with tyrosine and leucine residues, respectively. As shown in FIG. 4 and Table 6 below the binding activities of IGF1 (Y^{B16}L^{B17}) and native insulin demonstrate that each are highly potent agonists of the insulin receptor.

TABLE 6

| | Insulin Standard | | IGF1(Y^{B16}L^{B17}) | |
|---|---|---|---|---|
| | AVER. | STDEV | AVER. | STDEV |
| IC₅₀ (nM) | 1.32 | 0.19 | 0.51 | 0.18 |
| % of Insulin Activity | 100 | | 262 | |

### EXAMPLE 10

### IGF Prodrug Derivatives

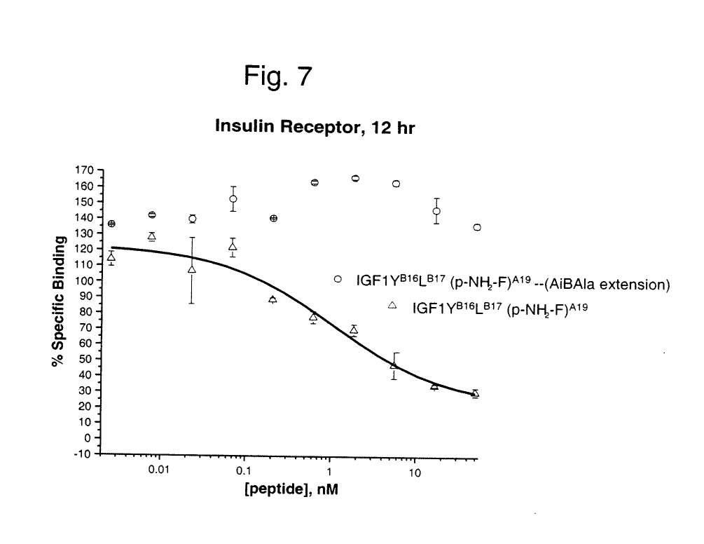

Based on the activity of the A19 insulin analog (see Example 5), a similar modification was made to the IGF1 A:B(Y^{B16}L^{B17}) analog and its ability to bind and stimulate insulin receptor activity was investigated. FIG. 6 provides the general synthetic scheme for preparing IGF1 A:B(Y^{B16}L^{B17}) wherein the native tyrosine is replace with a 4-amino phenylalanine [IGF1 A:B(Y^{B16}L¹⁷)(p-NH₂—F)^{A19}amide] as well as the preparation of its dipeptide extended derivative [IGF1 A:B(Y^{B16}L^{B17})^{A19}-AiBAla amide], wherein a dipeptide comprising AiB and Ala are linked to the peptide through an amide linkage to the A19 4-amino phenylalanine. As shown in FIG. 7 and Table 7, the IGF analog, IGF1 (Y^{B16}L^{B17}) A(p-NH₂—F)¹⁹ specifically binds to the insulin receptor wherein the dipeptide extended derivative of that analog fails to specifically bind the insulin receptor. Note the dipeptide extension lacks the proper structure to allow for spontaneous cleavage of the dipeptide (absence of an N-alkylated amino acid at the second position of the dipeptide) and therefore there is no restoration of insulin receptor binding.

IGF A:B(Y^{B16}L^{B17}) insulin analog peptides comprising a modified amino acid (such as 4-amino phenylalanine at position A19) can also be synthesized in vivo using a system that allows for incorporation of non-coded amino acids into proteins, including for example, the system taught in U.S. Pat. Nos. 7,045,337 and 7,083,970.

TABLE 7

| Insulin | | IGF1(Y^{B16}L^{B17}) | | IGF1(Y^{B16}L^{B17}) | |
|---|---|---|---|---|---|
| Standard | | (p-NH₂—F)^{A19}amide | | (AiBAla)^{A19}amide | |
| AVER. | STDEV | AVER. | STDEV. | AVER. | STDEV |

| | | | | | |
|---|---|---|---|---|---|
| IC₅₀ | 0.24 | 0.07 | 1.08 | .075 | No Activity |
| (nM) | | | | | |
| % of | 100 | | 22 | | |
| Insulin | | | | | |
| Activity | | | | | |

A further prodrug derivative of an IGF^{B16B17} derivative peptide was prepared wherein the dipeptide prodrug element (alanine-proline) was linked via an amide bond to the amino terminus of the A chain (IGF1(Y^{B16}L^{B17}) (AlaPro)^{A-1,0}). As shown in Table 8, the IGF1(Y^{B16}L^{B17})(AlaPro)^{A-1,0} has substantially reduced affinity for the insulin receptor. Note, based on the data of Table 3, the dipeptide prodrug element lacks the proper structure to allow for spontaneous cleavage of the dipeptide prodrug element, and therefore the detected insulin receptor binding is not the result of cleavage of the prodrug element.

TABLE 8

| | Insulin Standard | | IGF1(Y^{B16}L^{B17})(AlaPro)^{A-1,0} | |
|---|---|---|---|---|
| | AVER. | STDEV | AVER. | STDEV. |
| IC₅₀ (nM) | 0.72 | 0.09 | 1.93 | .96 |
| % of | 100 | | 37.12 | |
| Insulin | | | | |
| Activity | | | | |

### EXAMPLE 11

### Additional IGF Insulin Analogs.

Further modifications of the IGF1 (Y^{B16}L^{B17}) peptide sequence reveal additional IGF insulin analogs that vary in their potency at the insulin and IGF-1 receptor. Binding data is presented in Table 9 for each of these analogs (using the assay of Example 3), wherein the position of the modification is designated based on the corresponding position in the native insulin peptide (DPI=des B26-30). For example, a reference herein to “position B28” absent any further elaboration would mean the corresponding position B27 of the B chain of an insulin analog in which the first amino acid of SEQ ID NO: 2 has been deleted. Thus a generic reference to “B(Y16)” refers to a substitution of a tyrosine residue at position 15 of the B chain of the native IGF-1 sequence (SEQ ID NO: 6). Data regarding the relative receptor binding of insulin and IGF analogs is provided in Table 9, and data regarding IGF analog stimulated phosphorylation (using the assay of Example 4) is provided in Table 10.

TABLE 9
Receptor Binding Affinity of Insulin and IGF Analogues

| | Insulin Receptor | | | | | IGF-1 Receptor | | | | | |
|---|---|---|---|---|---|---|---|---|---|---|---|
| | | | | | % | | | | | % | |
| | | | | | native | | | | | native | |
| | | | | | insulin | | | | | IGF-1 | |
| | | | | % | activity | | | | % | activity | |
| | nM | | | insulin | (0.6 | | | | IGF-1 | (0.55 | |
| Analogue | IC₅₀: | STDev | Date | (in test) | nM) | IC₅₀: | STDev | Date | (in test) | nM) | Ratio |
| IGF-1 A:B | 10.41 | 1.65 | Sep. 4, 2007 | 5.8 | 5.8 | | | | | | |
| IGF-1 A:B(E10Y16L17) | 0.66 | 0.36 | May 22, 2007 | 58.7 | 90.9 | 7.85 | 1.98 | Jun. 4, 2007 | 6.8 | 7.0 | 11.9 |
| | 0.51 | 0.18 | May 29, 2007 | 98.8 | 117.6 | 12.19 | 2.17 | Sep. 18, 2007 | 5.0 | 4.5 | |
| IGF-1 A:B(E10 Y16L17)-E31E3 | 1.22 | 0.30 | Mar. 20, 2008 | 36.5 | 50.0 | 17.50 | 2.25 | Apr. 4, 2007 | 3.0 | 3.1 | 14.3 |
| 2B-COOH | | | | | | | | | | | |
| IGF-1 A:B(D10Y16L17) DPI A- | 0.26 | 0.02 | Nov. 9, 2007 | 301.0 | 231.0 | 6.79 | 1.50 | Apr. 4, 2008 | 7.7 | 8.1 | |
| COOH | | | | | | | | | | | |
| | 0.2 | 0.02 | Dec. 4, 2007 | 380.1 | 300.0 | | | | | | |
| | 0.42 | 0.06 | Jun. 5, 2008 | 174.1 | 144.1 | | | | | | |
| IGF-1 A:B (E10Y16L17) DPI | 0.38 | 0.08 | Aug. 10, 2007 | 51.1 | 157.9 | 22.89 | 5.26 | Sep. 18, 2007 | 3.3 | 2.4 | 60.2 |
| IGF-1 A:B (H5D10Y16L17) | 0.16 | 0.07 | Nov. 9, 2007 | 479.0 | | 4.66 | 0.77 | Apr. 4, 2008 | 11.2 | 11.8 | 29.1 |
| DPI | | | | | | | | | | | |
| IGF-1 A:B (H5D10Y16L17) | 0.25 | 0.04 | Nov. 9, 2007 | 316.0 | | | | | | | |
| (S═O)DPI | | | | | | | | | | | |
| IGF-1 A (H8 A9 N21): | 0.05 | 0.01 | Dec. 4, 2007 | 1576.7 | | 4.03 | 0.50 | Apr. 4, 2008 | 12.9 | 13.6 | 80.6 |
| B(H5D10Y16L17) | | | | | | | | | | | |
| DPI A-COOH | | | | | | | | | | | |
| | 0.09 | 0.02 | Dec. 14, 2007 | 1667.0 | | | | | | | |
| IGF-1 A (H8 A9 N21): | 0.12 | 0.02 | Dec. 14, 2007 | 1171.4 | | 22.83 | 3.53 | Apr. 4, 2008 | 2.3 | 2.4 | 190.3 |
| B(H5D10Y16L17 A22) DPI A- | | | | | | | | | | | |
| COOH | | | | | | | | | | | |
| IGF-1 A (H8 A9 N21): | 0.36 | 0.10 | Dec. 14, 2007 | 400.7 | | | | | | | |
| B(H5D10Y16L17A22) (S═O) | | | | | | | | | | | |
| DPI A-COOH | | | | | | | | | | | |
| IGF-1 A:IGF-1 B(1-8)-ln | 1.59 | 0.62 | May 22, 2007 | 19.1 | 37.7 | 131.30 | 58.05 | Jun. 4, 2007 | 0.3 | 0.4 | 82.6 |
| (9-17)-IGF-1 B(18-30) | | | | | | | | | | | |
| IGF-1 A:ln (1-17)-IGF-1 B (18- | 2.77 | 1.19 | May 22, 2007 | 14.0 | 21.7 | 62.50 | 30.28 | Jun. 4, 2007 | 0.9 | 0.9 | 22.6 |
| 30) | | | | | | | | | | | |
| | 2.67 | 0.67 | May 18, 2007 | 11.3 | 22.5 | | | | | | |
| | 2.48 | 1.35 | May 29, 2007 | 20.1 | 24.2 | | | | | | |
| IGF-1 A:ln B(1-5)-IGF-1 | 0.31 | 0.19 | Aug. 10, 2007 | 62.4 | 193.5 | 27.54 | 6.57 | Sep. 25, 2007 | 3.6 | 2 | 88.8 |
| B(YL)(6-30) | | | | | | | | | | | |
| IGF-2 native | | | | | | 13.33 | 1.85 | Sep. 25, 2007 | 7.5 | 4.5 | |
| IGF-2 AB | | | | | | | | | | | |
| IGF-2 AB(YL) | 6.81 | 3.81 | Oct. 10, 2007 | 8.4 | 8.8 | | | | | | |
| ln A:IGF-1 B(YL) | 82.62 | 31.75 | Sep. 4, 2007 | 0.9 | 0.7 | | | | | | |
| | 107.24 | 65.38 | Sep. 4, 2007 | 0.7 | 0.6 | | | | | | |
| ln A-IGF-2 D:ln B-IGF-2 C | 0.53 | 0.11 | Sep. 4, 2007 | 141.0 | 113.0 | 1.59 | 0.34 | Sep. 18, 2007 | 47.6 | 34.6 | |
| | 0.37 | 0.05 | Oct. 13, 2007 | 179.1 | 162.2 | 14.69 | 3.02 | Sep. 25, 2007 | 6.8 | 3.7 | 39.7 |

**All C terminals are amides (DPI) unless specified otherwise

TABLE 10
Total Phosphorylation by IGF-1 & IGF-2 Analogues

| | Insulin Receptor | | | | IGF-1 Receptor | | | | Selective |
|---|---|---|---|---|---|---|---|---|---|
| Analogue | EC50: | STDev | Date | % Insulin | EC50: | STDev | Date | % IGF | Ratio |
| Insulin | 1.26 | 0.098 | Dec. 14, 2007 | | 114.88 | 46.66 | Jan. 23, 2008 | | 90.89 |
| | 1.43 | 0.72 | Apr. 1, 2008 | | 86.02 | 29.35 | May 20, 2008 | | |
| | 1.12 | 0.11 | Mar. 31, 2008 | | | | | | |
| | 1.53 | 0.13 | Apr. 11, 2008 | | | | | | |
| | 2.70 | 0.71 | Apr. 16, 2008 | | | | | | |
| | 1.22 | 0.40 | May 20, 2008 | | | | | | |
| IGF-1 | 54.39 | 21.102 | Dec. 14, 2007 | 2.3 | 0.87 | 0.16 | Jan. 23, 2008 | 100 | 0.02 |
| | | | | | 0.49 | 0.13 | May 20, 2008 | | |
| | | | | | 0.97 | 0.48 | Jul. 23, 2008 | | |
| IGF-1 AB | | | | | | | | | |
| IGF-1 A:B(E10Y16L17) | 2.57 | 0.59 | Mar. 31, 2008 | 49.2 | 7.42 | 5.59 | Jul. 23, 2008 | 13 | |
| IGF-1 A:B(E10 Y16L17)-E31E32 | 7.00 | 2.82 | Mar. 31, 2008 | 18.1 | | | | | |
| B-COOH | | | | | | | | | |
| | 8.52 | 4.34 | Apr. 16, 2008 | 31.7 | | | | | |
| IGF-1 AB(D10Y16L17) DPI A-COOH | 0.08 | 0.006 | Dec. 14, 2007 | 1575 | 0.78 | 0.17 | Jan. 23, 2008 | 111.538 | 9.75 |
| | 4.38 | 2.98 | Apr. 16, 2008 | ?? | | | | | |
| IGF-1 AB (E10Y16L17) DPI | | | | | | | | | |
| IGF-1 AB (H5D10Y16L17) DPI | | | | | 12.22 | 5.46 | Jan. 23, 2008 | 7.1 | |
| IGF-1 AB (H5D10Y16L17) (S═O)DPI | | | | | | | | | |
| IGF-1 A (H8 A9 N21) B(H5D10Y16L17) | 0.15 | 0.054 | Dec. 14, 2007 | 840 | 0.43 | 0.44 | Jan. 23, 2008 | 181.395 | 2.81 |
| DPI A-COOH | | | | | | | | | |
| | 0.25 | 0.2 | Apr. 16, 2008 | 1080 | | | | | |
| IGF-1 A (H8 A9 N21) | 0.35 | 0.064 | Dec. 14, 2007 | 360 | 11.26 | 2.55 | Jan. 23, 2008 | 7.7 | 32.54 |
| B(H5D10Y16L17A22) DPI A-COOH | | | | | | | | | |
| | 0.44 | 0.17 | Apr. 16, 2008 | 614 | | | | | |
| IGF-1 A (H8 A9 N21) | 0.72 | 0.098 | Dec. 14, 2007 | | | | | | |
| B(H5D10Y16L17A22) (S═O) DPI A- | | | | | | | | | |
| COOH | | | | | | | | | |

* All C-terminals are amides unless specified otherwise.

### EXAMPLE 12

Dipeptide Half Life on IGF1 Dipeptide Extended (p-NH₂—F)^{A19}amide derivatives

The cleavage of an (pNH2-Phe) amide linked dipeptide AibPro from various

IGF-1 peptides was measured to determine the impact of the peptide sequence or heteroduplex on the dipeptide cleavage. Results for the tested peptides is shown in Table 12 and the data reveals that the IGF1-A chain alone represents a good model for the study of prodrug half life for IGF1 B:A (Y^{B16}L^{B17}) peptides.

TABLE 12

| Parent Peptide | Half Life (hr) |
|---|---|
| IGF1 A(Ala)^{6,11,20}(pNH₂-Phe)^{A19} | 2.2 |
| IGF1 A(Acm)^{6,11,20}(pNH₂-Phe)^{A19} | 1.8 |
| IGF1 B:A(S-S)^{A7,B7}(Acm)^{A6,11,20,B19}(pNH₂-Phe)^{A19} | 1.8 |
| IGF1 B:A(pNH₂-Phe)^{A19} | 1.6 |

Comparison of prodrug derivatives of the IGF A-chain relative to the disulfide bound A chain and B chain construct (IGF1 A:B(Y^{B16}L^{B17})) revealed the two compounds had similar half lives for the prodrug form. Note the AibAla derivative does not cleave and thus is not a prodrug, but serves to show the modification can inactivate the insulin analog IGF1 A:B(Y^{B16}L^{B17})(p-NH₂—F)^{A19}amide. Accordingly, the IGF1A chain alone was determined to be a good model for the study of pro-drug half life on IGF1 B:A (Y^{B16}L^{B17}) derivative peptides. Note the AibAla derivative does not cleave and thus is not a prodrug, but serves to show the modification can inactivate the insulin analog IGF1 A:B(Y^{B16}L^{B17})(p-NH₂—F)^{A19}amide. For simplicity, prodrug half lives were determined using only the IGF1 A chain in the absence of the B chain. The half lives of each propeptide was determined as described in Example 5. The data is presented in Table 13:

TABLE 13
Dipeptide half life on IGF1 dipeptide extended (p-NH₂-F)^{A19} amide
derivatives

| Dipeptide | | Half Life (hr) |
|---|---|---|
| AiB | Pro | 2.2 |
| AiBOH | Pro | 165.0 |
| AiB | dPro | 1.9 |
| AiBOH | Sar | 2.3 |
| dK (acetyl) | Sar | 16.3 |
| K | Sar | 21.8 |
| K (acetyl) | N-methyl Ala | 23.6 |
| dK (acetyl) | N-methyl Ala | 35.3 |

The data shows that by altering the substituents on the dipeptide prodrug element that the half life of prodrug can be varied from 2 hrs to >100 hrs.

Additional prodrug derivative peptides were prepared using an IGF1-A(pNH2-F)¹⁹ base peptide and altering the amino acid composition of the dipeptide prodrug element linked through the 4-amino phenylalanine at position A19. Dipeptide half lives were measured for different constructs both in PBS and in 20% plasma/PBS (i.e. in the presence of serum enzymes. The results are provided in Table 14. The results indicate that three of the four peptides tested were not impacted by serum enzymes.

TABLE 14
Dipeptide half life on IGF1-A(pNH2-F)¹⁹

| | | Half Life (hr) | |
|---|---|---|---|
| | | | 20% |
| | | PBS | Plasma/PBS |
| AiB | Pro | 2.2 | 2.1 |
| AiB | dPro | 2.1 | 2.2 |
| AiBOH | Sar | 2.3 | |
| dK | N-isobutyl Gly | 4.4 | 4.1 |
| dK | N-hexyl Gly | 10.6 | |
| dK (acetyl) | Sar | 17.2 | |
| K | Sar | 21.8 | 5.9 |
| K (acetyl) | N-methyl Ala | 23.6 | |
| dK (acetyl) | N-methyl Ala | 35.3 | |
| AiBOH | Pro | 165.0 | |
| K (acetyl) | Azetidine-2-carboxylic acid | Not cleavable | |
| dK (acetyl) | Azetidine-2-carboxylic acid | Not cleavable | |

### EXAMPLE 13

### Receptor Binding of IGF^{B16B17} Derivative Peptides Over Time

Prodrug formulations of IGF^{B16B17} Derivative Peptides were prepared and their degradation over time was measured using the insulin receptor binding assay of Example 3. Peptides used in the assay were prepared as follows:

### Dipeptide-IGF1A Analogs

If not specified, Boc-chemistry was applied in the synthesis of designed peptide analogs. Selected dipeptide H₂N-AA1-AA2-COOH was added to (pNH₂-Phe)¹⁹ on IGF1A (Ala)^{6,7,11,20}. The IGF-1 A chain C-terminal tripeptide Boc(Fmoc-pNH-Phe)-Ala-Ala was synthesized on MBHA resin. After removal of Fmoc by the treatment with 20% piperidine/DMF at room temperature for 30 minutes, Fmoc-AA2 was coupled to the p-amino benzyl side chain at A19 by using a threefold excess of amino acid, PyBop, DIEA and catalytic amount of pyridine. The Boc-synthesis of the remaining IGF-1 A chain (Ala)^{6,7,11,20} sequence was completed using an Applied Biosystems 430A Peptide Synthesizer, yielding IGF-1 A chain (Boc)⁰(Ala)^{6,7,11,20}(Fmoc-AA2-pNH-Phe)¹⁹-MBHA. After the Fmoc group was removed from the N-terminus of AA2, Boc-AA1 was then coupled to the amine using threefold excess of amino acid, DEPBT and DIEA. Removal of the two Boc groups remaining on the A chain by TFA was followed by HF cleavage, yielding IGF-1 A-chain (Ala)^{6,7,11,20}(H₂N-AA1-AA2-pNH-Phe)¹⁹amide. In the case of AA1 being d-lysine, acetylation on the ε-amine was performed prior to Boc removal. Dipeptide-IGF-1 A chain analogs were purified by semi-preparative RP-HPLC and characterized by analytical RP-HPLC and MALDI mass spectrometry.

### Dipeptide-IGF-1 (YL) Analogs

A selected dipeptide H₂N-AA1-AA2-COOH was added to (pNH₂-Phe)¹⁹ on IGF-1 A chain (Acm)^{6,11,20} as described immediately above except PAM resin was used for the synthesis of IGF-1 A chain to yield a C terminal acid upon HF-cleavage. IGF-1 B chain (Y^{B16}L^{B17})(Acm)¹⁹ was synthesized on MBHA resin to yield a C terminal amide. The free thiol on Cys^{B7} was modified by Npys through reaction with DTNP at a 1:1 molar ratio in 100% DMSO. Purified dipeptide-IGF-1 A chain and IGF-1 B chain (Y^{B16}L^{B17}) derivatives were assembled using the “1+2” two step chain combination strategy illustrated in Scheme 1. Intermediate and final purifications were performed on semi-preparative RP-HPLC and characterized by analytical RP-HPLC and MALDI mass spectrometry.

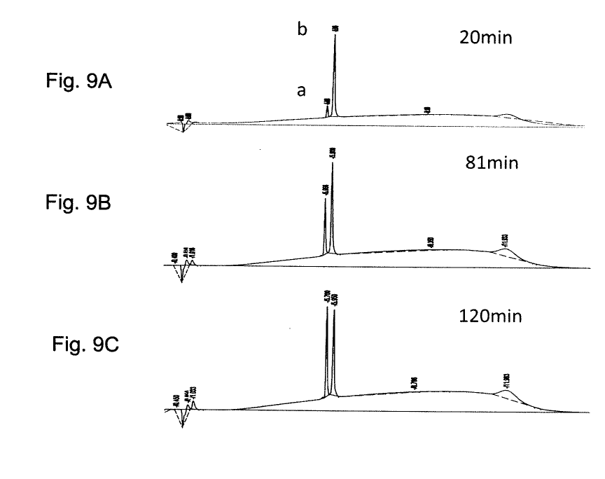

The IGF^{B16B17} derivative peptide prodrugs were incubated in PBS, pH 7.4 at 37° C. and at predetermined time intervals an aliquot was taken and further degradation was quenched with 0.1% TFA and the aliquot was subjected to analytical HPLC analysis. Peaks a and b, representing the prodrug and active forms of the IGF^{B16B17} derivative peptide were identified with LC-MS and quantified by integration of peak area an HPLC. FIGS. 9A-9C show the output of an HPLC analysis of the degradation of the IGF^{B16B17} derivativepeptide prodrug: IGF1A(Ala)^{6,7,11,20}(Aib-Pro-pNH-F)¹⁹. Aliquots were taken at 20 minutes (FIG. 9A), 81 minutes (FIG. 9B) and 120 minutes (FIG. 9C) after beginning the incubation of the prodrug in PBS. The data indicate the spontaneous, non-enzymatic conversion of IGF1A(Ala)^{6,7,11,20}(Aib-Pro-pNH—F)¹⁹ amide to IGF1A(Ala)^{6,7,11,20}(pNH₂—F)¹amide over time.

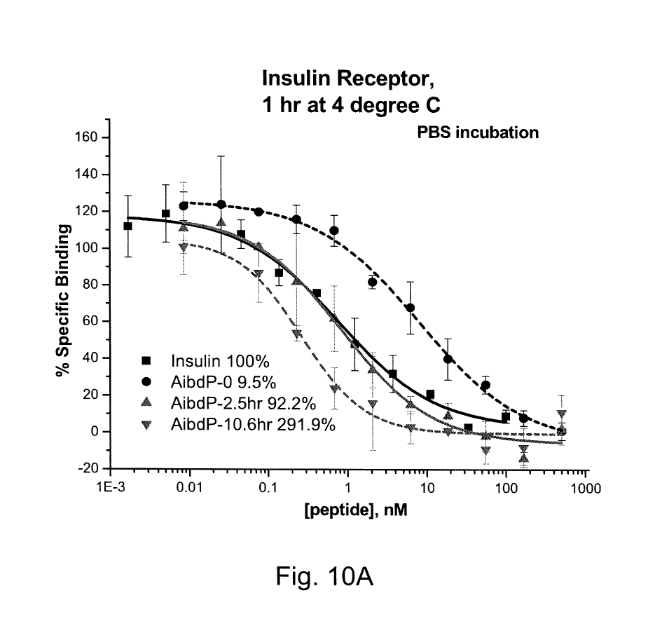

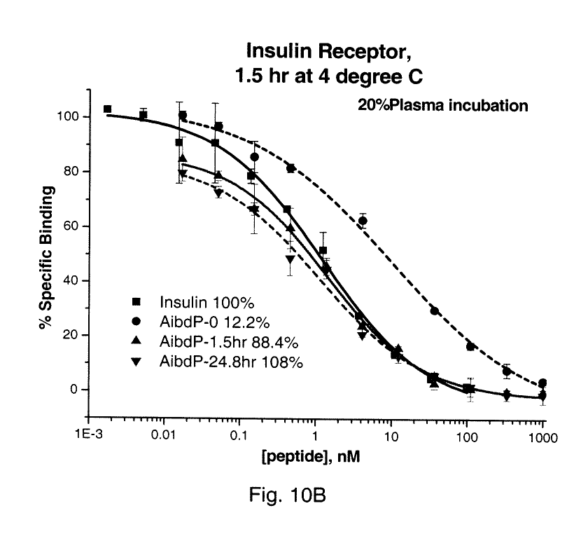

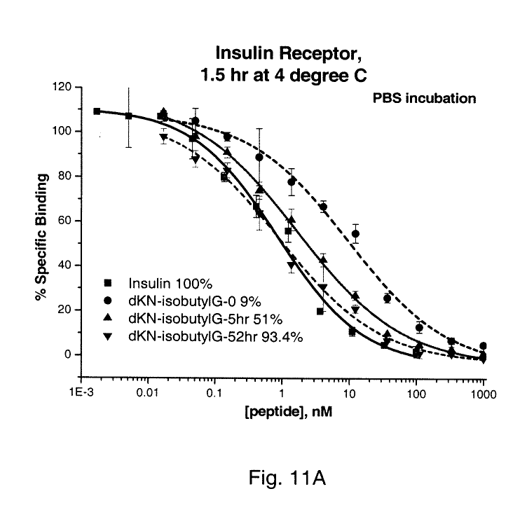

The degradation of the prodrug forms of IGF^{B16B17} derivative peptides to their active form was also measured based on the compounds ability to bind to the insulin receptor as measured using the in vitro assay of Example 3. FIGS. 10A & 10B are graphs depicting the in vitro activity of the prodrug Aib,dPro-IGF1YL (dipeptide linked throught the A19 4-aminoPhe). FIG. 10A is a graph comparing relative insulin receptor binding of native insulin (measured at 1 hour at 4° C.) and the A19 IGF prodrug analog (Aib,dPro-IGF1YL) over time (0 hours, 2.5 hours and 10.6 hours) incubated in PBS. FIG. 10B is a graph comparing relative insulin receptor binding of native insulin (measured at 1.5 hour at 4° C.) and the A19 IGF prodrug analog (Aib,dPro-IGF1YL) over time (0 hours, 1.5 hours and 24.8 hours) incubated in 20% plasma/PBS. As indicated by the data presented in the graph, increased activity is recovered form the A19 IGF prodrug analog sample as the prodrug form is converted to the active IGF1YL peptide. The activity of the IGF^{B16B17} derivative peptides was measured relative to insulin receptor binding, and since the underlying IGF^{B16B17} derivative peptides have more activity than native insulin, activity of greater than 100% relative to insulin is possible.

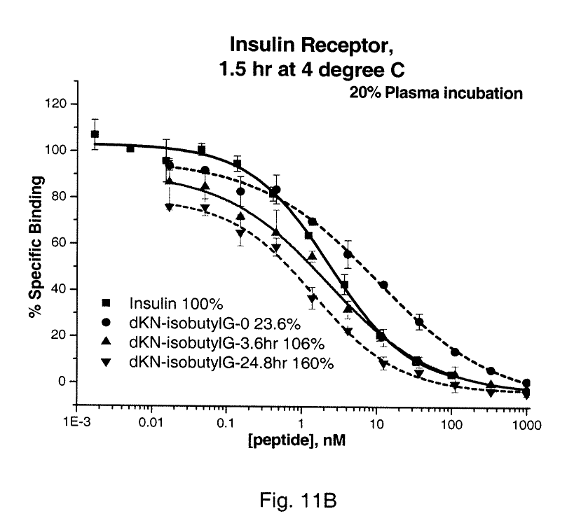

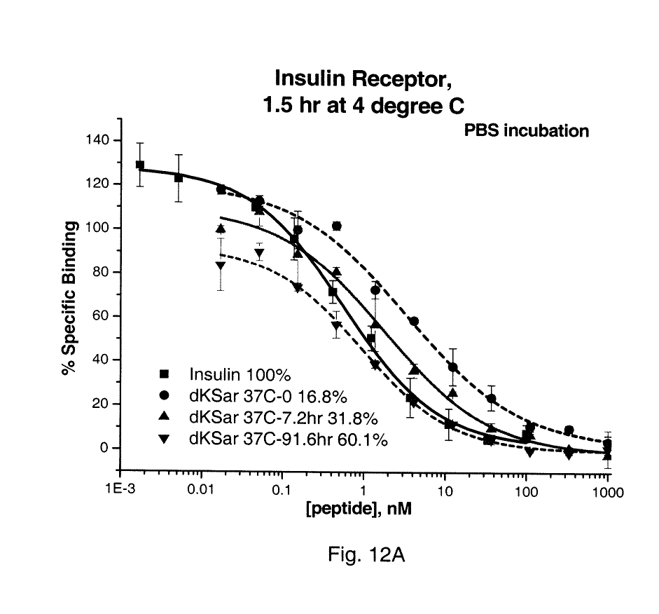

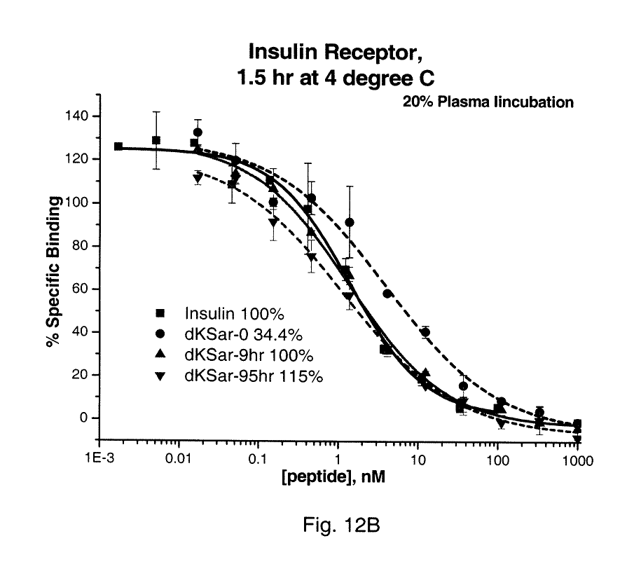

FIGS. 11A & 11B are graphs depicting the in vitro activity of the prodrug dK,(N-isobutylG)-IGF1YL (dipeptide linked throught the A19 4-aminoPhe). FIG. 11A is a graph comparing relative insulin receptor binding of native insulin (measured at 1 hour at 4° C.) and the A19 IGF prodrug analog (IGF1YL: dK,(N-isobutylG) over time (0 hours, 5 hours and 52 hours) incubated in PBS. FIG. 11B is a graph comparing relative insulin receptor binding of native insulin (measured at 1.5 hour at 4° C.) and the A19 IGF prodrug analog (IGF1YL: dK,(N-isobutylG) over time (0 hours, 3.6 hours and 24.8 hours) incubated in 20% plasma/PBS. As indicated by the data presented in the graph, increased activity is recovered form the A19 IGF prodrug analog sample as the prodrug form is converted to the active IGF1YL peptide. FIGS. 12A & 12B are graphs depicting the in vitro activity of the prodrug dK(e-acetyl),Sar)-IGF1YL (dipeptide linked throught the A19 4-aminoPhe). FIG. 12A is a graph comparing relative insulin receptor binding of native insulin (measured at 1 hour at 4° C.) and the A19 IGF prodrug analog (IGF1YL: dK(e-acetyl),Sar) over time (0 hours, 7.2 hours and 91.6 hours) incubated in PBS. FIG. 12B is a graph comparing relative insulin receptor binding of native insulin (measured at 1.5 hour at 4° C.) and the A19 IGF prodrug analog (IGF1YL: dK(e-acetyl),Sar) over time (0 hours, 9 hours and 95 hours) incubated in 20% plasma/PBS. As indicated by the data presented in the graph, increased activity is recovered form the A19 IGF prodrug analog sample as the prodrug form is converted to the active IGF1YL peptide.

1-54. (canceled)

55. An insulin analog comprising an A chain and a B chain wherein said A chain comprises a sequence GIVX₄ECCX₈X₉SCDLX₁₄X₁₅LEX₁₈X₁₉CX₂₁—R₁₃ (SEQ ID NO: 19), and said B chain comprises a sequence X₂₅LCGX₂₉X₃₀LVX₃₃X₃₄LYLVCGDX₄₂GFY (SEQ ID NO: 9) wherein
X₄ is glutamic acid or aspartic acid;
X₈ is histidine or phenylalanine;
X₉ and X₁₄ are independently selected from arginine, ornithine or alanine;
X₁₅ is arginine, alanine, ornathine or leucine;
X₁₈ is methionine, asparagine or threonine;
X₁₉ is tyrosine, 4-methoxy-phenylalanine or 4-amino phenylalanine;
X₂₁ is alanine, glycine or asparagine;
X₂₅ is selected from the group consisting of histidine and threonine;
X₂₉ is selected from the group consisting of alanine, glycine and serine;
X₃₀ is selected from the group consisting of histidine, aspartic acid, glutamic acid, homocysteic acid and cysteic acid;
X₃₃ is selected from the group consisting of aspartic acid and glutamic acid;
X₃₄ is selected from the group consisting of alanine and threonine;
X₄₂ is selected from the group consisting of alanine, ornithine and arginine; and
R₁₃ is COOH or CONH₂.

56. The insulin analog of claim 55 wherein the B chain comprises the Sequence R₂₂-X₂₅LCGX₂₉X₃₀LVX₃₃X₃₄LX₃₆LVCGDX₄₂GFX₄₅—R₄₇—R₄₈—R₄₉—R₁₄ (SEQ ID NO: 20), wherein
X₂₅ is histidine or threonine;
X₂₉ is selected from the group consisting of alanine, glycine and serine;
X₃₀ is selected from the group consisting of histidine, aspartic acid, glutamic acid, homocysteic acid and cysteic acid;
X₃₃ is selected from the group consisting of aspartic acid and glutamic acid;
X₃₄ is selected from the group consisting of alanine and threonine;
X₃₆ is tyrosine;
X₄₂ is selected from the group consisting of alanine, ornithine and arginine;
X₄₅ is tyrosine;
R₂₂ is selected from the group consisting of AYRPSE (SEQ ID NO: 14), PGPE (SEQ ID NO: 68), a tripeptide glycine-proline-glutamic acid, a dipeptide proline-glutamic acid, glutamic acid and an N-terminal amine;
R₄₇ is a phenylalanine-asparagine dipeptide, a phenylalanine-serine dipeptide or a tyrosine-threonine dipeptide;
R₄₈ is an aspartate-lysine dipeptide, an arginine-proline dipeptide, a proline-arginine dipeptide, a lysine-proline dipeptide, or a proline-lysine dipeptide;
R₄₉ is threonine or alanine; and
R₁₄ is COOH or CONH₂.

57. The insulin analog of claim 56 wherein
X₄ is aspartic acid;
X₈ is phenylalanine or histidine;
X₉ is arginine, ornathine or alanine,
X₂₁ is alanine, glycine or asparagine;
X₂₅ is histidine or threonine;
X₂₉ is alanine;
X₃₀ is glutamic acid or aspartic acid;
X₃₃ is aspartic acid;
X₃₄ is alanine;
R₂₂ is a glycine-proline-glutamic acid tripeptide;
R₄₇ is a phenylalanine-asparagine dipeptide;
R₄₈ is an aspartate-lysine dipeptide or a lysine-proline dipeptide;
R₄₉ is threonine;
R₁₃ is COOH; and
R₁₄ is CONH₂.

58. The insulin analog of claim 55, wherein the A chain comprises the sequence GIVDECCX₈X₉SCDLX₁₄X₁₅LEX₁₈YCX₂₁—R₁₃ (SEQ ID NO: 10), and the B chain comprises the sequence X₂₅LCGX₂₉X₃₀LVX₃₃X₃₄LYLVCGDX₄₂GFY—R₁₄ (SEQ ID NO: 9, wherein
X₈ is histidine or phenylalanine;
X₉ and X₁₄ are independently selected from arginine or alanine;
X₁₅ is arginine or leucine;
X₁₈ is methionine, asparagine or threonine;
X₂₁ is alanine, glycine or asparagine;
X₂₅ is selected from the group consisting of histidine and threonine;
X₂₉ is selected from the group consisting of alanine, glycine and serine;
X₃₀ is selected from the group consisting of histidine, aspartic acid, glutamic acid, homocysteic acid and cysteic acid;
X₃₃ is selected from the group consisting of aspartic acid and glutamic acid;
X₃₄ is selected from the group consisting of alanine and threonine; and
X₄₂ is selected from the group consisting of alanine and arginine; and
R₁₃ and R₁₄ are independently COOH or CONH₂.

59. The insulin analog of claim 55 further comprising a hydrophilic moiety linked to an amino acid of the insulin analog.

60. The insulin analog of claim 59 wherein the hydrophilic moiety is polyethylene glycol linked to the N-terminal amino acid of the B chain, or an amino acid side chain at position 28 or 29 of the B-chain.

61. The insulin analog of claim 55 wherein said polpeptide is acylated at one or more positions selected from A9, A14, A15, B22, B28 or B29.

62. A dimer or multimer comprising a insulin analog of claim 55.

63. A pharmaceutical composition comprising the insulin analog of claim 55 and a pharmaceutically acceptable carrier.

64. A method of treating diabetes, said method comprising administering an effective amount of a pharmaceutical composition of claim 63.

65. An insulin-like growth factor analog comprising an A chain and a B chain wherein said A chain comprises a sequence of Z-GIVX₄ECCX₈X₉SCDLX₁₄X₁₅LEX₁₈X₁₉CX₂₁—R₁₃ (SEQ ID NO: 19) or a sequence that differs from SEQ ID NO: 19 by 1 to 3 amino acid modifications selected from positions 5, 8, 9, 10, 14, 15, 17, 18 and 21 of SEQ ID NO: 19, and said B chain sequence comprises a sequence of J-R₂₂—X₂₅LCGX₂₉X₃₀LVX₃₃X₃₄LX₃₆LVCGDX₄₂GFX₄₅ (SEQ ID NO: 20) or a sequence that differs from SEQ ID NO: 20 by 1 to 3 amino acid modifications selected from positions 5, 6, 9, 10, 16, 18, 19 and 21 of SEQ ID NO: 20;
wherein Z and J are independently H or a dipeptide element comprising the general structure of U—O, wherein U is an amino acid or a hydroxyl acid and O is an N-alkylated amino acid;
X₄ is aspartic acid or glutamic acid;
X₈ is histidine or phenylalanine;
X₉ and X₁₄ are independently selected from arginine or alanine;
X₁₅ is arginine or leucine;
X₁₈ is methionine, asparagine or threonine;
X₁₉ is an amino acid of the general structure: 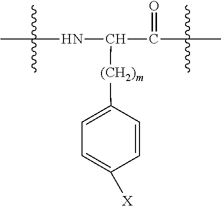
wherein X is selected from the group consisting of OH or NHR₁₀, wherein R₁₀ is a dipeptide element comprising the general structure U—O, wherein U is an amino acid or a hydroxyl acid and O is an N-alkylated amino acid;
X₂₁ is alanine, glycine or asparagine;
R₂₂ is selected from the group consisting of a covalent bond, AYRPSE (SEQ ID NO: 14), FGPE (SEQ ID NO: 68), a tripeptide glycine-proline-glutamic acid, a dipeptide proline-glutamic acid, and glutamic acid;
X₂₅ is selected from the group consisting of histidine and threonine;
X₂₉ is selected from the group consisting of alanine, glycine and serine;
X₃₀ is selected from the group consisting of histidine, aspartic acid, glutamic acid, homocysteic acid and cysteic acid;
X₃₃ is selected from the group consisting of aspartic acid and glutamic acid;
X₃₄ is selected from the group consisting of alanine and threonine;
X₃₆ is an amino acid of the general structure 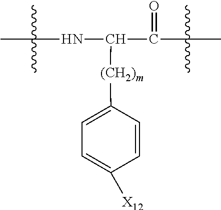
wherein X₁₂ is selected from the group consisting of OH and NHR₁₁, wherein R₁₁ is a dipeptide element comprising the general structure U—O;
X₄₂ is selected from the group consisting of alanine and arginine;
X₄₅ is an amino acid of the general structure 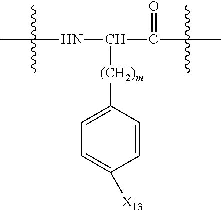
wherein X₁₃ is selected from the group consisting of OH and NHR₁₂, wherein R₁₂ is a dipeptide element comprising the general structure U—O;
m is an integer selected from 0-3; and
R₁₃ is COOH or CONH₂, with the proviso that one and only one of X, X₁₂, X₁₃, J and Z comprises U—O.

66. The insulin-like growth factor analog of claim 65 wherein U, O, or the amino acid of the insulin-like growth factor analog to which U—O is linked is a non-coded amino acid.

67. The insulin-like growth factor analog of claim 65 wherein m is 1; and
U—O comprises the dipeptide element of Formula I: 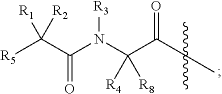
wherein
R_{1,} R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W)C₁-C₁₂ alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;
R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl or R₄ and R₃ together with the atoms to which they are attached form a 4, 5 or 6 member heterocyclic ring;
R₅ is NHR₆ or OH;
R₆ is H, C₁-C₈ alkyl or R₆ and R₂ together with the atoms to which they are attached form a 4, 5 or 6 member heterocyclic ring; and
R₇ is selected from the group consisting of H and OH;

68. The insulin-like growth factor analog of claim 67 wherein the A chain comprises the sequence Z-GIVDECCX₈X₉SCDLRRLEMX₁₉CX₂₁—R₁₃ (SEQ ID NO: 21) and a B chain having the sequence J-R₂₂—X₂₅LCGAX₃₀LVDALYLVCGDX₄₂GFYFN—R₄₈—R₄₉—R₁₄ (SEQ ID NO: 15), wherein
R₂₂ is AYRPSE (SEQ ID NO: 14) or a glycine-proline-glutamic acid tripeptide;
R₄₈ is an aspartate-lysine dipeptide, an arginine-proline dipeptide, a lysine-proline dipeptide, or a proline-lysine dipeptide;
R₄₉ is threonine;
R₁₃ is COOH; and
R₁₄ is CONH₂.

69. The insulin-like growth factor analog of claim 67 wherein said dipeptide element is pegylated with one or two polyethylene glycol chains wherein the combined molecular weight of the polyethylene glycol chains ranges from about 20,000 to about 80,000 Daltons.

70. The insulin-like growth factor analog of claim 67 wherein said dipeptide element is acylated with an acyl group comprising 16 to 30 carbon atoms.

71. The insulin-like growth factor analog of claim 66 wherein
Z and J are each H;
X₁₂ and X₁₃ are each OH;
X is NHR₁₀.

72. The insulin-like growth factor analog of claim 66 wherein
R₁ and R₂ are independently C₁-C₁₈ alkyl or aryl;
R₃ is C₁-C₁₈ alkyl or R₃ and R₄ together with the atoms to which they are attached form a 4-12 heterocyclic ring;
R₄ and R₈ are independently selected from the group consisting of hydrogen, C₁-C₁₈ alkyl and aryl; and
R₅ is an amine or a hydroxyl.

73. The insulin-like growth factor analog of any of claim 66, wherein
R₁ is selected from the group consisting of hydrogen, C₁-C₁₈ alkyl and aryl, or R₁ and R₂ are linked through —(CH₂)ₚ—, wherein p is 2-9;
R₃ is C₁-C₁₈ alkyl or R₃ and R₄ together with the atoms to which they are attached form a 4-6 heterocyclic ring;
R₄ and R₈ are independently selected from the group consisting of hydrogen, C₁-C₁₈ alkyl and aryl; and
R₅ is an amine or N-substituted amine.

74. The insulin-like growth factor analog of claim 66 wherein a side chain of one of the amino acids comprising the dipeptide element of Formula I further comprises a depot polymer.

75. The insulin-like growth factor analog of claim 74 wherein the depot polymer is polyethylene glycol.

76. A dimer or multimer comprising an insulin-like growth factor analog of claim 55.

77. An insulin-like growth factor analog of claim 55, wherein the dipeptide element amino acid corresponding to U is an amino acid in the D-stereochemical configuration.

78. An insulin-like growth factor analog of claim 55, wherein the carboxy terminus of the B chain is linked through a peptide linker to the N-terminus of said A chain to form a contiguous amino acid sequence.

79. A pharmaceutical composition comprising the insulin-like growth factor analog of claim 55, and a pharmaceutically acceptable carrier.

80. A method of treating diabetes, said method comprising administering an effective amount of a pharmaceutical composition of claim 79.


SEQUENCE LISTING (84 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 4 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 5 - protein, 21 aa, Homo sapiens
GIVDECCFRSCDLRRLEMYCA

SEQ ID NO: 6 - protein, 29 aa, Homo sapiens
GPETLCGAELVDALQFVCGDRGFYFNKPT

SEQ ID NO: 7 - protein, 21 aa, Homo sapiens
GIVEECCFRSCDLALLETYCA

SEQ ID NO: 8 - protein, 32 aa, Homo sapiens
AYRPSETLCGGELVDTLQFVCGDRGFYFSRPA

SEQ ID NO: 9 - protein, 21 aa, Artificial Sequence
  Analog of the insulin B chain
XLCGXXLVXXLYLVCGDXGFY

SEQ ID NO: 10 - protein, 21 aa, Artificial Sequence
  Analog of the insulin A chain
GIVDECCXXSCDLXXLEXYCX

SEQ ID NO: 11 - protein, 29 aa, Artificial sequence
  The “YL” analog of the IGF-2 B chain
GPETLCGAELVDALYLVCGDRGFYFNKPT

SEQ ID NO: 12 - protein, 32 aa, Artificial sequence
  The “YL” Analog of the IGF-2 B chain
AYRPSETLCGGELVDTLYLVCGDRGFYFSRPA

SEQ ID NO: 13 - protein, 21 aa, Artificial Sequence
  Analog of the insulin A chain
GIVDECCXXSCDLXXLEXXCX

SEQ ID NO: 14 - protein, 6 aa, Homo sapiens
AYRPSE

SEQ ID NO: 15 - protein, 23 aa, Artificial Sequence
  Analog of the insulin B chain
XLCGAXLVDALYLVCGDXGFYFN

SEQ ID NO: 16 - protein, 21 aa, Artificial Sequence
  Analog of the insulin A chain
GIVDECCXXSCDLRRLEMYCX

SEQ ID NO: 17 - protein, 26 aa, Artificial Sequence
  Analog of the insulin B chain
XLCGAXLVDALYLVCGDRGFYFNKPT

SEQ ID NO: 18 - protein, 21 aa, Artificial Sequence
  Analog of the insulin B chain
XLCGAXLVDALYLVCGDXGFY

SEQ ID NO: 19 - protein, 21 aa, Artificial Sequence
  Analog of the insulin A chain
GIVXECCXXSCDLXXLEXXCX

SEQ ID NO: 20 - protein, 21 aa, Artificial Sequence
  Analog of the insulin B chain
XLCGXXLVXXLXLVCGDXGFX

SEQ ID NO: 21 - protein, 21 aa, Artificial Sequence
  Analog of the insulin A chain
GIVDECCXXSCDLRRLEMXCX

SEQ ID NO: 22 - protein, 21 aa, Artificial Sequence
  Analog of the insulin A chain
GIVDECCFRSCDLRRLEMXCA

SEQ ID NO: 23 - protein, 26 aa, Artificial sequence
  Analog of the IGF B chain
TLCGAELVDALXLVCGDRGFXFNKPT

SEQ ID NO: 24 - protein, 21 aa, Artificial Sequence
  Analog of the insulin A chain
GIVDECCHASCDLRRLEMXCN

SEQ ID NO: 25 - protein, 26 aa, Artificial Sequence
  Analog of the IGF-1 B chain
HLCGAELVDALXLVCGDAGFXFNKPT

SEQ ID NO: 26 - protein, 29 aa, Artificial Sequence
  Analog of the IGF-1 B chain
GPETLCGAELVDALYLVCGDRGFYFNKPT

SEQ ID NO: 27 - protein, 7 aa, Artificial Sequence
  peptide linker
GGGPGKR

SEQ ID NO: 28 - protein, 8 aa, Artificial Sequence
  peptide linker
GSSSRRAP

SEQ ID NO: 29 - protein, 7 aa, Artificial Sequence
  peptide linker
GGGGGKR

SEQ ID NO: 30 - protein, 7 aa, Artificial Sequence
  peptide linker
RRGGGGG

SEQ ID NO: 31 - protein, 9 aa, Artificial Sequence
  peptide linker
GGAPGDVKR

SEQ ID NO: 32 - protein, 9 aa, Artificial Sequence
  peptide linker
RRAPGDVGG

SEQ ID NO: 33 - protein, 9 aa, Artificial Sequence
  peptide linker
GGYPGDVKR

SEQ ID NO: 34 - protein, 9 aa, Artificial Sequence
  peptide linker
RRYPGDVGG

SEQ ID NO: 35 - protein, 9 aa, Artificial Sequence
  peptide linker
GGHPGDVKR

SEQ ID NO: 36 - protein, 9 aa, Artificial Sequence
  peptide linker
RRHPGDVGG

SEQ ID NO: 37 - protein, 7 aa, Artificial Sequence
  peptide linker
RRGPGGG

SEQ ID NO: 38 - protein, 59 aa, Artificial Sequence
  single chain human insulin analog
HLCGAELVEALYLVCGDAGFYFDLPTQPLALEGSLQKRGIVDECCHASCDLRRLEMXCN

SEQ ID NO: 39 - protein, 59 aa, Artificial Sequence
  single chain human insulin analog
TLCGAELVDALYLVCGDRGFYFDLPTQPLALEGSLQKRGIVDECCFRSCDLRRLEMXCA

SEQ ID NO: 40 - protein, 7 aa, Artificial Sequence
  peptide linker
AGRGSGK

SEQ ID NO: 41 - protein, 7 aa, Artificial Sequence
  peptide linker
AGLGSGK

SEQ ID NO: 42 - protein, 7 aa, Artificial Sequence
  peptide linker
AGMGSGK

SEQ ID NO: 43 - protein, 7 aa, Artificial Sequence
  peptide linker
ASWGSGK

SEQ ID NO: 44 - protein, 7 aa, Artificial Sequence
  peptide linker
TGLGSGQ

SEQ ID NO: 45 - protein, 7 aa, Artificial Sequence
  peptide linker
TGLGRGK

SEQ ID NO: 46 - protein, 7 aa, Artificial Sequence
  peptide linker
TGLGSGK

SEQ ID NO: 47 - protein, 7 aa, Artificial Sequence
  peptide linker
HGLYSGK

SEQ ID NO: 48 - protein, 7 aa, Artificial Sequence
  peptide linker
KGLGSGQ

SEQ ID NO: 49 - protein, 7 aa, Artificial Sequence
  peptide linker
VGLMSGK

SEQ ID NO: 50 - protein, 7 aa, Artificial Sequence
  peptide linker
VGLSSGQ

SEQ ID NO: 51 - protein, 7 aa, Artificial Sequence
  peptide linker
VGLYSGK

SEQ ID NO: 52 - protein, 7 aa, Artificial Sequence
  peptide linker
VGLSSGK

SEQ ID NO: 53 - protein, 7 aa, Artificial Sequence
  peptide linker
VGMSSGK

SEQ ID NO: 54 - protein, 7 aa, Artificial Sequence
  peptide linker
VWSSSGK

SEQ ID NO: 55 - protein, 7 aa, Artificial Sequence
  peptide linker
VGSSSGK

SEQ ID NO: 56 - protein, 7 aa, Artificial Sequence
  peptide linker
VGMSSGK

SEQ ID NO: 57 - protein, 7 aa, Artificial Sequence
  peptide linker
TGLGSGR

SEQ ID NO: 58 - protein, 7 aa, Artificial Sequence
  peptide linker
TGLGKGQ

SEQ ID NO: 59 - protein, 7 aa, Artificial Sequence
  peptide linker
KGLSSGQ

SEQ ID NO: 60 - protein, 7 aa, Artificial Sequence
  peptide linker
VKLSSGQ

SEQ ID NO: 61 - protein, 7 aa, Artificial Sequence
  peptide linker
VKLSSGQ

SEQ ID NO: 62 - protein, 7 aa, Artificial Sequence
  peptide linker
TGLGKGQ

SEQ ID NO: 63 - protein, 7 aa, Artificial Sequence
  peptide linker
VGLSKGQ

SEQ ID NO: 64 - protein, 26 aa, Homo sapiens
TLCGAELVDALYLVCGDRGFYFNKPT

SEQ ID NO: 65 - protein, 21 aa, Artificial Sequence
  Analog of the insulin B chain
XLCGXELVDXLYLVCGDXGFY

SEQ ID NO: 66 - protein, 94 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCNPLKPAKSA

SEQ ID NO: 67 - protein, 21 aa, Artificial Sequence
  Analog of the insulin B chain
XLCGXXLVXXLYLVCGXXGFX

SEQ ID NO: 68 - protein, 4 aa, Artificial Sequence
  peptide linker
FGPE

SEQ ID NO: 69 - protein, 21 aa, Artificial Sequence
  Analog of the insulin A chain
GIVDECCXXSCDLRRLEMXCX

SEQ ID NO: 70 - protein, 21 aa, Artificial Sequence
  Analog of the insulin A chain
GIVDECCFRSCDLRRLEMXCA

SEQ ID NO: 71 - protein, 30 aa, Homo sapiens
FVNQTLCGAELVDALYLVCGDRGFYFNKPT

SEQ ID NO: 72 - protein, 30 aa, Homo sapiens; identical to SEQ ID NO: 71

SEQ ID NO: 73 - protein, 6 aa, Artificial Sequence
  synthetic peptide for testing cleavage of dipeptide prodrug element
HSRGTF

SEQ ID NO: 74 - protein, 8 aa, Artificial Sequence
  synthetic peptide for testing cleavage of dipeptide prodrug element
KXHSTGTF

SEQ ID NO: 75 - protein, 6 aa, Artificial Sequence
  glucagon analogue
XXXGXX

SEQ ID NO: 76 - protein, 8 aa, Artificial Sequence
  glucagon analogue
XXXXXGXX

SEQ ID NO: 77 - protein, 6 aa, Artificial Sequence
  Model hexapeptide for dipeptide cleavage experiments
HSRGTF

SEQ ID NO: 78 - protein, 8 aa, Artificial Sequence
  glucagon analogue
HXHSRGTF

SEQ ID NO: 79 - protein, 12 aa, Homo sapiens
GYGSSSRRAPQT

SEQ ID NO: 80 - protein, 8 aa, Homo sapiens
SRVSRRSR

SEQ ID NO: 81 - protein, 8 aa, Homo sapiens
GSSSRRAP

SEQ ID NO: 82 - protein, 21 aa, Artificial Sequence
  Analog of the insulin A chain
GIVXXCCXXXCXLXXLEXXCX

SEQ ID NO: 83 - protein, 48 aa, Artificial Sequence
  IGF1 B and A chain analogs
CGPEHLCGAELVDALYLVCGDXGFYFNGIVDECCFXSCDLXXLENYCN

SEQ ID NO: 84 - protein, 32 aa, Homo sapiens
FVNQTLCGAELVDALYLVCGDRGFYFNKPTEE